(19) United States
(12) Patent Application Publication
(10) Pub. No.: US 2024/0182485 A1
(43) Pub. Date: Jun. 6, 2024

(54) PROCESS FOR PREPARING SHP2 INHIBITORS

(71) Applicants: Genzyme Corporation, Cambridge, MA (US); Revolution Medicines, Inc., Redwood City, CA (US)

(72) Inventors: Mickaël ARNOLD, Cambridge, MA (US); Steven G. BALLMER, Redwood City, CA (US); Céline GARNIER, Cambridge, MA (US); Jean-Pierre LESZCZAK, Cambridge, MA (US); Shaoling LI, Redwood City, CA (US)

(21) Appl. No.: 18/210,476

(22) Filed: Jun. 15, 2023

Related U.S. Application Data
(63) Continuation of application No. PCT/US2021/064040, filed on Dec. 17, 2021.
(60) Provisional application No. 63/127,957, filed on Dec. 18, 2020.
(60) Provisional application No. 63/215,820, filed on Jun. 28, 2021.

(51) Int. Cl. C07D 491/107

(52) U.S. Cl. CPC C07D 491/107

(57) ABSTRACT

Provided herein are methods for preparing an SHP2 inhibitor and intermediates useful therein.

### CROSS-REFERENCE TO RELATED APPLICATIONS

This application claims priority to U.S. Provisional Application No. 63/127,957, filed on Dec. 18, 2021, and U.S. Provisional Application No. 63/215,820, filed on Jun. 28, 2021, the disclosures of each of which are hereby incorporated by reference in their entireties.

### FIELD OF THE DISCLOSURE

The present disclosure relates generally to methods for preparing inhibitors of SHP2 and intermediates useful therein.

### BACKGROUND OF THE DISCLOSURE

SH2 domain-containing protein tyrosine phosphatase-2 (SHP2) is a non-receptor protein tyrosine phosphatase encoded by the PTPN11 gene that contributes to multiple cellular functions including proliferation, differentiation, cell cycle maintenance, and migration. SHP2 is involved in signaling through the Ras-mitogen-activated protein kinase, the JAK-STAT, or the phosphoinositol 3-kinase-AKT pathways.

SHP2 has two N-terminal Src homology 2 domains (N—SH2 and C—SH2), a catalytic domain (PTP), and a C-terminal tail. The two SH2 domains control the subcellular localization and functional regulation of SHP2. The protein exists in an inactive, self-inhibited conformation stabilized by a binding network involving residues from both the N—SH2 and PTP domains. Certain molecules, such as cytokines or growth factors, stimulate SHP2 and lead to exposure of the catalytic site, resulting in enzymatic activation of SHP2.

Mutations in the PTPN11 gene and subsequently in SHP2 have been identified in several human diseases, such as Noonan Syndrome, Leopard Syndrome, juvenile myelomonocytic leukemias, neuroblastoma, melanoma, acute myeloid leukemia, breast cancers, lung cancers, and colon cancer. As such, SHP2 is an attractive target for the development of novel therapies for the treatment of these diseases.

U.S. Pat. No. 10,590,090 discloses the compound {6-[(2-amino-3-chloropyridin-4-yl)sulfanyl]-3-[3S,4S)-4-amino-3-methyl-2-oxa-8-azaspiro[4.5]decan-8-yl]-5-methylpyrazin-2-yl}methanol (referred to in the present disclosure as the “compound of Formula (7)” or “Compound (7)”) as an SHP2 inhibitor. The numbering of the two asymmetric carbon atoms in (S) absolute configuration (C₃ and C₄) is shown on the spiro-cycle below.

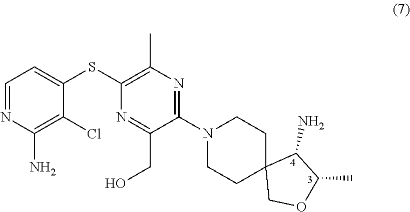 [SMILES: Cc1nc(N2CCC3(CC2)CO[C@@H](C)[C@H]3N)c(CO)nc1Sc1ccnc(N)c1Cl]

The synthesis of Compound (7) disclosed therein involves ten steps and requires chromatographic purification of diastereomers. Accordingly, a more efficient and selective synthesis of Compound (7) is desirable.

Accordingly, in one aspect, provided herein are improved methods for preparing Compound (7), or a salt thereof, and intermediates useful therein. Unlike the process disclosed in U.S. Pat. No. 10,590,090, the presently disclosed methods of preparing Compound (7), and intermediates thereof, do not require chromatographic purification of diastereomers and afford the desired product through a more efficient process.

### SUMMARY OF THE DISCLOSURE

Described herein, in certain embodiments, are methods for preparing Compound (7), or a salt thereof, and intermediates useful therein.

Provided herein is a method of preparing a compound of Formula (6):

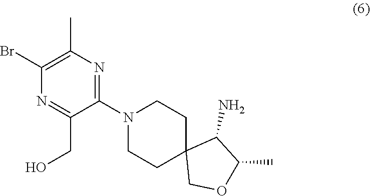 [SMILES: Cc1nc(N2CCC3(CC2)CO[C@@H](C)[C@H]3N)c(CO)nc1Br]

or a salt thereof, comprising:
reacting a compound of Formula (5):

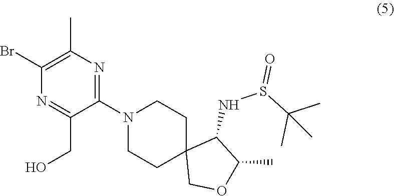 [SMILES: Cc1nc(N2CCC3(CC2)CO[C@@H](C)[C@H]3NS(=O)C(C)(C)C)c(CO)nc1Br]

with an acid to form the compound of Formula (6) or a salt thereof. In some embodiments, the acid is HCl, HBr, methanesulfonic acid, or acetic acid. In some embodiments, the compound of Formula (5) is the compound of Formula (5a):

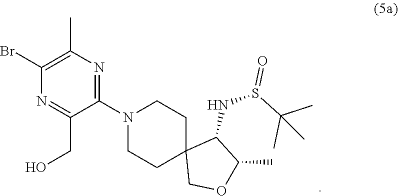 [SMILES: Cc1nc(N2CCC3(CC2)CO[C@@H](C)[C@H]3N[S@](=O)C(C)(C)C)c(CO)nc1Br]

In some embodiments, the compound of Formula (5) is prepared by reacting a compound of Formula (2):

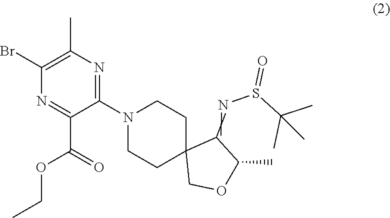 [SMILES: CCOC(=O)c1nc(Br)c(C)nc1N1CCC2(CC1)CO[C@@H](C)C2=NS(=O)C(C)(C)C]

with a reducing agent to form the compound of Formula (5). In some embodiments, the reducing agent is an organoaluminum hydride, an organoborane hydride, or a borohydride reagent. In some embodiments, the reducing agent is diisobutylaluminum hydride (DIBAL-H), LiBHEt₃, L-selectride, N-selectride, K-selectride, sodium borohydride, lithium borohydride, or potassium borohydride. In some embodiments, the compound of Formula (2) is the compound of Formula (2a):

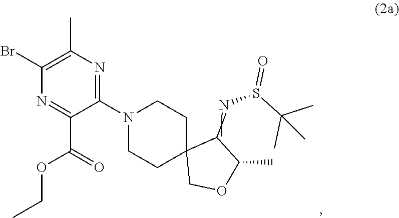 [SMILES: CCOC(=O)c1nc(Br)c(C)nc1N1CCC2(CC1)CO[C@@H](C)C2=N[S@](=O)C(C)(C)C]

and the compound of Formula (5) is the compound of Formula (5a):

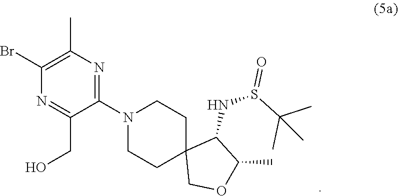 [SMILES: Cc1nc(N2CCC3(CC2)CO[C@@H](C)[C@H]3N[S@](=O)C(C)(C)C)c(CO)nc1Br]

In some embodiments, the compound of Formula (2) is prepared by reacting the compound of Formula (1):

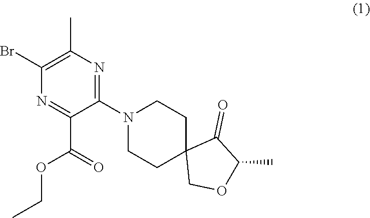 [SMILES: CCOC(=O)c1nc(Br)c(C)nc1N1CCC2(CC1)CO[C@@H](C)C2=O]

with the compound of Formula (A1):

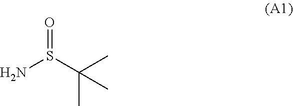

and a titanium alkoxide reagent to form the compound of Formula (2). In some embodiments, the titanium alkoxide reagent is Ti(OCH₂CH₃)₄.

In some embodiments, the compound of Formula (1) is prepared by reacting a compound of Formula (II):

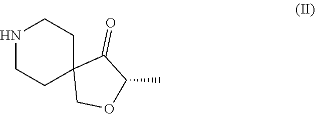

or a salt thereof,
with the compound of Formula (IV):

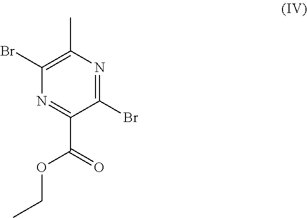

to form the compound of Formula (1). In some embodiments, the reaction of the compound of Formula (II), or a salt thereof, with the compound of Formula (IV) further comprises a base. In some embodiments, the base is K₂CO₃, Na₂CO₃, or NaHCO₃.

In some embodiments, the compound of Formula (II), or a salt thereof, is prepared by reacting a compound of Formula (I):

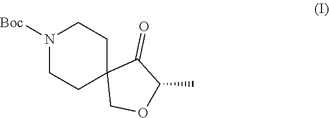 [SMILES: C[C@@H]1OCC2(CCN(C(=O)OC(C)(C)C)CC2)C1=O]

with an acid to form the compound of Formula (II) or a salt thereof. In some embodiments, the acid used for the reaction of the compound of Formula (I) is HCl or HBr.

In some embodiments, the compound of Formula (IV) is prepared by reacting a compound of Formula (III):

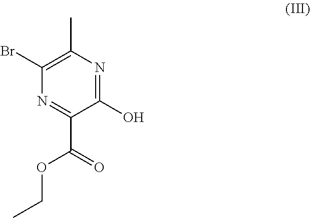

with POBr₃ to form the compound of Formula (IV).

Also provided herein is a method of preparing a compound of Formula (7):

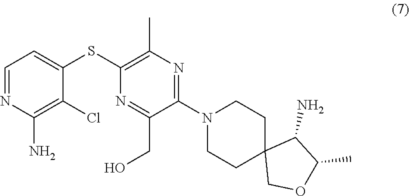 [SMILES: Cc1nc(N2CCC3(CC2)CO[C@@H](C)[C@H]3N)c(CO)nc1Sc1ccnc(N)c1Cl]

or a salt thereof, comprising reacting the compound of Formula (6), or a salt thereof, prepared according to any one of claims 1-15 with a compound of Formula (VI):

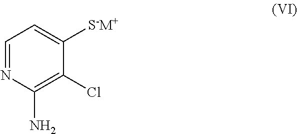

wherein M⁺ is Li⁺, Na⁺, or K⁺,
to form the compound of Formula (7) or a salt thereof. In some embodiments, the reaction of the compound of Formula (6), or a salt thereof, with the compound of Formula (VI) further comprises a copper salt.

In some embodiments, the compound of Formula (VI) is prepared by reacting a compound of Formula (V):

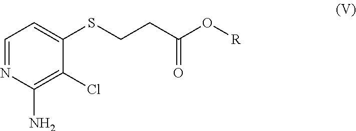

wherein R is C1-C12 alkyl,
with a base to form the compound of Formula (VI). In some embodiments, the compound of Formula (V) is the compound of Formula (Va):

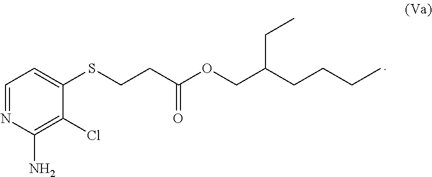 [SMILES: CCCCC(CC)COC(=O)CCSc1ccnc(N)c1Cl]

In some embodiments, the base used for the reaction of the compound of Formula (V) is NaOH, KOH, LiGH, KOCH₃, NaOCH₃, LiOCH₃, KOCH₂CH₃, NaOCH₂CH₃, LiOCH₂CH₃, NaO(tert-butyl), KO(tert-butyl), or LiO(tert-butyl).

Also provided herein is a method of preparing a compound of Formula (7), or a salt thereof, comprising the following steps:

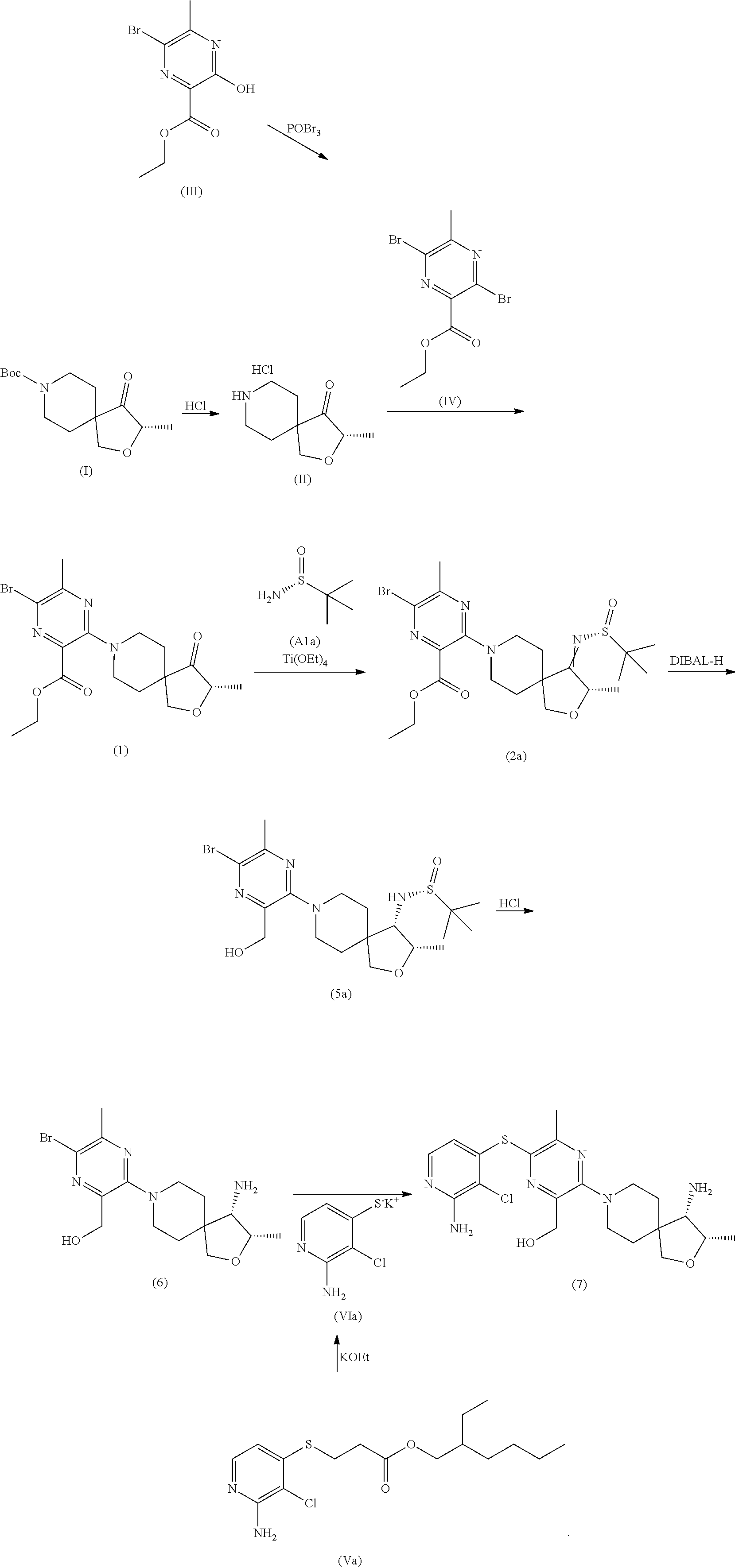

In a further aspect, provided herein is a method of preparing a compound of Formula (4):

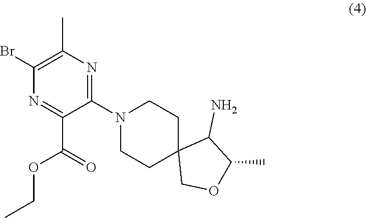 [SMILES: CCOC(=O)c1nc(Br)c(C)nc1N1CCC2(CC1)CO[C@@H](C)C2N]

or a salt thereof, comprising:
reacting a compound of Formula (3):

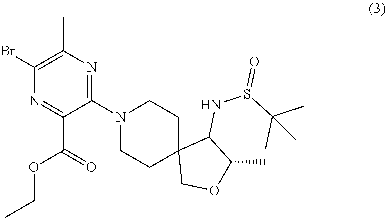 [SMILES: CCOC(=O)c1nc(Br)c(C)nc1N1CCC2(CC1)CO[C@@H](C)C2NS(=O)C(C)(C)C]

with an acid to form the compound of Formula (4) or a salt thereof. In some embodiments, the compound of Formula (3) is prepared by reacting a compound of Formula (2):

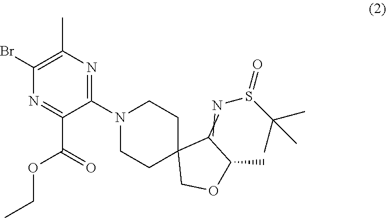 [SMILES: CCOC(=O)c1nc(Br)c(C)nc1N1CCC2(CC1)CO[C@@H](C)C2=NS(=O)C(C)(C)C]

with a reducing agent to form the compound of Formula (3).

Also provided herein is a compound of Formula (1):

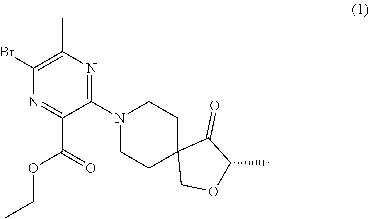 [SMILES: CCOC(=O)c1nc(Br)c(C)nc1N1CCC2(CC1)CO[C@@H](C)C2=O]

Also provided herein is a compound of Formula (2):

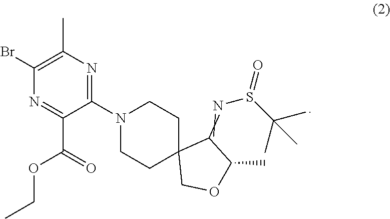 [SMILES: CCOC(=O)c1nc(Br)c(C)nc1N1CCC2(CC1)CO[C@@H](C)C2=NS(=O)C(C)(C)C]

Also provided herein is a compound of Formula (3):

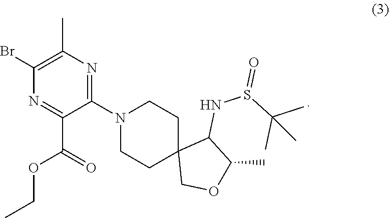 [SMILES: CCOC(=O)c1nc(Br)c(C)nc1N1CCC2(CC1)CO[C@@H](C)C2NS(=O)C(C)(C)C]

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the detailed descriptions are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosure.

As used herein, ranges and amounts can be expressed as “about” a particular value or range. About also includes the exact amount. Hence “about 0° C.” means “about 0° C.” and also “0° C.” Generally, the term “about” includes an amount that would be expected to be within experimental error, such as for example, within 15%, 10%, or 5%.

As used herein, the term “salt” refers to an acid or base salt of a compound disclosed herein. In some instances, the salt is a “pharmaceutically acceptable salt”, which is understood to be non-toxic. Non-limiting examples of salts include mineral acid (hydrochloric acid, hydrobromic acid, phosphoric acid, and the like) salts, organic acid (acetic acid, propionic acid, glutamic acid, citric acid, methanesulfonic acid, p-toluenesulfonic acid, and the like) salts, and quaternary ammonium (methyl iodide, ethyl iodide, and the like) salts.

Acid addition salts are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Non-limiting examples of inorganic salts include ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, non-limiting examples of which include ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁₀ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, 3-methylpentyl, 2-ethylhexyl, 3-ethylhexyl, and 4-ethylhexyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃)CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Halogen” or “halo” includes fluoro, chloro, bromo, and iodo.

“Therapeutically effective amount” of a compound or a composition refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

As used herein, the terms “subject(s)” and “patient(s)” mean any mammal. Examples include, but are not limited to, mice, rats, hamsters, guinea pigs, pigs, rabbits, cats, dogs, goats, sheep, cows, and humans. In some embodiments, the mammal is a human.

### Synthesis of Compounds

Provided herein are methods of preparing Compound (7) or a salt thereof. Also provided herein are intermediate compounds useful for the preparation of Compound (7), or a salt thereof, as well as the synthesis of such intermediates. An overview of a synthesis of Compound (7), or a salt thereof, of the present disclosure is shown in Scheme 1.

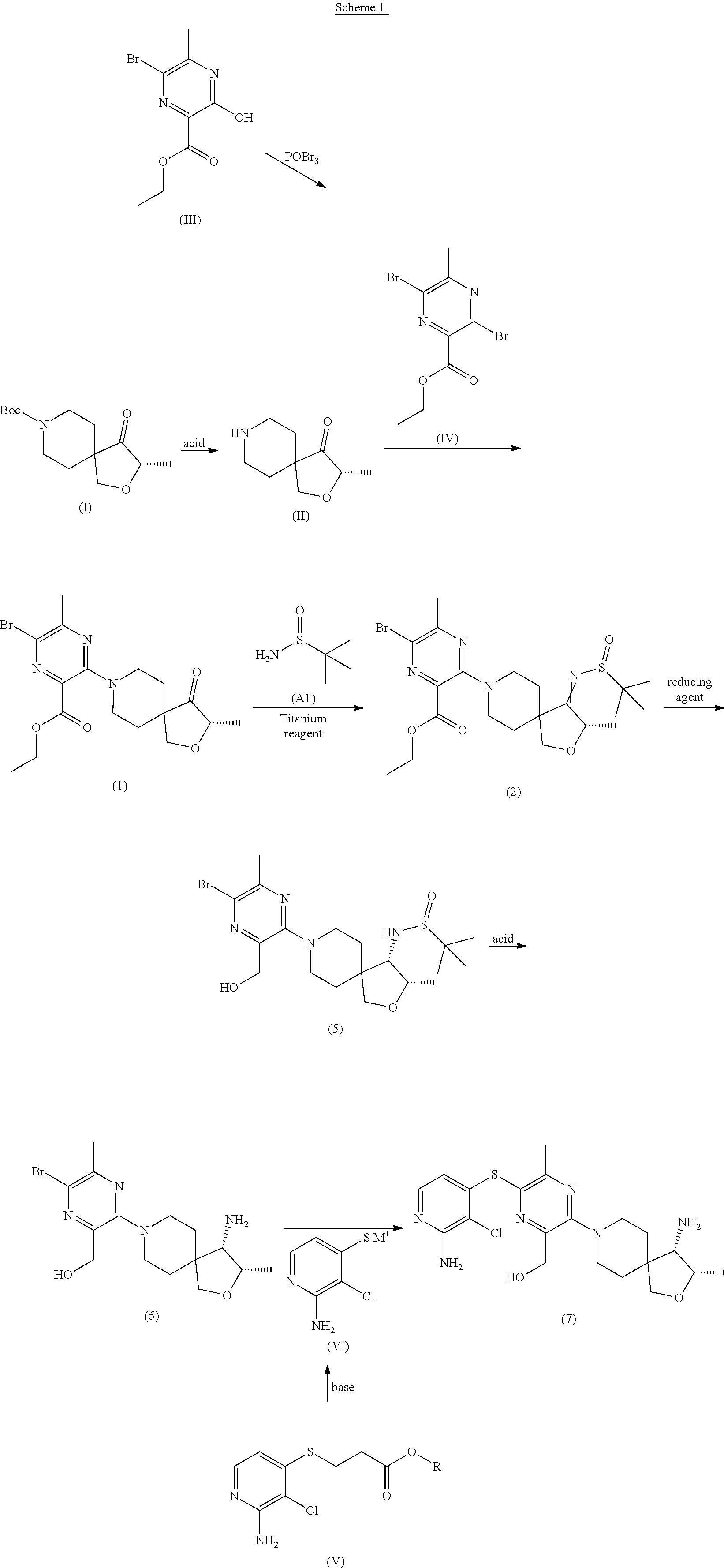

wherein M⁺ is Li⁺, Na⁺, or K⁺; R is C₁-C₁₂ alkyl; and [symbol] represents a double bond having either E or Z configuration. Throughout this description, a compound represented with [symbol] in its chemical structure indicates that the compound has a double bond in either E or Z configuration. In some embodiments, [symbol] is a double bond having E configuration. In other embodiments, [symbol] is a double bond having Z configuration.

In one aspect, provided herein are methods of preparing a compound of Formula (6) or a salt thereof. In some embodiments, provided herein is a method of preparing a compound of Formula (6), or a salt thereof, comprising reacting a compound of Formula (2) with a reducing agent to form a compound of Formula (5). In some embodiments, provided herein is a method of preparing a compound of Formula (6), or a salt thereof, comprising reacting a compound of Formula (1) with a compound of Formula (A1) and a titanium alkoxide reagent to form a compound of Formula (2). In some embodiments, provided herein is a method of preparing a compound of Formula (6), or a salt thereof, comprising reacting a compound of Formula (II), or a salt thereof, with a compound of Formula (IV) to form a compound of Formula (1). In some embodiments, provided herein is a method of preparing a compound of Formula (6), or a salt thereof, comprising reacting a compound of Formula (I) with an acid to form a compound of Formula (II) or a salt thereof. In some embodiments, provided herein is a method of preparing a compound of Formula (6), or a salt thereof, comprising reacting a compound of Formula (III) with POBr₃ to form a compound of Formula (IV).

### Compound of Formula (6)

In one aspect, provided herein is a method of preparing a compound of Formula (6):

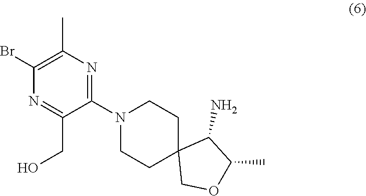 [SMILES: Cc1nc(N2CCC3(CC2)CO[C@@H](C)[C@H]3N)c(CO)nc1Br]

or a salt thereof, comprising:
reacting a compound of Formula (5):

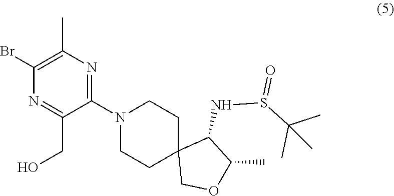 [SMILES: Cc1nc(N2CCC3(CC2)CO[C@@H](C)[C@H]3NS(=O)C(C)(C)C)c(CO)nc1Br]

with an acid to form the compound of Formula (6) or a salt thereof.

In some embodiments, the acid is HCl, HBr, methanesulfonic acid, trifluoroacetic acid, or acetic acid. In some embodiments, the acid is HCl. In some embodiments, HCl is generated in situ by reaction of acetyl chloride, trimethylsilyl chloride, or AlCl₃ with an alcohol, such as methanol or ethanol.

In some embodiments, the reaction is carried out using an alcohol as solvent. In some embodiments, the alcohol is ethanol, methanol, or isopropanol. In some embodiments, the alcohol is ethanol. In some embodiments, the alcohol is methanol. In some embodiments, the reaction is carried out using an ether, such as 1,4-dioxane, tetrahydrofuran, 2-methyltetrahydrofuran, or diethyl ether, as solvent. In some embodiments, the ether is tetrahydrofuran. In some embodiments, the reaction is carried out using a mixture of an ether and an alcohol, such as methanol or ethanol, as solvent. In some embodiments, the ether is 1,4-dioxane, tetrahydrofuran, 2-methyltetrahydrofuran, or diethyl ether. In some embodiments, the reaction is carried out using water as solvent. In some embodiments, the reaction is carried out using a mixture of water and an alcohol, such as methanol or ethanol. In some embodiments, the reaction is carried out using a biphasic solvent system. In some embodiments, the biphasic solvent system is a mixture of water and 2-methyltetrahydrofuran. In some embodiments, the reaction is carried out using an acidic biphasic solvent system, such as HCl in a mixture of water and 2-methyltetrahydrofuran. In some embodiments, the reaction is carried out using acidic ethyl acetate as solvent, such as HCl in ethyl acetate. In some embodiments, the reaction is carried out using acidic dioxane as solvent, such as HCl in dioxane.

In some embodiments, the reaction is carried out at a temperature of about 0-25° C. In some embodiments, the reaction temperature is about 22° C. In some embodiments, the reaction is carried out at a temperature of about 0-100° C., such as about 35-90° C.

In some embodiments, the compound of Formula (5) is the compound of Formula (5a):

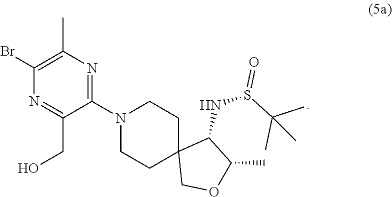 [SMILES: Cc1nc(N2CCC3(CC2)CO[C@@H](C)[C@H]3N[S@](=O)C(C)(C)C)c(CO)nc1Br]

### Compound of Formula (5)

In a further aspect, provided herein is a method of preparing a compound of Formula (5):

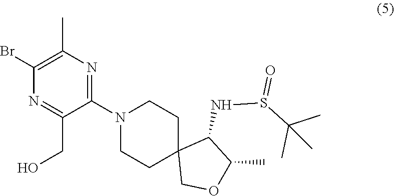 [SMILES: Cc1nc(N2CCC3(CC2)CO[C@@H](C)[C@H]3NS(=O)C(C)(C)C)c(CO)nc1Br]

comprising reacting a compound of Formula (2):

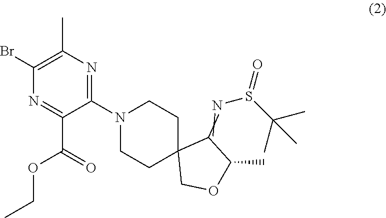 [SMILES: CCOC(=O)c1nc(Br)c(C)nc1N1CCC2(CC1)CO[C@@H](C)C2=NS(=O)C(C)(C)C]

with a reducing agent to form the compound of Formula (5).

In some embodiments, the reducing agent is an organoaluminum hydride, an organoborane hydride, or a borohydride reagent. In some embodiments, the reducing agent is diisobutylaluminum hydride (DIBAL-H), LiBHEt₃, L-selectride, N-selectride, K-selectride, sodium borohydride, lithium borohydride, or potassium borohydride. In some embodiments, the reducing agent is diisobutylaluminum hydride (DIBAL-H). In some embodiments, DIBAL-H is used as a neat liquid. In some embodiments, DIBAL-H is used as an organic solution of tetrahydrofuran, toluene, cyclohexane, heptane or dichloromethane.

In some embodiments, the reaction of the compound of Formula (2) with the reducing agent is carried out using an aprotic solvent. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is 2-methyltetrahydrofuran.

In some embodiments, the reaction of the compound of Formula (2) with the reducing agent is carried out at a temperature of about −15 to −25° C. In some embodiments, the reaction temperature is about −20° C. In some embodiments, the reaction is carried out at a temperature of about −60 to 25° C. In some embodiments, the reaction temperature is about −35° C. In some embodiments, the reaction temperature is about −10° C. In some embodiments, the reaction temperature is about −35 to −10° C. In some embodiments, the reaction of the compound of Formula (2) with the reducing agent is carried out at a temperature of about −40 to 20° C. In some embodiments, the reaction of the compound of Formula (2) with the reducing agent is carried out at a temperature of about −30 to 30° C. In some embodiments, the reaction temperature is about −40° C. In some embodiments, the reaction temperature is about 0° C. In some embodiments, the reaction temperature is about 10° C. In some embodiments, the reaction temperature is about 20° C.

In some embodiments, the compound of Formula (2) is the compound of Formula (2a):

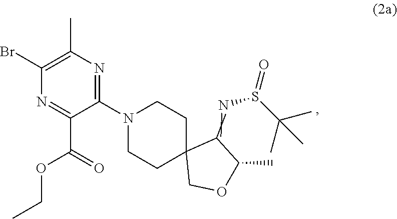 [SMILES: CCOC(=O)c1nc(Br)c(C)nc1N1CCC2(CC1)CO[C@@H](C)C2=N[S@](=O)C(C)(C)C]

and the compound of Formula (5) is the compound of Formula (5a):

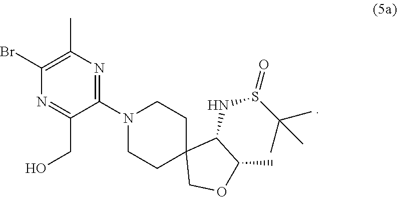 [SMILES: Cc1nc(N2CCC3(CC2)CO[C@@H](C)[C@H]3N[S@](=O)C(C)(C)C)c(CO)nc1Br]

### Compound of Formula (2)

Also provided herein is a compound of Formula (2):

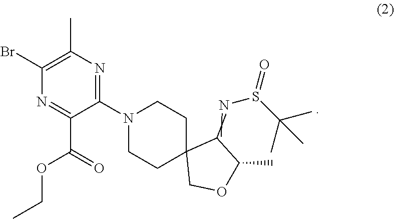 [SMILES: CCOC(=O)c1nc(Br)c(C)nc1N1CCC2(CC1)CO[C@@H](C)C2=NS(=O)C(C)(C)C]

Although the compound of Formula (2) is shown in the present disclosure as an ethyl ester, other alkyl esters such as methyl, propyl (e.g., n-propyl or isopropyl), or butyl (e.g., n-butyl or tert-butyl) may alternatively be used. In some variations wherein an alternate alkyl ester (such as methyl, propyl, or butyl) of the compound of Formula (2) is used, it follows that the compound of Formula (1) will similarly be provided as the alternate alkyl ester (methyl, propyl, or butyl).

In some embodiments, the compound of Formula (2) has the structure of Formula (2a):

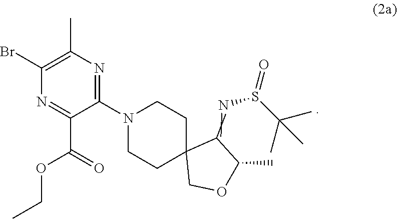 [SMILES: CCOC(=O)c1nc(Br)c(C)nc1N1CCC2(CC1)CO[C@@H](C)C2=N[S@](=O)C(C)(C)C]

In other embodiments, the compound of Formula (2) has the structure of Formula (2b):

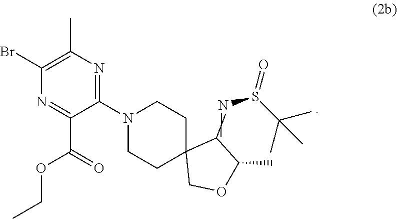 [SMILES: CCOC(=O)c1nc(Br)c(C)nc1N1CCC2(CC1)CO[C@@H](C)C2=N[S@@](=O)C(C)(C)C]

In the structures of Formula (2), Formula (2a), and Formula (2b), [symbol] represents a double bond having either E configuration or Z configuration. In some embodiments, [symbol] is a double bond having E configuration. In other embodiments, [symbol] is a double bond having Z configuration.

In a further aspect, provided herein is a method of preparing a compound of Formula (2):

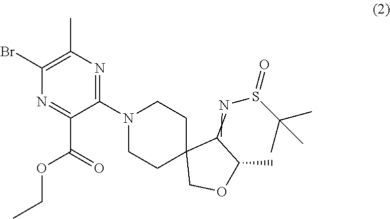 [SMILES: CCOC(=O)c1nc(Br)c(C)nc1N1CCC2(CC1)CO[C@@H](C)C2=NS(=O)C(C)(C)C]

comprising reacting the compound of Formula (1):

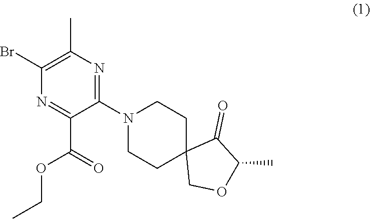 [SMILES: CCOC(=O)c1nc(Br)c(C)nc1N1CCC2(CC1)CO[C@@H](C)C2=O]

with the compound of Formula (A1):

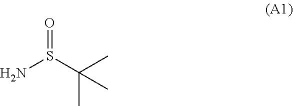

and a titanium alkoxide reagent to form the compound of Formula (2).

In some embodiments, the titanium alkoxide reagent is Ti(OCH₂CH₃)₄. In some embodiments, the titanium alkoxide reagent is Ti(OCH(CH₃)₂)₄.

In some embodiments, the reaction of the compound of Formula (1) with the compound of Formula (A1) is carried out using an aprotic solvent. In some embodiments, the aprotic solvent is tetrahydrofuran, 2-methyltetrahydrofuran, methylcyclohexane, hexanes, cyclopentyl methyl ether, acetonitrile, 1,4-dioxane, toluene, dichloromethane, dichloroethane, or chloroform. In some embodiments, the aprotic solvent is 2-methyltetrahydrofuran.

In some embodiments, the reaction of the compound of Formula (1) with the compound of Formula (A1) is carried out at a temperature of about 70-90° C. In some embodiments, the reaction temperature is about 80° C. In some embodiments, the reaction of the compound of Formula (1) with the compound of Formula (A1) is carried out at the reflux temperature of the solvent. For example, the reaction of the compound of Formula (1) with the compound of Formula (A1) can be carried out at 100° C. using methylcyclohexane as solvent.

In some embodiments, the reaction of the compound of Formula (1) with the compound of Formula (A1) and the titanium alkoxide reagent provides the compound of Formula (2) as a mixture of the compounds of Formula (2a) and Formula (2b). In some embodiments, the mixture comprises about 50% or more of the compound of Formula (2a), and about 50% or less of the compound of Formula (2b). In some embodiments, the mixture comprises about 50-99% of the compound of Formula (2a), such as about 50%, 55%, 60%, 65%, 70%, 75%, 80%, 85%, 90%, 95%, or 99% of the compound of Formula (2a), and about 1-50% of the compound of Formula (2b), such as about 1%, 5%, 10%, 15%, 20%, 25%, 30%, 35%, 40%, 45%, or 50% of the compound of Formula (2b). In some embodiments, the mixture comprises about 80-99% of the compound of Formula (2a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (2a), and about 1-20% of the compound of Formula (2b), such as about 1%, 2%, 3%, 4%, 5%, 6%, 7%, 8%, 9%, 10%1, 1%1, 2%1, 3%1, 14%, 15%, 6%1, 7%1, 8%1, 19%, or 20% of the compound of Formula (2b). In some embodiments, the mixture comprises about 90% or more of the compound of Formula (2a). In some embodiments, the mixture comprises about 99% of the compound of Formula (2a). In some embodiments, the mixture comprises about 99% of the compound of Formula (2a), and about 1% of the compound of Formula (2b); about 98% of the compound of Formula (2a), and about 2% of the compound of Formula (2b); about 97% of the compound of Formula (2a), and about 3% of the compound of Formula (2b); about 96% of the compound of Formula (2a), and about 4% of the compound of Formula (2b); about 95% of the compound of Formula (2a), and about 5% of the compound of Formula (2b); about 94% of the compound of Formula (2a), and about 6% of the compound of Formula (2b); about 93% of the compound of Formula (2a), and about 7% of the compound of Formula (2b); about 92% of the compound of Formula (2a), and about 8% of the compound of Formula (2b); about 91% of the compound of Formula (2a), and about 9% of the compound of Formula (2b); or about 90% of the compound of Formula (2a), and about 10% of the compound of Formula (2b).

In some embodiments, the compound of Formula (A1) is the compound of Formula (A1a):

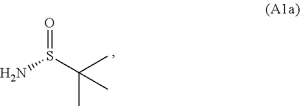

and the compound of Formula (2) is the compound of Formula (2a):

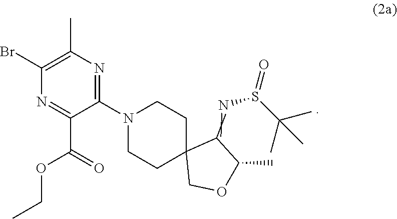 [SMILES: CCOC(=O)c1nc(Br)c(C)nc1N1CCC2(CC1)CO[C@@H](C)C2=N[S@](=O)C(C)(C)C]

In other embodiments, the compound of Formula (A1) is the compound of Formula (A1b):

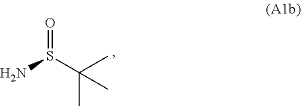

and the compound of Formula (2) is the compound of Formula (2b):

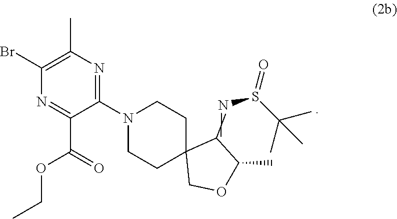 [SMILES: CCOC(=O)c1nc(Br)c(C)nc1N1CCC2(CC1)CO[C@@H](C)C2=N[S@@](=O)C(C)(C)C]

### Compound of Formula (1)

Also provided herein is a compound of Formula (1):

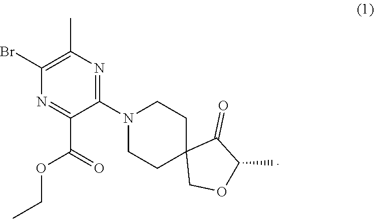 [SMILES: CCOC(=O)c1nc(Br)c(C)nc1N1CCC2(CC1)CO[C@@H](C)C2=O]

Although the compound of Formula (1) is shown in the present disclosure as an ethyl ester, other alkyl esters such as methyl, propyl (e.g., n-propyl or isopropyl), or butyl (e.g., n-butyl or tert-butyl) may alternatively be used. In some variations wherein an alternate alkyl ester (such as methyl, propyl, or butyl) of the compound of Formula (1) is used, it follows that the compound of Formula (2) will similarly be provided as the alternate alkyl ester (methyl, propyl, or butyl).

In yet another aspect, provided herein is a method of preparing a compound of Formula (1):

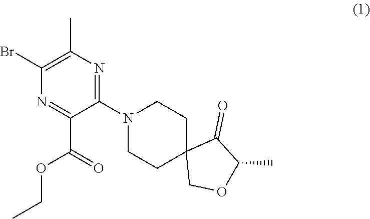 [SMILES: CCOC(=O)c1nc(Br)c(C)nc1N1CCC2(CC1)CO[C@@H](C)C2=O]

comprising reacting a compound of Formula (II):

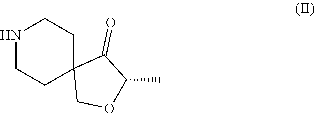

or a salt thereof,
with the compound of Formula (IV):

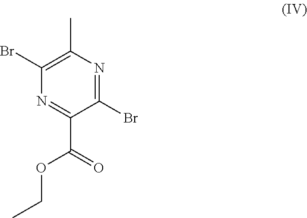

to form the compound of Formula (1).

In some embodiments, the compound of Formula (II) is provided as a salt. In some embodiments, the salt of the compound of Formula (II) is an HCl, HBr, trifluoroacetic acid, methanesulfonic acid, or H₂SO₄ salt. In some embodiments, the salt of the compound of Formula (II) is an HCl salt.

In some embodiments, the reaction of the compound of Formula (II) with the compound of Formula (IV) further comprises a base. In some embodiments, the base is K₂CO₃, Na₂CO₃, or NaHCO₃. In some embodiments, the base is K₂CO₃. In some embodiments, the base is a tertiary amine, such as a trialkylamine (for example, diisopropylethylamine or triethylamine). In some embodiments, the base is a trialkylamine.

In some embodiments, the reaction of the compound of Formula (II) with the compound of Formula (IV) is carried out using an aprotic solvent. In some embodiments, the aprotic solvent is tetrahydrofuran, 2-methyltetrahydrofuran, acetonitrile, 1,4-dioxane, toluene, dimethylsulfoxide, dimethylacetamide, N-methyl-2-pyrrolidone, dichloromethane, dichloroethane, or chloroform. In some embodiments, the aprotic solvent is dichloromethane.

In some embodiments, the reaction of the compound of Formula (II) with the compound of Formula (IV) is carried out at a temperature of about 30-50° C. In some embodiments, the reaction temperature is about 40° C. In some embodiments, the reaction is carried out at a temperature of about 10° C. to the reflux temperature of the solvent. In some embodiments, the reaction is carried out at the reflux temperature of the solvent. In some embodiments, the reaction is carried out at a temperature of about 30-180° C. In some embodiments, the reaction is carried out at a temperature of about 30-120° C. In some embodiments, the reaction is carried out at a temperature of about 30-150° C.

### Compound of Formula (H)

In one aspect, provided herein is a method of preparing a compound of Formula (II)

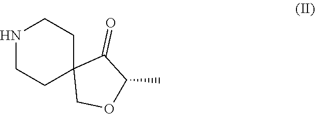

or a salt thereof,
comprising reacting a compound of Formula (I):

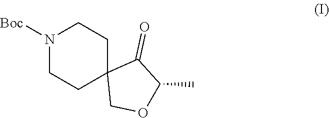 [SMILES: C[C@@H]1OCC2(CCN(C(=O)OC(C)(C)C)CC2)C1=O]

with an acid to form the compound of Formula (II) or a salt thereof.

In some embodiments, the compound of Formula (II) is provided as a salt. In some embodiments, the salt of Formula (II) is an HCl, HBr, trifluoroacetic acid, methanesulfonic acid, or H₂SO₄ salt. In some embodiments, the salt of Formula (II) is an HCl salt.

In some embodiments, the acid is HCl, HBr, methanesulfonic acid, trifluoroacetic acid, or H₂SO₄. In some embodiments, the acid is HCl. In some embodiments, HCl is generated in situ by reaction of acetyl chloride, trimethylsilyl chloride, or AlCl₃ with an alcohol, such as methanol or ethanol.

In some embodiments, the reaction of the compound of Formula (I) with the acid is carried out using an aprotic solvent. In some embodiments, the aprotic solvent is acetone, ethyl acetate, isopropyl acetate, tert-butyl acetate, 1,4-dioxane, tetrahydrofuran, 2-methyltetrahydrofuran, dichloromethane, or acetonitrile. In some embodiments, the aprotic solvent is acetone. In some embodiments, the aprotic solvent is an acetate, such as tert-butyl acetate, isopropyl acetate, or ethyl acetate. In some embodiments, the aprotic solvent contains an organic acid, such as trifluoroacetic acid. In some embodiments, the aprotic solvent contains an inorganic acid, such as HCl or HBr. In some embodiments, the reaction of the compound of Formula (I) with the acid is carried out using a protic solvent. In some embodiments, the protic solvent is an alcohol, such as isopropanol, ethanol, or methanol. In some embodiments, the reaction of the compound of Formula (I) with the acid is carried out using a mixture of an aprotic solvent and a protic solvent. In some embodiments, the reaction of the compound of Formula (I) with the acid is carried out using a mixture of an aprotic solvent, such as acetone, ethyl acetate, isopropyl acetate, tert-butyl acetate, 1,4-dioxane, tetrahydrofuran, 2-methyltetrahydrofuran, dichloromethane, or acetonitrile, and an alcohol, such as isopropanol, ethanol, or methanol. In some embodiments, the reaction of the compound of Formula (I) with the acid is carried out using a mixture of an acetate, such as tert-butyl acetate, isopropyl acetate, or ethyl acetate, and an alcohol, such as isopropanol, ethanol, or methanol. In some embodiments, the reaction of the compound of Formula (I) with the acid is carried out using a mixture of isopropyl acetate and isopropanol.

In some embodiments, the compound of Formula (I) is generated and directly used in situ.

### Compound of Formula (IV)

In one aspect, provided herein is a method of preparing a compound of Formula (IV):

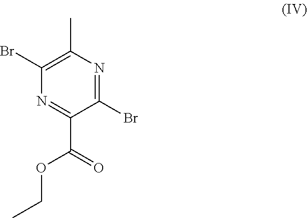

comprising reacting a compound of Formula (III):

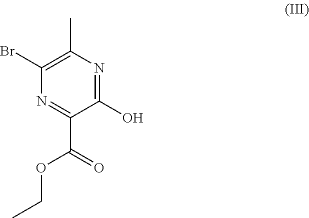

with POBr₃ to form the compound of Formula (IV).

Although the compound of Formula (III) and the compound of Formula (IV) are shown in the present disclosure as ethyl esters, other alkyl esters such as methyl, propyl (e.g., n-propyl or isopropyl), or butyl (e.g., n-butyl or tert-butyl) may alternatively be used. In some variations wherein an alternate alkyl ester (such as methyl, propyl, or butyl) of the compound of Formula (III) is used, it follows that the compound of Formula (IV) will similarly be provided as the alternate alkyl ester (methyl, propyl, or butyl). Further, it follows that wherein an alternate alkyl ester (such as methyl, propyl, or butyl) of the compounds of Formula (III) and (IV) are used, the compounds of Formula (1) and (2) will similarly be provided as the alternate alkyl esters (methyl, propyl, or butyl).

In some embodiments, the reaction of the compound of Formula (III) with POBr₃ is carried out using an aprotic solvent. In some embodiments, the aprotic solvent is tetrahydrofuran, 2-methyltetrahydrofuran, acetonitrile, 1,4-dioxane, toluene, xylene, dichloromethane, dichloroethane, or chloroform. In some embodiments, the aprotic solvent is dichloromethane.

In some embodiments, the reaction of the compound of Formula (III) with POBr₃ is carried out at a temperature of about 70-90° C. In some embodiments, the reaction temperature is about 80° C. In some embodiments, the reaction of the compound of Formula (III) with POBr₃ is carried out at a temperature of about 30-40° C. In some embodiments, the reaction of the compound of Formula (III) with POBr₃ is carried out at the reflux temperature of solvent. In some embodiments, the reaction temperature is about 110 or 120° C. In some embodiments, the reaction temperature is about 30 or 40° C.

In some embodiments, the reaction of the compound of Formula (III) with POBr₃ further comprises dimethylformamide as a catalyst.

Also provided herein is a method of preparing the compound of Formula (IV) comprising reacting the compound of Formula (III) with N-bromo-succinimide and triphenylphosphine to form the compound of Formula (IV). In some embodiments, the reaction of the compound of Formula (III) with N-bromo-succinimide and triphenylphosphine is carried out using an ether, such as 1,4-dioxane, as solvent. In some embodiments, the reaction of the compound of Formula (III) with N-bromo-succinimide and triphenylphosphine is carried out at a temperature of about 25-100° C. In some embodiments, the reaction is carried out at a temperature of about 100° C. In some embodiments, the reaction is carried out at the reflux temperature of the solvent.

### Compound of Formula (VI)

In still a further aspect, provided herein is a method of preparing a compound of Formula (VI):

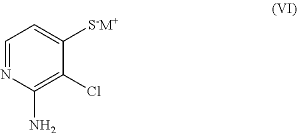

wherein M⁺ is Li⁺, Na⁺, or K⁺,
comprising reacting a compound of Formula (V):

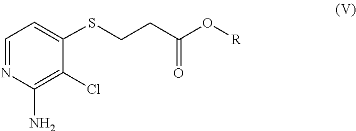

wherein R is C₁-C₁₂ alkyl,
with a base to form the compound of Formula (VI).

In some embodiments, R is C₁-C₁₂ alkyl. In some embodiments, R is C₁-C₁₀ alkyl. In some embodiments, R is C₁-C₆ alkyl. In some embodiments, R is C₁-C₃ alkyl. In some embodiments, R is methyl, ethyl, or propyl. In some embodiments, R is ethyl. In some embodiments, R is C₁-C₈ alkyl. In some embodiments, R is 1-ethylhexyl, 2-ethylhexyl, 3-ethylhexyl, 4-ethylhexyl, or 5-ethylhexyl. In some embodiments, R is 2-ethylhexyl and the compound of Formula (V) is the compound of Formula (Va):

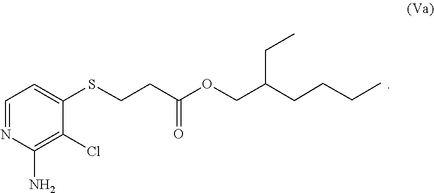 [SMILES: CCCCC(CC)COC(=O)CCSc1ccnc(N)c1Cl]

In some embodiments, the base is NaOH, KOH, LiGH, KOCH₃, NaOCH₃, LiOCH₃, KOCH₂CH₃, NaOCH₂CH₃, LiOCH₂CH₃, KO(tert-butyl), NaO(tert-butyl), or LiO(tert-butyl). In some embodiments, the base is KOCH₂CH₃.

In some embodiments, the reaction of the compound of Formula (V) with the base is carried out using an aprotic solvent. In some embodiments, the aprotic solvent is tetrahydrofuran, 2-methyltetrahydrofuran, acetonitrile, 1,4-dioxane, toluene, methyl tert-butyl ether, or xylene. In some embodiments, the aprotic solvent is 2-methyltetrahydrofuran. In some embodiments, the reaction of the compound of Formula (V) with the base is carried out using a mixture of an alcohol and an aprotic solvent. In some embodiments, the alcohol is ethanol or methanol. In some embodiments, the reaction of the compound of Formula (V) with the base is carried out using a mixture of 2-methyltetrahydrofuran and an alcohol, such as methanol, ethanol, or isopropanol. In some embodiments, the alcohol is ethanol. In some embodiments, the reaction of the compound of Formula (V) with the base is carried out using a protic solvent such as an alcohol, for example, methanol or ethanol.

In some embodiments, the reaction of the compound of Formula (V) with the base is carried out at a temperature of about 15-25° C. In some embodiments, the reaction temperature is about 22° C. In some embodiments, the reaction of the compound of Formula (V) with the base is carried out at a temperature of about 0-50° C. In some embodiments, the reaction of the compound of Formula (V) with the base is carried out at a temperature of about 10-30° C. In some embodiments, the reaction of the compound of Formula (V) with the base is carried out at the reflux temperature of the solvent. In some embodiments, the reaction temperature is about 10-120° C.

### Compound of Formula (7)

Also provided herein is a method of preparing a compound of Formula (7):

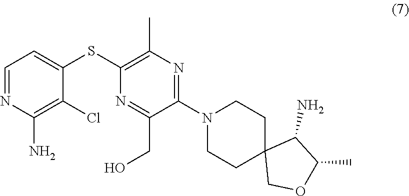 [SMILES: Cc1nc(N2CCC3(CC2)CO[C@@H](C)[C@H]3N)c(CO)nc1Sc1ccnc(N)c1Cl]

or a salt thereof,
comprising reacting the compound of Formula (6)

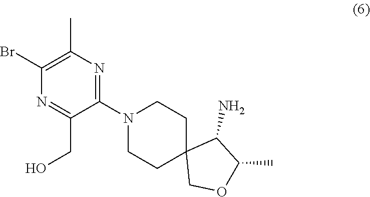 [SMILES: Cc1nc(N2CCC3(CC2)CO[C@@H](C)[C@H]3N)c(CO)nc1Br]

or a salt thereof, prepared according to any of the methods disclosed herein, with a compound of Formula (VI):

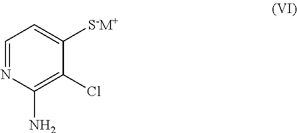

wherein M⁺ is Li⁺, Na⁺, or K⁺,
to form the compound of Formula (7) or a salt thereof.

In some embodiments, M⁺ is Li⁺. In some embodiments, M⁺ is Na⁺. In some embodiments, M⁺ is K⁺ and the compound of Formula (VI) is a compound of Formula (VIa):

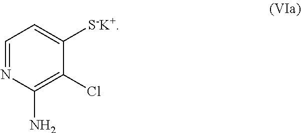

In some embodiments, the reaction of the compound of Formula (6), or a salt thereof, with the compound of Formula (VI) further comprises a copper salt. In some embodiments, the copper salt is a copper (I) salt. In other embodiments, the copper salt is a copper (II) salt. In some embodiments, the copper salt is CuI. In some embodiments, the copper salt is CuBr. In some embodiments, the copper salt is copper acetate hydrate (i.e., Cu(CO₂CH₃)₂·xH₂O). In some embodiments, the copper salt comprises imidazole. In other embodiments, the reaction of the compound of Formula (6), or a salt thereof, with the compound of Formula (VI) is carried out in the absence of a copper salt.

In some embodiments, the reaction of the compound of Formula (6), or a salt thereof, with the compound of Formula (VI) is carried out using an aprotic solvent. In some embodiments, the aprotic solvent is tetrahydrofuran, 2-methyltetrahydrofuran, acetonitrile, butyronitrile, sulfolane, dimethylformamide, N-methyl-2-pyrrolidone, dimethylacetamide, morpholine, 1,4-dioxane, ethylene glycol, toluene, pyridine, dichloromethane, dichloroethane, or chloroform. In some embodiments, the aprotic solvent is pyridine. In some embodiments, the aprotic solvent is an alkylene glycol, such as methylene glycol, ethylene glycol, or propylene glycol. In some embodiments, the reaction of the compound of Formula (6), or a salt thereof, with the compound of Formula (VI) is carried out using a mixture of an aprotic solvent and an alcohol, such as methanol, ethanol, or isopropanol. In some embodiments, the reaction of the compound of Formula (6), or a salt thereof, with the compound of Formula (VI) is carried out using a mixture of an alkylene glycol, such as methylene glycol, ethylene glycol, or propylene glycol, and an alcohol, such as methanol, ethanol, or isopropanol. In some embodiments, the reaction of the compound of Formula (6), or a salt thereof, with the compound of Formula (VI) is carried out using a mixture of ethylene glycol and isopropanol.

In some embodiments, the reaction of the compound of Formula (6), or a salt thereof, with the compound of Formula (VI) is carried out at a temperature of about 50-130° C. In some embodiments, the reaction temperature is about 80° C. In some embodiments, the reaction of the compound of Formula (6), or a salt thereof, with the compound of Formula (VI) is carried out at a temperature of about 100-130° C. In some embodiments, the reaction temperature is about 115° C. In some embodiments, the reaction of the compound of Formula (6), or a salt thereof, with the compound of Formula (VI) is carried out at reflux temperature of the solvent.

In some embodiments, the reaction of the compound of Formula (6), or a salt thereof, with the compound of Formula (VI) is carried out at an elevated pressure. In some embodiments, the pressure of the reaction is from about 1-10 bar (about 14.5-145 psi).

In some embodiments, the reaction of the compound of Formula (6), or a salt thereof, with the compound of Formula (VI) is carried out using flow chemistry. In some embodiments, the flow chemistry is performed at an elevated pressure, such as from about 1-10 bar (about 14.5-145 psi).

In some embodiments, provided herein is a method for preparing the compound of Formula (7), or a salt thereof, according to Scheme 2.

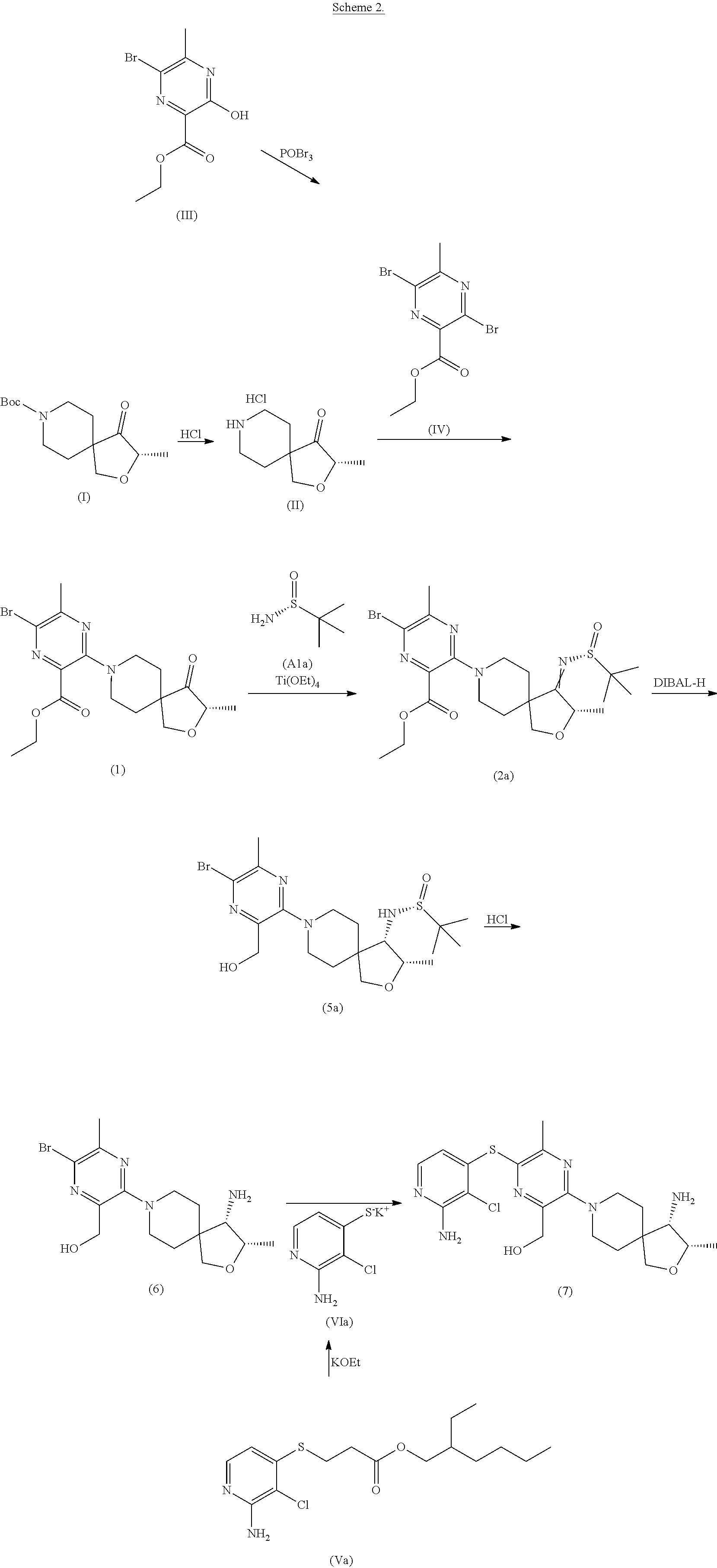

In some embodiments of the synthetic methods disclosed herein, the absolute configuration at the C₄ position on the spiro-cycle of the compound of Formula (7), the numbering of which is shown in Scheme 2, is promoted by the absolute stereochemistry at the sulfur atom of the compound of Formula (A1a). In some embodiments, the (S) absolute configuration at the C₄ position on the spiro-cycle of the compound of Formula (7) is promoted by the (R) absolute stereochemistry at the sulfur atom of the compound of Formula (A1a) and is carried forward in the compounds of Formula (2a), (5a), and (6).

In other embodiments, the preparation of the compound of Formula (7), or a salt thereof, comprises the reactions shown in Scheme 3.

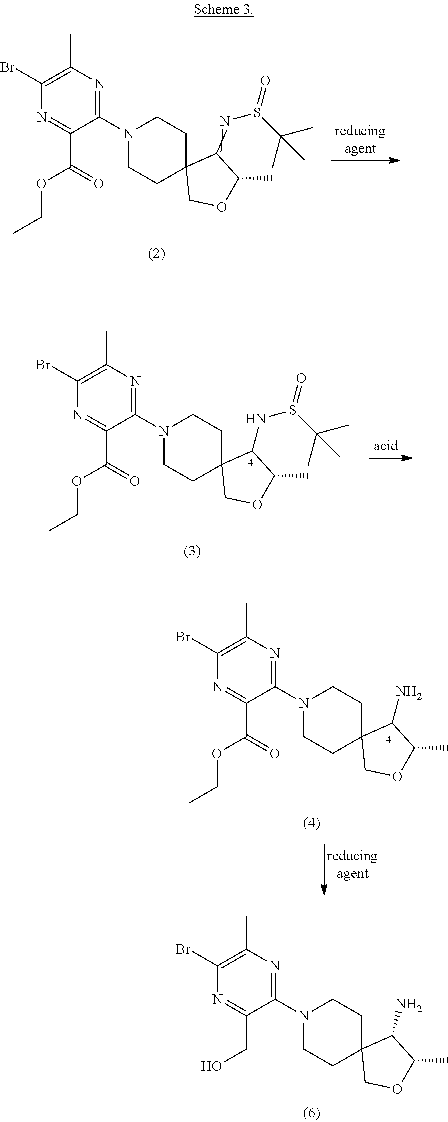 [SMILES: CCOC(=O)c1nc(Br)c(C)nc1N1CCC2(CC1)CO[C@@H](C)C2=NS(=O)C(C)(C)C ; CCOC(=O)c1nc(Br)c(C)nc1N1CCC2(CC1)CO[C@@H](C)C2NS(=O)C(C)(C)C ; CCOC(=O)c1nc(Br)c(C)nc1N1CCC2(CC1)CO[C@@H](C)C2N ; Cc1nc(N2CCC3(CC2)CO[C@@H](C)[C@H]3N)c(CO)nc1Br]

The sulfinyl imine compound of Formula (2) can be reduced using, for example DIBAL-H, to the sulfinamide compound of Formula (3), followed by treatment with an acid to form the amine compound of Formula (4). The ester moiety of the compound of Formula (4) can then be reduced using, for example DIBAL-H, to form the compound of Formula (6), and subsequently used to prepare the compound of Formula (7) as outlined in Schemes 1 and 2. In some embodiments, the compound of Formula (3) has (S) absolute configuration at C₄, and the compound of Formula (4) has (S) absolute configuration at C₄. In some embodiments, the compound of Formula (3) has the structure of Formula (3a), and the compound of Formula (4) has the structure of Formula (4a). Accordingly, in some embodiments, the preparation of the compound of Formula (7), or a salt thereof, comprises the reactions shown in Scheme 4.

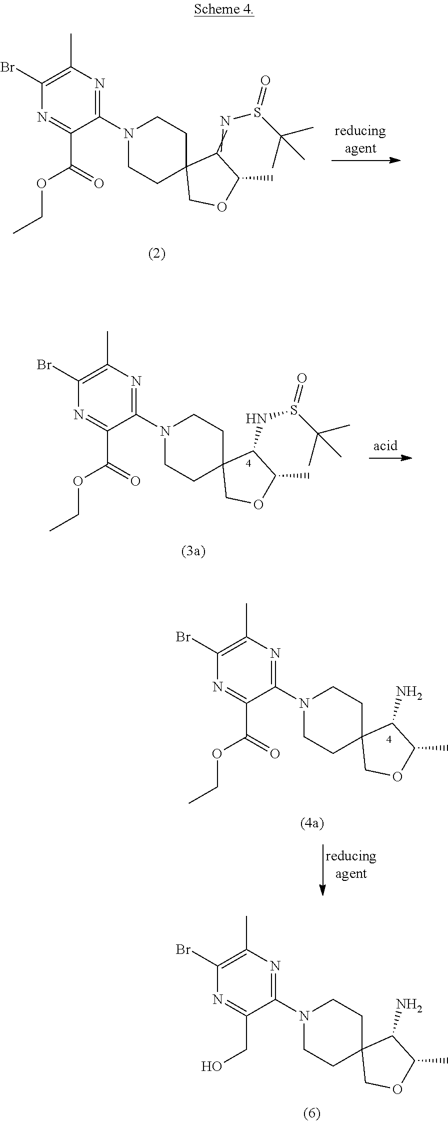 [SMILES: CCOC(=O)c1nc(Br)c(C)nc1N1CCC2(CC1)CO[C@@H](C)C2=NS(=O)C(C)(C)C ; CCOC(=O)c1nc(Br)c(C)nc1N1CCC2(CC1)CO[C@@H](C)[C@H]2N[S@](=O)C(C)(C)C ; CCOC(=O)c1nc(Br)c(C)nc1N1CCC2(CC1)CO[C@@H](C)[C@H]2N ; Cc1nc(N2CCC3(CC2)CO[C@@H](C)[C@H]3N)c(CO)nc1Br]

### Compound of Formula (3)

In one aspect, provided herein is a compound of Formula (3):

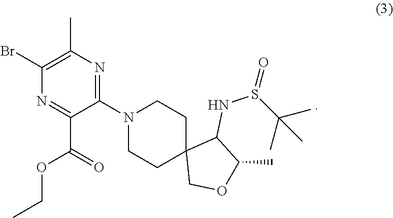 [SMILES: CCOC(=O)c1nc(Br)c(C)nc1N1CCC2(CC1)CO[C@@H](C)C2NS(=O)C(C)(C)C]

In some embodiments, the compound of Formula (3) has the structure of Formula (3a):

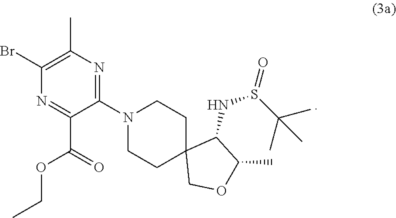 [SMILES: CCOC(=O)c1nc(Br)c(C)nc1N1CCC2(CC1)CO[C@@H](C)[C@H]2N[S@](=O)C(C)(C)C]

In other embodiments, the compound of Formula (3) has the structure of Formula (3b):

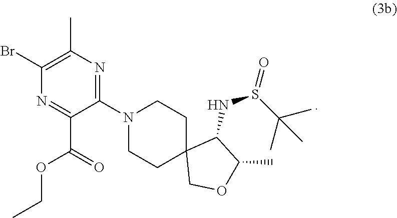 [SMILES: CCOC(=O)c1nc(Br)c(C)nc1N1CCC2(CC1)CO[C@@H](C)[C@H]2N[S@@](=O)C(C)(C)C]

Also provided herein is a method of preparing the compound of Formula (3)

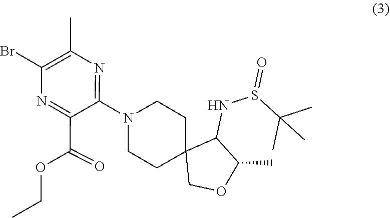 [SMILES: CCOC(=O)c1nc(Br)c(C)nc1N1CCC2(CC1)CO[C@@H](C)C2NS(=O)C(C)(C)C]

comprising reacting a compound of Formula (2):

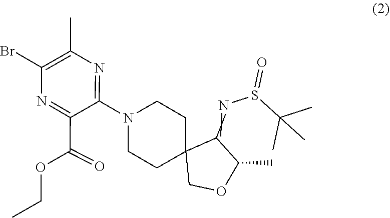 [SMILES: CCOC(=O)c1nc(Br)c(C)nc1N1CCC2(CC1)CO[C@@H](C)C2=NS(=O)C(C)(C)C]

with a reducing agent to form the compound of Formula (3).

In some embodiments, the reducing agent is an organoaluminum hydride, an organoborane hydride, or a borohydride reagent. In some embodiments, the reducing agent is diisobutylaluminum hydride (DIBAL-H), LiBHEt₃, L-selectride, N-selectride, K-selectride, sodium borohydride, lithium borohydride, or potassium borohydride. In some embodiments, the reducing agent is diisobutylaluminum hydride (DIBAL-H). In some embodiments, DIBAL-H is used as a neat liquid. In some embodiments, DIBAL-H is used as an organic solution of tetrahydrofuran, toluene, cyclohexane, heptane, or dichloromethane.

In some embodiments, the reaction of the compound of Formula (2) with the reducing agent is carried out using an aprotic solvent. In some embodiments, the aprotic solvent is tetrahydrofuran, 2-methyltetrahydrofuran, acetonitrile, 1,4-dioxane, toluene, dichloromethane, dichloroethane, chloroform, or a mixture thereof. In some embodiments, the aprotic solvent is 2-methyltetrahydrofuran or toluene. In some embodiments, the aprotic solvent is 2-methyltetrahydrofuran. In some embodiments, the aprotic solvent is toluene.

In some embodiments, the reaction of the compound of Formula (2) with the reducing agent provides the compound of Formula (3) as a mixture of the compounds of Formula (3a) and Formula (3b). In some embodiments, the mixture comprises about 50% or more of the compound of Formula (3a), and about 50% or less of the compound of Formula (3b). In some embodiments, the mixture comprises about 50-99% of the compound of Formula (3a), such as about 50%, 55%, 60%, 65%, 70%, 75%, 80%, 85%, 90%, 95%, or 99% of the compound of Formula (3a), and about 1-50% of the compound of Formula (3b), such as about 1%, 5%, 10%, 15%, 20%, 25%, 30%, 35%, 40%, 45%, or 50% of the compound of Formula (3b). In some embodiments, the mixture comprises about 80-99% of the compound of Formula (3a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (3a), and about 1-20% of the compound of Formula (3b), such as about 1%, 2%, 3%, 4%, 5%, 6%, 7%, 8%, 9%, 10%, 11%, 12%, 13%, 14%, 15%, 16%, 17%, 18%, 19%, or 20% of the compound of Formula (3b). In some embodiments, the mixture comprises about 90% or more of the compound of Formula (3a). In some embodiments, the mixture comprises about 99% of the compound of Formula (3a). In some embodiments, the mixture comprises about 99% of the compound of Formula (3a), and about 1% of the compound of Formula (3b); about 98% of the compound of Formula (3a), and about 2% of the compound of Formula (3b); about 97% of the compound of Formula (3a), and about 3% of the compound of Formula (3b); about 96% of the compound of Formula (3a), and about 4% of the compound of Formula (3b); about 95% of the compound of Formula (3a), and about 5% of the compound of Formula (3b); about 94% of the compound of Formula (3a), and about 6% of the compound of Formula (3b); about 93% of the compound of Formula (3a), and about 7% of the compound of Formula (3b); about 92% of the compound of Formula (3a), and about 8% of the compound of Formula (3b); about 91% of the compound of Formula (3a), and about 9% of the compound of Formula (3b); or about 90% of the compound of Formula (3a), and about 10% of the compound of Formula (3b).

### Compound of Formula (4)

Also provided herein is a method of preparing a compound of Formula (4):

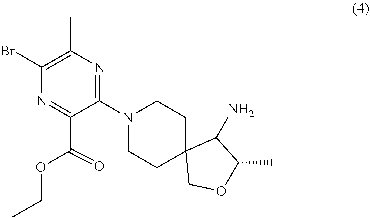 [SMILES: CCOC(=O)c1nc(Br)c(C)nc1N1CCC2(CC1)CO[C@@H](C)C2N]

or a salt thereof, comprising:
reacting a compound of Formula (3):

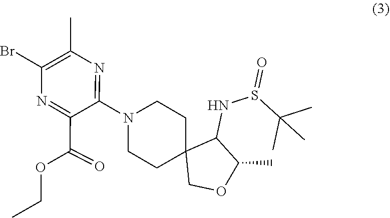 [SMILES: CCOC(=O)c1nc(Br)c(C)nc1N1CCC2(CC1)CO[C@@H](C)C2NS(=O)C(C)(C)C]

with an acid to form the compound of Formula (4) or a salt thereof.

In some embodiments, the compound of Formula (4) is a salt. In some embodiments, the salt of the compound of Formula (4) is an HCl, a methanesulfonic acid, or an HBr salt. In some embodiments, the salt of the compound of Formula (4) is an HCl salt. In some embodiments, the salt of the compound of Formula (4) is a methanesulfonic acid salt. In some embodiments, the salt of the compound of Formula (4) is an HBr salt.

In some embodiments, the acid used in the reaction is HCl, HBr, methanesulfonic acid, trifluoroacetic acid, or acetic acid. In some embodiments, the acid is HCl. In some embodiments, HCl is generated in situ by reaction of acetyl chloride, trimethylsilyl chloride, or AlCl₃ with an alcohol, such as methanol or ethanol.

In some embodiments, the reaction is carried out using an alcohol as solvent. In some embodiments, the alcohol is ethanol, methanol, or isopropanol. In some embodiments, the alcohol is ethanol. In some embodiments, the alcohol is methanol. In some embodiments, the reaction is carried out using a mixture of an ether and an alcohol, such as methanol or ethanol, as solvent. In some embodiments, the ether is 1,4-dioxane, tetrahydrofuran, 2-methyltetrahydrofuran, or diethyl ether. In some embodiments, the reaction is carried out using water as solvent. In some embodiments, the reaction is carried out using a mixture of water and alcohol, such as methanol or ethanol. In some embodiments, the reaction is carried out using a biphasic solvent system. In some embodiments, the biphasic solvent system is a mixture of water and 2-methyltetrahydrofuran. In some embodiments, the reaction is carried out using an acidic biphasic solvent system, such as HCl in a mixture of water and 2-methyltetrahydrofuran. In some embodiments, the reaction is carried out using acidic ethyl acetate as solvent, such as HCl in ethyl acetate. In some embodiments, the reaction is carried out using acidic dioxane as solvent, such as HCl in dioxane.

In some embodiments, the reaction is carried out at a temperature of about 0-25° C. In some embodiments, the reaction temperature is about 22° C. In some embodiments, the reaction is carried out at a temperature of about 0-100° C., such as about 35-90° C.

In some embodiments, the compound of Formula (3) has the structure of Formula (3a):

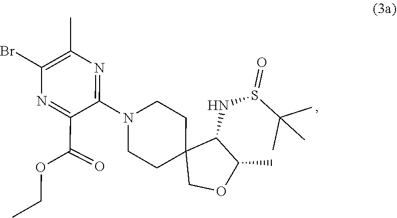 [SMILES: CCOC(=O)c1nc(Br)c(C)nc1N1CCC2(CC1)CO[C@@H](C)[C@H]2N[S@](=O)C(C)(C)C]

and the compound of Formula (4) has the structure of Formula (4a):

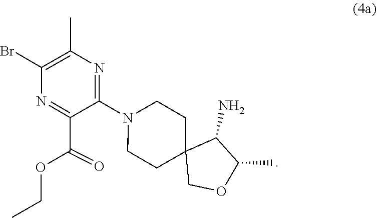 [SMILES: CCOC(=O)c1nc(Br)c(C)nc1N1CCC2(CC1)CO[C@@H](C)[C@H]2N]

In some embodiments, reaction of the compound of Formula (3) with the acid to provide the compound of Formula (4), or a salt thereof, comprises a mixture of the compounds of Formula (3a) and Formula (3b). In some embodiments, the mixture comprises about 50% or more of the compound of Formula (3a), and about 50% or less of the compound of Formula (3b). In some embodiments, the mixture comprises about 50-99% of the compound of Formula (3a), such as about 50%, 55%, 60%, 65%, 70%, 75%, 80%, 85%, 90%, 95%, or 99% of the compound of Formula (3a), and about 1-50% of the compound of Formula (3b), such as about 1%, 5%, 10%, 15%, 20%, 25%, 30%, 35%, 40%, 45%, or 50% of the compound of Formula (3b). In some embodiments, the mixture comprises about 80-99% of the compound of Formula (3a), such as about 80%, 81%, 82%, 83%, 84%, 85%, 86%, 87%, 88%, 89%, 90%, 91%, 92%, 93%, 94%, 95%, 96%, 97%, 98%, or 99% of the compound of Formula (3a), and about 1-20% of the compound of Formula (3b), such as about 1%, 2%, 3%, 4%, 5%, 6%, 7%, 8%, 9%, 10%, 11%, 12%, 13%, 14%, 15%, 16%, 17%, 18%, 19%, or 20% of the compound of Formula (3b).

In some embodiments, the mixture comprises at least about 80% of the compound of Formula (3a), and no more than 20% of the compound of Formula (3b). In some embodiments, the mixture comprises about 80% or more of the compound of Formula (3a), and 20% or less of the compound of Formula (3b). In some embodiments, the mixture comprises about 90% or more of the compound of Formula (3a). In some embodiments, the mixture comprises about 99% of the compound of Formula (3a). In some embodiments, the mixture comprises about 99% of the compound of Formula (3a), and about 1% of the compound of Formula (3b); about 98% of the compound of Formula (3a), and about 2% of the compound of Formula (3b); about 97% of the compound of Formula (3a), and about 3% of the compound of Formula (3b); about 96% of the compound of Formula (3a), and about 4% of the compound of Formula (3b); about 95% of the compound of Formula (3a), and about 5% of the compound of Formula (3b); about 94% of the compound of Formula (3a), and about 6% of the compound of Formula (3b); about 93% of the compound of Formula (3a), and about 7% of the compound of Formula (3b); about 92% of the compound of Formula (3a), and about 8% of the compound of Formula (3b); about 91% of the compound of Formula (3a), and about 9% of the compound of Formula (3b); or about 90% of the compound of Formula (3a), and about 10% of the compound of Formula (3b).

Although the synthetic reactions disclosed herein have been described using bromo as the leaving group for compounds of Formula (1), (2), (3), (4), (5), (6), (III), (IV), and variations thereof, it is understood that other leaving groups may be used to achieve similar chemical transformations. Suitable leaving groups that can be used include, without limitation, other halogens such as chloro or iodo, sulfonate esters such as tosylate or mesylate, and perfluoroalkylsulfonates such as triflate.

Accordingly, encompassed herein are the compounds of Formula (1-A), (2-A), (3-A), (4-A), (5-A), (6-A), (III-A), and (IV-A):

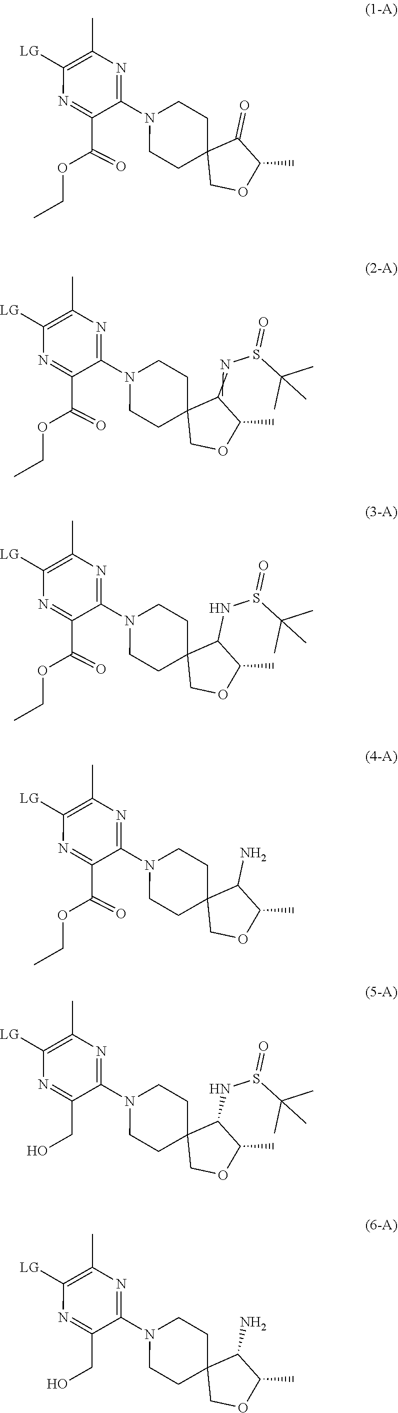

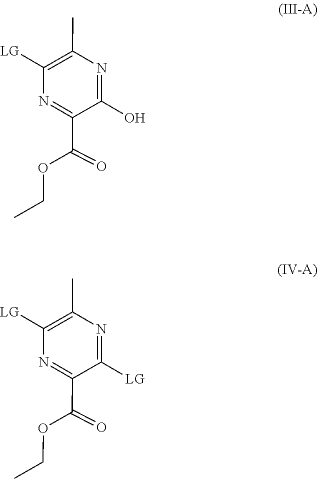

wherein LG is a suitable leaving group as described herein. Also encompassed herein is the use of the compounds of Formula (1-A), (2-A), (3-A), (4-A), (5-A), (6-A), (III-A), and (IV-A) in the synthesis of Compound (7) or a salt thereof.

Similarly, although the synthetic reactions disclosed herein have been described using tert-butyl attached to the sulfur atom for compounds of Formula (2), (3), (5), (A1), and variations thereof, it is understood that other groups (R′) may be used to achieve similar chemical transformations. Suitable R′ groups that can be used include, without limitation, other alkyl groups such as 2-methylbutyl, and aryl groups such as p-tolyl.

Accordingly, encompassed herein are the compounds of Formula (2-B), (3-B) (5-B), and (A1-B):

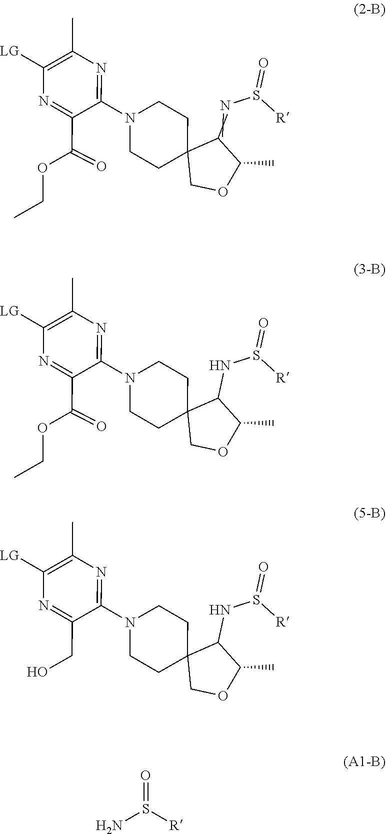

wherein LG is a suitable leaving group as described herein, and R′ is a suitable moiety as described herein. Also encompassed herein is the use of the compounds of Formula (2-B), (3-B) (5-B), and (A1-B) in the synthesis of Compound (7) or a salt thereof.

In addition, although the synthetic reactions disclosed herein have been described using Boc (tert-butyloxocarbonyl) as the protecting group for the compound of Formula (I), and variations thereof, it is understood that other protecting groups may be used to achieve similar chemical transformations. Suitable protecting groups that can be used include, without limitation, benzyl and benzyloxycarbonyl. In some embodiments, an acid-labile protecting group is used.

Accordingly, encompassed herein are compounds of Formula (I-C):

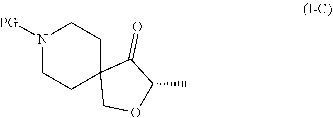

wherein PG is a suitable protecting group as described herein. Also encompassed herein is the use of the compounds of Formula (I-C) in the synthesis of Compound (7) or a salt thereof.

It will be understood that any of the compounds disclosed herein which exist in free base or acid form can be converted to their salts by treatment with the appropriate inorganic or organic base or acid by methods known to one skilled in the art. Similarly, salts of the compounds of the disclosure can be converted to their free base or acid form by standard techniques. Accordingly, where appropriate, salts of the compounds disclosed herein can be used in the synthetic methods in place of a described free base or acid form. Conversely, where appropriate, free base or acid forms of the compounds disclosed herein can be used in the synthetic methods in place of a described salt form.

### Compositions and Methods of Use

Compositions and uses of the compound of Formula (7), or a salt thereof, in treating or preventing a disease associated with SHP2 modulation in a subject in need thereof is described in U.S. Pat. No. 10,590,090, the disclosure of which is incorporated herein by reference.

Accordingly, in one aspect, provided herein is a method of treating a disease associated with SHP2 modulation in a subject in need thereof, comprising administering a therapeutically effective amount of the compound of Formula (7), or a salt thereof, prepared according to any of the methods disclosed herein, to the subject. In some embodiments, provided herein is a method of preventing a disease associated with SHP2 modulation in a subject in need thereof, comprising administering a therapeutically effective amount of the compound of Formula (7), or a salt thereof, prepared according to any of the methods disclosed herein, to the subject.

In some embodiments, provided herein is the use of the compound of Formula (7), or a salt thereof, prepared according to any of the methods disclosed herein, in the manufacture of a medicament for treating or preventing a disease associated with SHP2 modulation.

In some embodiments, provided herein is the use of the compound of Formula (7), or a salt thereof, prepared according to any of the methods disclosed herein, for treating or preventing a disease associated with SHP2 modulation in a subject in need thereof. In some embodiments, provided herein is the compound of Formula (7), or a salt thereof, prepared according to any of the methods disclosed herein, for treating or preventing a disease associated with SHP2 modulation in a subject in need thereof.

Non-limiting examples of a disease associated with SHP2 modulation include Noonan Syndrome, Leopard Syndrome, juvenile myelomonocytic leukemias, neuroblastoma, melanoma, acute myeloid leukemia, breast cancer, lung cancer, and colon cancer.

### Exemplary Embodiments

The present disclosure is further described by the following embodiments. The features of each of the embodiments are combinable with any of the other embodiments where appropriate and practical.

Embodiment 1. A method of preparing a compound of Formula (6):

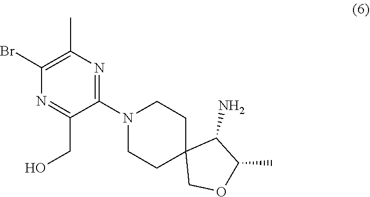 [SMILES: Cc1nc(N2CCC3(CC2)CO[C@@H](C)[C@H]3N)c(CO)nc1Br]

or a salt thereof, comprising:
reacting a compound of Formula (5):

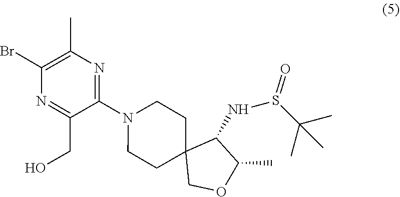 [SMILES: Cc1nc(N2CCC3(CC2)CO[C@@H](C)[C@H]3NS(=O)C(C)(C)C)c(CO)nc1Br]

with an acid to form the compound of Formula (6) or a salt thereof.

Embodiment 2. The method of embodiment 1, wherein the acid is HCl, HBr, methanesulfonic acid, or acetic acid.

Embodiment 3. The method of embodiment 1 or 2, wherein the acid is HCl.

Embodiment 4. The method of any one of embodiments 1-3, wherein the reaction is carried out using an alcohol, a mixture of water and an alcohol, or a mixture of water and 2-methyltetrahydrofuran as solvent.

Embodiment 5. The method of embodiment 4, wherein the alcohol is ethanol, methanol, or isopropanol.

Embodiment 6. The method of embodiment 4 or 5, wherein the alcohol is ethanol.

Embodiment 7. The method of embodiment 4, wherein the reaction is carried out using a mixture of water and 2-methyltetrahydrofuran.

Embodiment 8. The method of any one of embodiments 1-7, wherein the reaction is carried out at a temperature of about 0-25° C.

Embodiment 9. The method of embodiment 8, wherein the reaction is carried out at a temperature of about 22° C.

Embodiment 10. The method of any one of embodiments 1-9, wherein the compound of Formula (5) is the compound of Formula (5a):

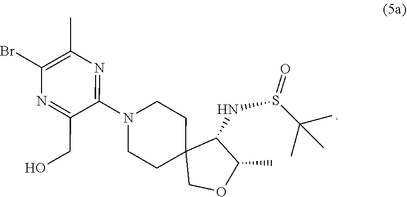 [SMILES: Cc1nc(N2CCC3(CC2)CO[C@@H](C)[C@H]3N[S@](=O)C(C)(C)C)c(CO)nc1Br]

Embodiment 11. A method of preparing a compound of Formula (6):

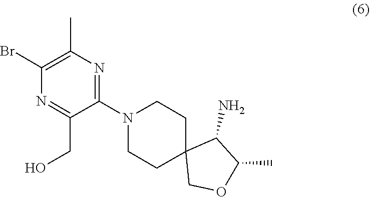 [SMILES: Cc1nc(N2CCC3(CC2)CO[C@@H](C)[C@H]3N)c(CO)nc1Br]

or a salt thereof, comprising:
reacting a compound of Formula (2):

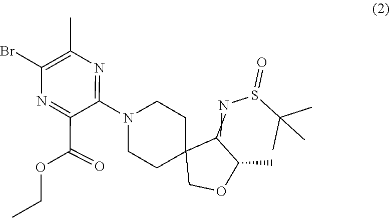 [SMILES: CCOC(=O)c1nc(Br)c(C)nc1N1CCC2(CC1)CO[C@@H](C)C2=NS(=O)C(C)(C)C]

with a reducing agent to form a compound of Formula (5):

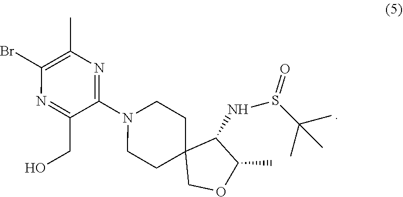 [SMILES: Cc1nc(N2CCC3(CC2)CO[C@@H](C)[C@H]3NS(=O)C(C)(C)C)c(CO)nc1Br]

Embodiment 12. The method of any one of embodiments 1-10, wherein the compound of Formula (5) is prepared by reacting a compound of Formula (2):

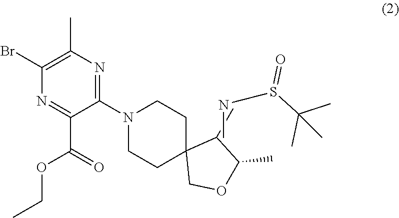

with a reducing agent to form the compound of Formula (5).

Embodiment 13. The method of embodiment 11 or 12, wherein the reducing agent is an organoaluminum hydride, an organoborane hydride, or a borohydride reagent.

Embodiment 14. The method of any one of embodiments 11-13, wherein the reducing agent is diisobutylaluminum hydride (DIBAL-H), LiBHEt₃, L-selectride, N-selectride, K-selectride, sodium borohydride, lithium borohydride, or potassium borohydride.

Embodiment 15. The method of any one of embodiments 11-14, wherein the reducing agent is diisobutylaluminum hydride (DIBAL-H).

Embodiment 16. The method of any one of embodiments 11-15, wherein the reaction of the compound of Formula (2) with the reducing agent is carried out using an aprotic solvent.

Embodiment 17. The method of embodiment 16, wherein the aprotic solvent is tetrahydrofuran, 2-methyltetrahydrofuran, acetonitrile, 1,4-dioxane, toluene, dichloromethane, dichloroethane, chloroform, or a mixture thereof.

Embodiment 18. The method of embodiment 16 or 17, wherein the aprotic solvent is 2-methyltetrahydrofuran.

Embodiment 19. The method of any one of embodiments 11-18, wherein the reaction of the compound of Formula (2) with the reducing agent is carried out at a temperature of about −30 to 30° C.

Embodiment 20. The method of embodiment 19, wherein the reaction of the compound of Formula (2) with the reducing agent is carried out at a temperature of about −20° C.

Embodiment 21. The method of any one of embodiments 11-20, wherein the compound of Formula (2) is the compound of Formula (2a):

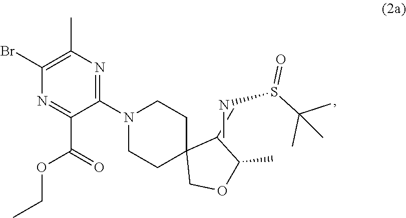

and the compound of Formula (5) is the compound of Formula (5a):

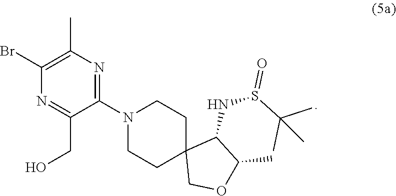 [SMILES: Cc1nc(N2CCC3(CC2)CO[C@@H](C)[C@H]3N[S@](=O)C(C)(C)C)c(CO)nc1Br]

Embodiment 22. A method of preparing a compound of Formula (6):

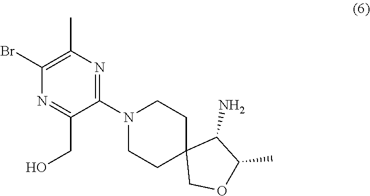 [SMILES: Cc1nc(N2CCC3(CC2)CO[C@@H](C)[C@H]3N)c(CO)nc1Br]

or a salt thereof, comprising:
reacting a compound of Formula (1):

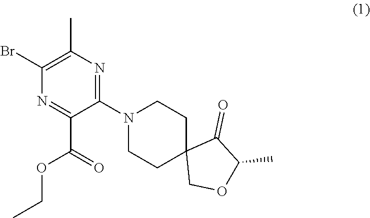 [SMILES: CCOC(=O)c1nc(Br)c(C)nc1N1CCC2(CC1)CO[C@@H](C)C2=O]

with a compound of Formula (A1):

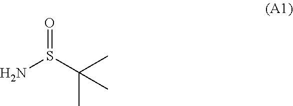

and a titanium alkoxide reagent to form a compound of Formula (2):

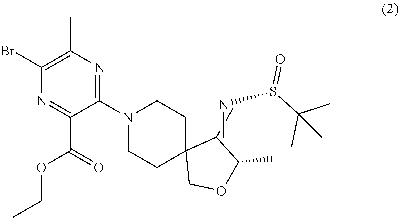

Embodiment 23. The method of any one of embodiments 11-21, wherein the compound of Formula (2) is prepared by reacting the compound of Formula (1):

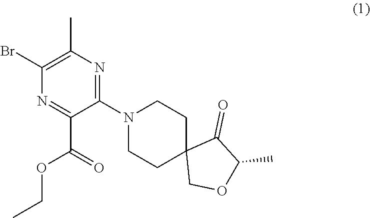 [SMILES: CCOC(=O)c1nc(Br)c(C)nc1N1CCC2(CC1)CO[C@@H](C)C2=O]

with the compound of Formula (A1):

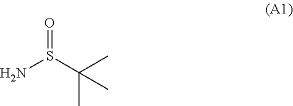

and a titanium alkoxide reagent to form the compound of Formula (2).

Embodiment 24. The method of embodiment 22 or 23, wherein the titanium alkoxide reagent is Ti(OCH₂CH₃)₄.

Embodiment 25. The method of any one of embodiments 22-24, wherein the reaction of the compound of Formula (1) with the compound of Formula (A1) is carried out using an aprotic solvent.

Embodiment 26. The method of embodiment 25, wherein the aprotic solvent used for the reaction of the compound of Formula (1) with the compound of Formula (A1) is tetrahydrofuran, 2-methyltetrahydrofuran, acetonitrile, 1,4-dioxane, toluene, dichloromethane, dichloroethane, or chloroform.

Embodiment 27. The method of embodiment 25 or 26, wherein the aprotic solvent used for the reaction of the compound of Formula (1) with the compound of Formula (A1) is 2-methyltetrahydrofuran.

Embodiment 28. The method of any one of embodiments 22-27, wherein the reaction of the compound of Formula (1) with the compound of Formula (A1) is carried out at a temperature of about 70-90° C.

Embodiment 29. The method of embodiment 28, wherein the reaction of the compound of Formula (1) with the compound of Formula (A1) is carried out at a temperature of about 80° C.

Embodiment 30. The method of any one of embodiments 22-29, wherein the compound of Formula (A1) is the compound of Formula (A1a):

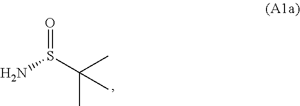

and the compound of Formula (2) is the compound of Formula (2a):

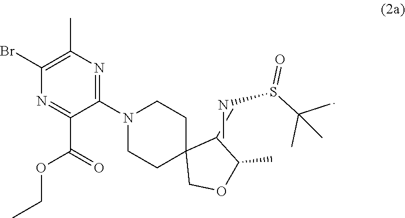

Embodiment 31. A method of preparing a compound of Formula (6):

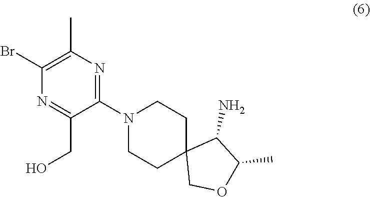 [SMILES: Cc1nc(N2CCC3(CC2)CO[C@@H](C)[C@H]3N)c(CO)nc1Br]

or a salt thereof, comprising:
reacting a compound of Formula (II):

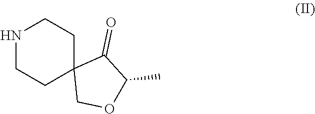

or a salt thereof,
with a compound of Formula (IV):

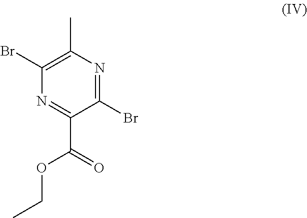

to form a compound of Formula (1):

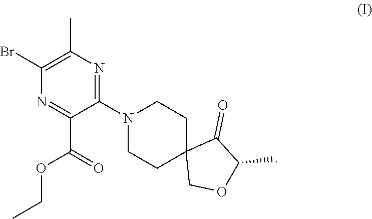 [SMILES: CCOC(=O)c1nc(Br)c(C)nc1N1CCC2(CC1)CO[C@@H](C)C2=O]

Embodiment 32. The method of any one of embodiments 22-30, wherein the compound of Formula (1) is prepared by reacting a compound of Formula (II):

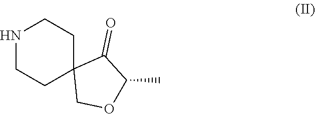

or a salt thereof,
with the compound of Formula (IV):

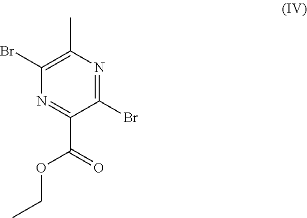

to form the compound of Formula (1).

Embodiment 33. The method of embodiment 31 or 32, comprising a salt of the compound of Formula (II).

Embodiment 34. The method of embodiment 33, wherein the salt of the compound of Formula (II) is an HCl, HBr, or methanesulfonic salt.

Embodiment 35. The method of embodiment 33 or 34, wherein the salt of the compound of Formula (II) is an HCl salt.

Embodiment 36. The method of any one of embodiments 31-35, wherein the reaction of the compound of Formula (II), or a salt thereof, with the compound of Formula (IV) further comprises a base.

Embodiment 37. The method of embodiment 36, wherein the base is K₂CO₃, Na₂CO₃, or NaHCO₃.

Embodiment 38. The method of embodiment 36 or 37, wherein the base is K₂CO₃.

Embodiment 39. The method of any one of embodiments 31-38, wherein the reaction of the compound of Formula (II), or a salt thereof, with the compound of Formula (IV) is carried out using an aprotic solvent.

Embodiment 40. The method of embodiment 39, wherein the aprotic solvent used for the reaction of the compound of Formula (II), or a salt thereof, with the compound of Formula (IV) is tetrahydrofuran, 2-methyltetrahydrofuran, acetonitrile, 1,4-dioxane, toluene, dichloromethane, dichloroethane, or chloroform.

Embodiment 41. The method of embodiment 39 or 40, wherein the aprotic solvent used for the reaction of the compound of Formula (II), or a salt thereof, with the compound of Formula (IV) is dichloromethane.

Embodiment 42. The method of any one of embodiments 31-41, wherein the reaction of the compound of Formula (II), or a salt thereof, with the compound of Formula (IV) is carried out at a temperature of about 30-120° C.

Embodiment 43. The method of embodiment 42, wherein the reaction of the compound of Formula (II), or a salt thereof, with the compound of Formula (IV) is carried out at a temperature of about 40° C.

Embodiment 44. A method of preparing a compound of Formula (6):

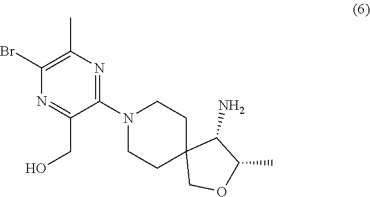 [SMILES: Cc1nc(N2CCC3(CC2)CO[C@@H](C)[C@H]3N)c(CO)nc1Br]

or a salt thereof, comprising:
reacting a compound of Formula (I):

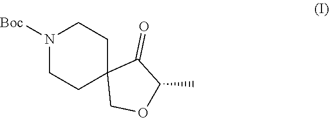 [SMILES: C[C@@H]1OCC2(CCN(C(=O)OC(C)(C)C)CC2)C1=O]

with an acid to form a compound of Formula (II):

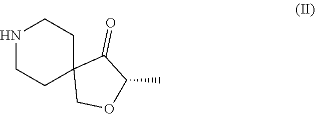

or a salt thereof.

Embodiment 45. The method of any one of embodiments 31-43, wherein the compound of Formula (II), or a salt thereof, is prepared by reacting a compound of Formula (I):

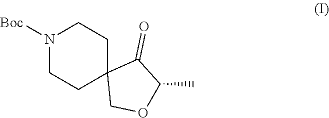 [SMILES: C[C@@H]1OCC2(CCN(C(=O)OC(C)(C)C)CC2)C1=O]

with an acid to form the compound of Formula (II) or a salt thereof.

Embodiment 46. The method of embodiment 44 or 45, wherein the acid used for the reaction of the compound of Formula (I) is HCl or HBr.

Embodiment 47. The method of any one of embodiments 44-46, wherein the acid used for the reaction of the compound of Formula (I) is HCl.

Embodiment 48. The method of any one of embodiments 44-47, wherein the reaction of the compound of Formula (I) with the acid is carried out using an aprotic solvent or a mixture of an aprotic solvent and an alcohol.

Embodiment 49. The method of embodiment 48, wherein the aprotic solvent used for the reaction of the compound of Formula (I) with the acid is acetone, tetrahydrofuran, 2-methyltetrahydrofuran, or acetonitrile.

Embodiment 50. The method of embodiment 48 or 49, wherein the aprotic solvent used for the reaction of the compound of Formula (I) with the acid is acetone.

Embodiment 51. The method of embodiment 48, wherein the reaction of the compound of Formula (I) with the acid is carried out using a mixture of an acetate and methanol, ethanol, or isopropanol.

Embodiment 52. The method of embodiment 51, wherein the reaction of the compound of Formula (I) with the acid is carried out using a mixture of isopropyl acetate and isopropanol.

Embodiment 53. A method of preparing a compound of Formula (6):

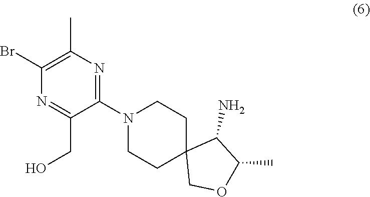 [SMILES: Cc1nc(N2CCC3(CC2)CO[C@@H](C)[C@H]3N)c(CO)nc1Br]

or a salt thereof, comprising:
reacting a compound of Formula (III):

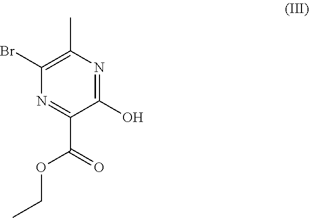

with POBr₃ to form a compound of Formula (IV):

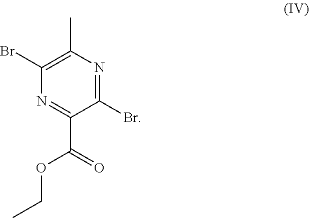

Embodiment 54. The method of any one of embodiments 31-52, wherein the compound of Formula (IV) is prepared by reacting a compound of Formula (III):

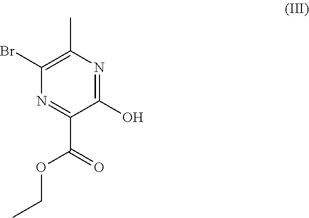

with POBr₃ to form the compound of Formula (IV).

Embodiment 55. The method of embodiment 53 or 54, wherein the reaction of the compound of Formula (III) with POBr₃ is carried out using an aprotic solvent.

Embodiment 56. The method of embodiment 55, wherein the aprotic solvent used for the reaction of the compound of Formula (III) with POBr₃ is tetrahydrofuran, 2-methyltetrahydrofuran, acetonitrile, 1,4-dioxane, toluene, dichloromethane, dichloroethane, or chloroform.

Embodiment 57. The method of embodiment 55 or 56, wherein the aprotic solvent used for the reaction of the compound of Formula (III) with POBr₃ is dichloroethane.

Embodiment 58. The method of any one of embodiments 53-57, wherein the reaction of the compound of Formula (III) with POBr₃ further comprises dimethylformamide as a catalyst.

Embodiment 59. The method of any one of embodiments 53-58, wherein the reaction of the compound of Formula (III) with POBr₃ is carried out at a temperature of about 70-90° C.

Embodiment 60. The method of embodiment 59, wherein the reaction of the compound of Formula (III) with POBr₃ is carried out at a temperature of about 80° C.

Embodiment 61. A method of preparing a compound of Formula (7):

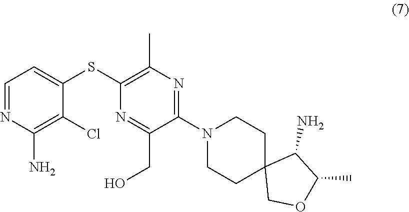 [SMILES: Cc1nc(N2CCC3(CC2)CO[C@@H](C)[C@H]3N)c(CO)nc1Sc1ccnc(N)c1Cl]

or a salt thereof, comprising reacting the compound of Formula (6), or a salt thereof, prepared according to any one of embodiments 1-60 with a compound of Formula (VI):

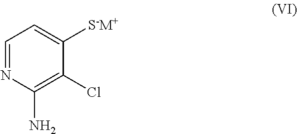

wherein M⁺ is Li⁺, Na⁺, or K⁺,
to form the compound of Formula (7) or a salt thereof.

Embodiment 62. The method of embodiment 61, wherein M⁺ is K⁺.

Embodiment 63. The method of embodiment 61 or 62, wherein the reaction of the compound of Formula (6), or a salt thereof, with the compound of Formula (VI) further comprises a copper salt.

Embodiment 64. The method of embodiment 63, wherein the copper salt is CuI or CuBr.

Embodiment 65. The method of any one of embodiments 61-64, wherein the reaction of the compound of Formula (6), or a salt thereof, with the compound of Formula (VI) is carried out using an aprotic solvent.

Embodiment 66. The method of embodiment 65, wherein the aprotic solvent used for the reaction of the compound of Formula (6), or a salt thereof, with the compound of Formula (VI) is tetrahydrofuran, 2-methyltetrahydrofuran, acetonitrile, 1,4-dioxane, toluene, pyridine, dichloromethane, dichloroethane, sulfolane, butyronitrile, dimethylsulfoxide, dimethylacetamide, or chloroform.

Embodiment 67. The method of embodiment 65 or 66, wherein the aprotic solvent used for the reaction of the compound of Formula (6), or a salt thereof, with the compound of Formula (VI) is pyridine.

Embodiment 68. The method of any one of embodiments 61-67, wherein the reaction of the compound of Formula (6), or a salt thereof, with the compound of Formula (VI) is carried out at a temperature of about 100-150° C.

Embodiment 69. The method of embodiment 68, wherein the reaction of the compound of Formula (6), or a salt thereof, with the compound of Formula (VI) is carried out at a temperature of about 115° C.

Embodiment 70. The method of any one of embodiments 61-69, wherein the compound of Formula (VI) is prepared by reacting a compound of Formula (V):

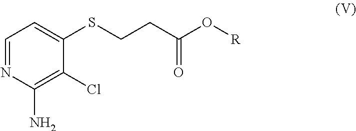

wherein R is C₁-C₁₂ alkyl,
with a base to form the compound of Formula (VI).

Embodiment 71. The method of embodiment 70, wherein the compound of Formula (V) is the compound of Formula (Va):

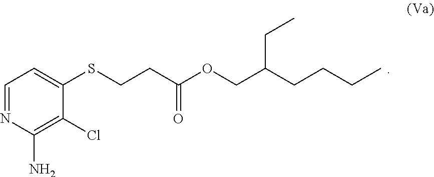 [SMILES: CCCCC(CC)COC(=O)CCSc1ccnc(N)c1Cl]

Embodiment 72. The method of embodiment 70 or 71, wherein the base used for the reaction of the compound of Formula (V) is NaOH, KOH, LiGH, KOCH₃, NaOCH₃, LiOCH₃, KOCH₂CH₃, NaOCH₂CH₃, LiOCH₂CH₃, NaO(tert-butyl), KO(tert-butyl), or LiO(tert-butyl).

Embodiment 73. The method of any one of embodiments 70-72, wherein the base used for the reaction of the compound of Formula (V) is KOCH₂CH₃.

Embodiment 74. The method of any one of embodiments 70-73, wherein the reaction of the compound of Formula (V) with the base is carried out using an aprotic solvent or a mixture of an aprotic solvent and an alcohol.

Embodiment 75. The method of embodiment 74, wherein the aprotic solvent used for the reaction of the compound of Formula (V) with the base is tetrahydrofuran, 2-methyltetrahydrofuran, acetonitrile, 1,4-dioxane, toluene, dichloromethane, dichloroethane, or chloroform.

Embodiment 76. The method of embodiment 74 or 75, wherein the aprotic solvent used for the reaction of the compound of Formula (V) with the base is 2-methyltetrahydrofuran.

Embodiment 77. The method of any one of embodiments 74-76, wherein the alcohol used for the reaction of the compound of Formula (V) with the base is methanol, ethanol, or isopropanol.

Embodiment 78. The method of any one of embodiments 70-77, wherein the reaction of the compound of Formula (V) with the base is carried out at a temperature of about 15-25° C.

Embodiment 79. The method of embodiment 78, wherein the reaction of the compound of Formula (V) with the base is carried out at a temperature of about 22° C.

Embodiment 80. A method of preparing a compound of Formula (7), or a salt thereof, comprising the following steps:

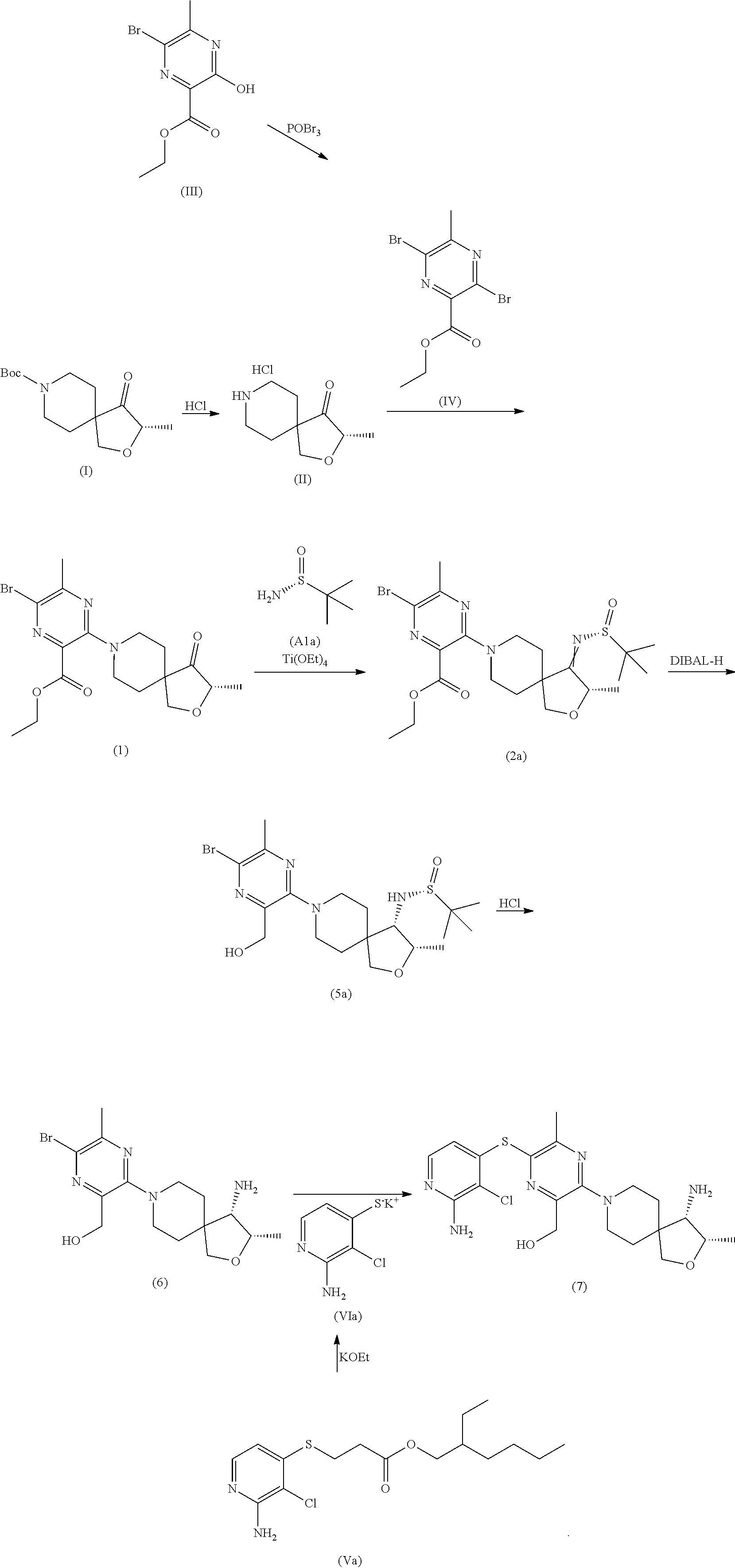

Embodiment 81. A method of preparing a compound of Formula (4):

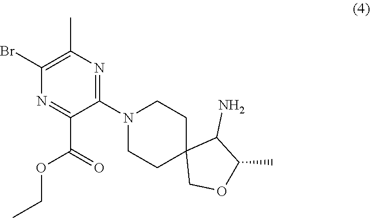 [SMILES: CCOC(=O)c1nc(Br)c(C)nc1N1CCC2(CC1)CO[C@@H](C)C2N]

or a salt thereof, comprising:
reacting a compound of Formula (3):

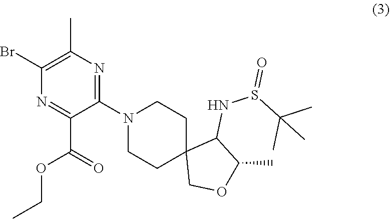 [SMILES: CCOC(=O)c1nc(Br)c(C)nc1N1CCC2(CC1)CO[C@@H](C)C2NS(=O)C(C)(C)C]

with an acid to form the compound of Formula (4) or a salt thereof.

Embodiment 82. The method of embodiment 81, wherein the compound of Formula (4) is a salt.

Embodiment 83. The method of embodiment 82, wherein the salt of the compound of Formula (4) is an HCl, HBr, or methanesulfonic acid salt.

Embodiment 84. The method of embodiment 81 or 82, wherein the salt of the compound of Formula (4) is an HCl salt.

Embodiment 85. The method of any one of embodiments 81-84, wherein the acid used for the reaction of the compound of Formula (3) is HCl, HBr, methanesulfonic acid, or acetic acid.

Embodiment 86. The method of any one of embodiments 81-85, wherein the acid used for the reaction of the compound of Formula (3) is HCl.

Embodiment 87. The method of any one of embodiments 81-86, wherein the reaction of the compound of Formula (3) and the acid is carried out using an alcohol as solvent.

Embodiment 88. The method of embodiment 87, wherein the alcohol used for the reaction of the compound of Formula (3) and the acid is ethanol, methanol, or isopropanol.

Embodiment 89. The method of embodiment 87 or 88, wherein the alcohol used for the reaction of the compound of Formula (3) and the acid is ethanol.

Embodiment 90. The method of any one of embodiments 81-89, wherein the reaction of the compound of Formula (3) and the acid is carried out at a temperature of about 0-25° C.

Embodiment 91. The method of embodiment 90, wherein the reaction of the compound of Formula (3) and the acid is carried out at a temperature of about 22° C.

Embodiment 92. The method of any one of embodiments 81-91, wherein the compound of Formula (3) is prepared by reacting a compound of Formula (2):

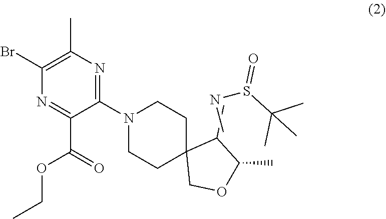

with a reducing agent to form the compound of Formula (3).

Embodiment 93. The method of embodiment 92, wherein the reducing agent used for the reaction of the compound of Formula (2) is an organoaluminum hydride, an organoborane hydride, or a borohydride reagent.

Embodiment 94. The method of embodiment 92 or 93, wherein the reducing agent used for the reaction of the compound of Formula (2) is diisobutylaluminum hydride (DIBAL-H), LiBHEt₃, L-selectride, N-selectride, K-selectride, lithium borohydride, sodium borohydride, or potassium borohydride.

Embodiment 95. The method of any one of embodiments 92-94, wherein the reducing agent used for the reaction of the compound of Formula (2) is diisobutylaluminum hydride (DIBAL-H).

Embodiment 96. The method of any one of embodiments 92-95, wherein the reaction of the compound of Formula (2) with the reducing agent is carried out using an aprotic solvent.

Embodiment 97. The method of embodiment 96, wherein the aprotic solvent used for the reaction of the compound of Formula (2) with the reducing agent is tetrahydrofuran, 2-methyltetrahydrofuran, acetonitrile, 1,4-dioxane, toluene, dichloromethane, dichloroethane, chloroform, or a mixture thereof.

Embodiment 98. The method of embodiment 96 or 97, wherein the aprotic solvent used for the reaction of the compound of Formula (2) with the reducing agent is 2-methyltetrahydrofuran or toluene.

Embodiment 99. The method of any one of embodiments 81-98, wherein the compound of Formula (3) is provided as a mixture of the compound of Formula (3a) and the compound of Formula (3b):

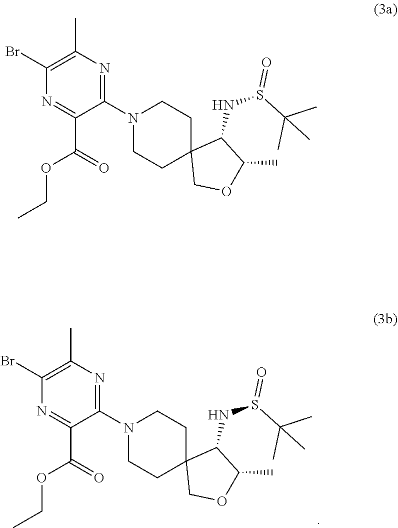 [SMILES: CCOC(=O)c1nc(Br)c(C)nc1N1CCC2(CC1)CO[C@@H](C)[C@H]2N[S@](=O)C(C)(C)C ; CCOC(=O)c1nc(Br)c(C)nc1N1CCC2(CC1)CO[C@@H](C)[C@H]2N[S@@](=O)C(C)(C)C]

Embodiment 100. The method of embodiment 99, wherein the mixture comprises at least about 80% of the compound of Formula (3a).

Embodiment 101. The method of embodiment 99 or 100, wherein the mixture comprises about 99% of the compound of Formula (3a).

Embodiment 102. A compound of Formula (1):

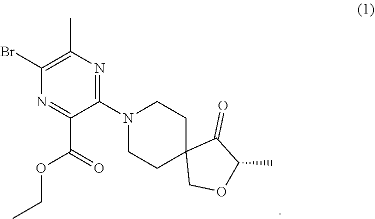 [SMILES: CCOC(=O)c1nc(Br)c(C)nc1N1CCC2(CC1)CO[C@@H](C)C2=O]

Embodiment 103. A compound of Formula (2):

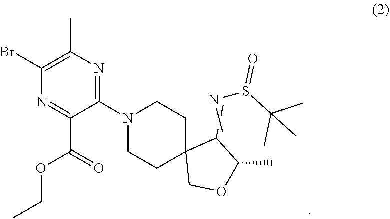

Embodiment 104. The compound of embodiment 103, which is of Formula (2a):

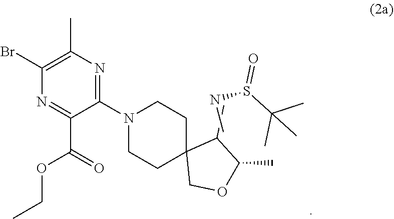

Embodiment 105. The compound of embodiment 103, which is of Formula (2b):

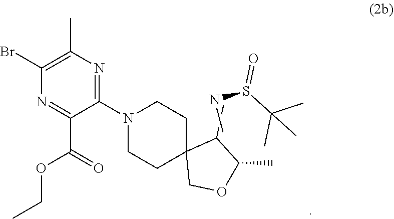

Embodiment 106. A compound of Formula (3):

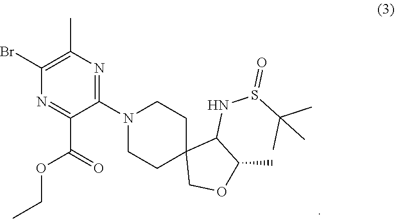 [SMILES: CCOC(=O)c1nc(Br)c(C)nc1N1CCC2(CC1)CO[C@@H](C)C2NS(=O)C(C)(C)C]

Embodiment 107. The compound of embodiment 106, which is of Formula (3a):

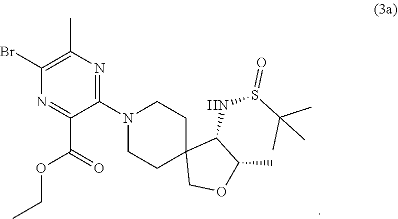 [SMILES: CCOC(=O)c1nc(Br)c(C)nc1N1CCC2(CC1)CO[C@@H](C)[C@H]2N[S@](=O)C(C)(C)C]

Embodiment 108. The compound of embodiment 106, which is of Formula (3b):

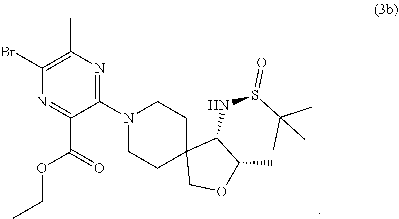 [SMILES: CCOC(=O)c1nc(Br)c(C)nc1N1CCC2(CC1)CO[C@@H](C)[C@H]2N[S@@](=O)C(C)(C)C]

Embodiment 109. The compound of embodiment 106, which is of Formula (3b′):

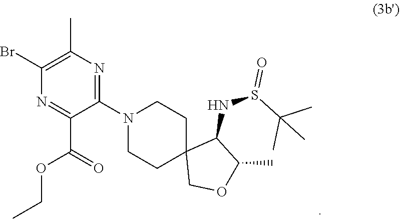 [SMILES: CCOC(=O)c1nc(Br)c(C)nc1N1CCC2(CC1)CO[C@@H](C)[C@@H]2N[S@@](=O)C(C)(C)C]

### EXAMPLES

The examples and preparations provided below further illustrate and exemplify the compounds and synthetic methods of the present disclosure. It is to be understood that the scope of the present disclosure is not limited in any way by the scope of the following examples.

The following abbreviations may be relevant for the application.

### Abbreviations

| | |
|---|---|
| Boc | tert-butyloxycarbonyl |
| Bu | butyl |
| DIBAL-H | diisobutylaluminum hydride |
| DIPEA | diisopropylethylamine |
| DCM | dichloromethane |
| DMF | N,N-dimethylformamide |
| DMAP | 4-dimethylaminopyridine |
| DMP | Dess-Martin Periodinane |
| DMSO | dimethylsulfoxide |
| EA or EtOAc | ethyl acetate |
| EP | expected product |
| eq. or equiv. | equivalents |
| Et | ethyl |
| EtOH | ethanol |
| FCC | Flash Column Chromatography |
| h or hr | hour(s) |
| HMDS | hexamethyldisilazane |
| HPLC | High-Performance Liquid Chromatography |
| LCMS | Liquid Chromatography Mass Spectrometry |
| LDA | lithium diisopropylamide |
| MTBE | methyl tert-butyl ether |
| MeOH | methanol |
| MeTHF | 2-methyltetrahydrofuran |
| OAc | acetate |
| PE | petroleum ether |
| NBS | N-bromosuccinimide |
| rt or RT | room temperature |
| TBAF | tetra-n-butylammonium fluoride |
| TBS | tert-butyldimethylsilyl |
| TBSCl | tert-butyldimethylsilyl chloride |
| TEMPO | 2,2,6,6-tetramethylpiperidin-1-yl)oxyl |
| THF | tetrahydrofuran |
| TLC | Thin Layer Chromatography |
| TMP | 2,2,6,6-tetramethylpiperidine |
| Tos | tosyl (toluenesulfonyl) |
| TsCl | tosyl chloride |
| vol. | volume(s) |
| Xantphos | 4,5-bis(diphenylphosphino)-9,9-dimethylxanthene |

### Example 1. Preparation of Ethyl (S)-6-bromo-5-methyl-3-(3-methyl-4-oxo-2-oxa-8-azaspiro[4.5]decan-8-yl)pyrazine-2-carboxylate (Compound (1))

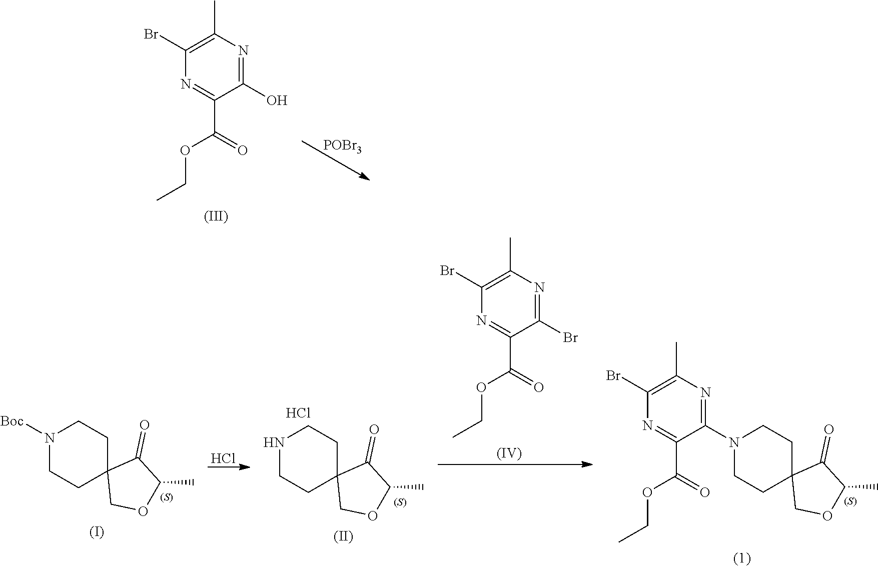

Compound (1) was prepared by the series of reactions shown in the scheme above. Compound (I) was deprotected by treatment with HCl to form Compound (II) or a salt thereof. Compound (IV) was prepared by reaction of Compound (III) with POBr₃. Next, Compound (II), or a salt thereof, was coupled to Compound (IV) to give Compound (1).

### Synthesis of Compound (I).

Route A. Compound (I) can be prepared according to the procedures described in U.S. Pat. No. 10,590,090.

Route B. Compound (I) can be prepared according to the reaction scheme shown below.

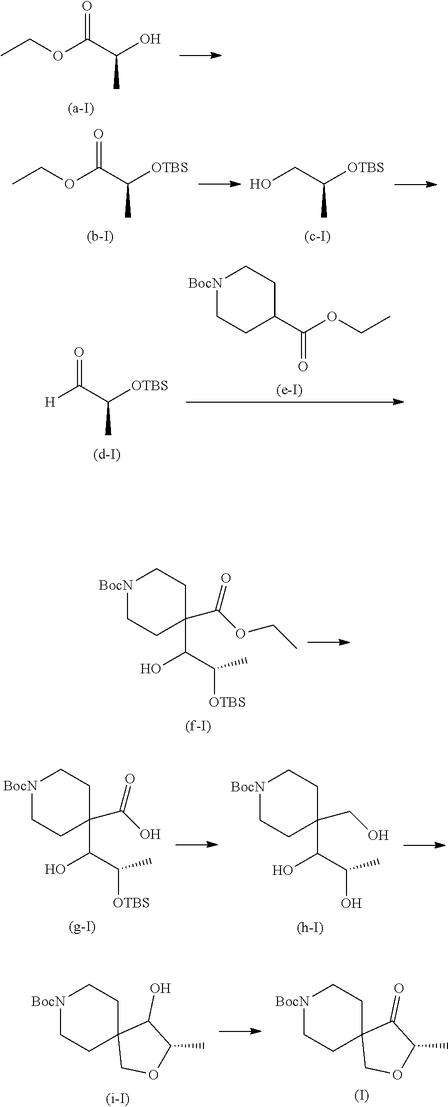

Step 1. Synthesis of (b-I).

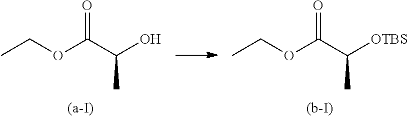

To a 20-L reactor was charged (−)-ethyl L-lactate (a-I) (1000 g, 8.47 mol, 1.0 equiv) and DCM (8000 mL, 8 vol). This reaction mixture was cooled to and maintained at 10° C. To this reaction mixture was charged imidazole (1152 g, 16.93 mol, 2 equiv). Once imidazole was completely dissolved, the mixture was cooled to −5° C. To this was charged TBSCl (1403 g, 9.31 mol, 1.1 equiv) in DCM (2000 mL, 2 vol). The reaction was then warmed to 22° C. and maintained at 22° C. for 1 hour during which time TLC monitoring (9/1 hexane/EtOAc, KMnO₄ as stain) showed reaction completion.

To the reaction mixture was charged water (4500 mL). The layers were separated and the organic layer was washed with water (3×4500 mL), 10% w/v citric acid in water (3×4500 mL), and water (1×4500 mL). The organic phase was concentrated under reduced pressure to afford ethyl (S)-2-((tert-butyldimethylsilyl)oxy)propanoate (b-I) as a colorless liquid (2016 g, >100% yield).

¹H NMR (300 MHz, DMSO-d₆) δ 4.34 (q, J=6.7 Hz, 1H), 4.10 (q, J=7.1 Hz, 2H), 1.29 (d, J=6.7 Hz, 3H), 1.20 (t, J=7.1 Hz, 3H), 0.87 (s, 9H), 0.06 (s, 3H), 0.05 (s, 3H).

Step 2. Synthesis of (c-I).

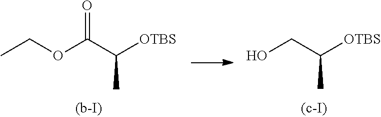

To a 20-L reactor was charged ethyl (S)-2-((tert-butyldimethylsilyl)oxy)propanoate (b-I) (1180 g, 5.08 mol, 1 equiv), methyl tert-butyl ether (MTBE) (11800 mL, 10 vol), and MeOH (226 mL, 5.59 mol, 1.1 equiv). This mixture was cooled to 0° C. To this was charged LiBH₄ (127.2 g, 5.84 mol, 1.15 equiv) in THF (2289 mL). The reaction was maintained at room temperature for 2 hours at which point TLC monitoring (9/1 hexane/EtOAc, KMnO₄ as stain) showed reaction completion.

To the reaction was charged ice water (6000 mL), during which time a white precipitate formed. The layers were separated, filtering off the solid with a Schott funnel, washing the solid with EtOAc. The combined organic phases were combined with a parallel reaction and concentrated to afford (S)-2-((tert-butyldimethylsilyl)oxy)propan-1-ol (c-I) as a slightly yellow liquid (1420 g, 97% total yield).

¹H NMR (300 MHz, DMSO-d₆) δ 4.54 (t, J=5.6 Hz, 1H), 3.74 (h, J=6.1 Hz, 1H), 3.30 (dt, J=11.0, 5.6 Hz, 1H), 3.15 (dt, J=10.5, 6.0 Hz, 1H), 1.05 (d, J=6.2 Hz, 3H), 0.86 (s, 9H), 0.04 (s, 6H).

Step 3. Synthesis of (d-I).

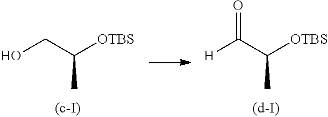

To a 6-L flask was charged (S)-2-((tert-butyldimethylsilyl)oxy)propan-1-ol (c-I) (300 g, 1.58 mol, 1 equiv), DCM (2100 mL, 7 vol), and TEMPO (12.3 g, 0.079 mol, 0.05 equiv). To this solution was charged a solution of KBr (93.8 g, 0.79 mol, 0.5 equiv) in water (281 mL, 3 vol). The resulting mixture was cooled to 3° C. To this was then charged a saturated aqueous solution of NaHCO₃ in ˜15% NaClO (2400 mL, 8 vol) over 1 hour. The reaction was maintained at 3° C. for 1 hour at which point TLC monitoring (9/1 hexane/EtOAc, p-anisaldehyde as stain) showed reaction completion.

The layers were separated and the aqueous layer extract with DCM (2×1500 mL). The combined organic layers were concentrated under reduced pressure to afford (S)-2-((tert-butyldimethylsilyl)oxy)propanal (d-I) as a reddish oil (317.5 g, >100% yield).

¹H NMR (300 MHz, DMSO-d₆) δ 9.54 (d, J=0.7 Hz, 1H), 4.25 (qd, J=6.9, 0.7 Hz, 1H), 1.21 (d, J=3.0 Hz, 3H), 0.89 (s, 9H), 0.08 (d, J=4.6 Hz, 6H).

Step 4. Synthesis of (f-I).

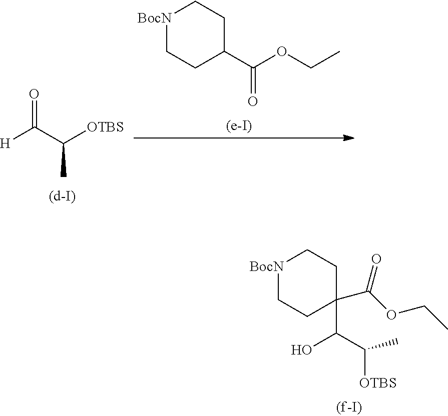 [SMILES: C[C@@H](C=O)O[Si](C)(C)C(C)(C)C ; CCOC(=O)C1CCN(C(=O)OC(C)(C)C)CC1 ; CCOC(=O)C1(C(O)[C@H](C)O[Si](C)(C)C(C)(C)C)CCN(C(=O)OC(C)(C)C)CC1]

To a 6-L flask was charged diisopropylamine (123 mL, 89 g, 0.88 mol, 1.65 equiv) and THE (1200 mL). This solution was cooled to −12° C. To this was charged n-BuLi (2.5M in hexane) (340 mL, 54 g, 0.85 mol, 1.6 equiv) and the resulting mixture was maintained for 1 hour and 15 minutes. To this was charged 1-(tert-butyl) 4-ethyl piperidine-1,4-dicarboxylate (e-I) (137 mL, 143 g, 0.56 mol, 1.05 equiv) and THE (290 mL) and the resulting mixture was maintained for 1 hour and 25 minutes at which time use test of an aliquot was performed and TLC monitoring (6/4 hexane/EtOAc, ninhydrin as stain) showed reaction completion.

The reaction mixture was warmed to 0° C. and to this was charged (S)-2-((tert-butyldimethylsilyl)oxy)propanal (d-I) (100 g, 0.53 mol, 1 equiv). The reaction was maintained at 0° C. or 1 hour at which point TLC monitoring (6/4 hexane/EtOAc, ninhydrin as stain) showed reaction completion.

To the reaction mixture was charged saturated aqueous NaHCO₃ and water (1:4, 700 mL), maintaining a temperature of ≤2° C. To this was then charged EtOAc (1000 mL). The layers were separated and the aqueous layer was extracted with EtOAc (2×500 mL). The combined organic layers were dried over anhydrous Na₂SO₄, filtered, combined with a parallel reaction, and concentrated to afford 1-(tert-butyl) 4-ethyl 4-((2S)-2-((tert-butyldimethylsilyl)oxy)-1-hydroxypropyl)piperidine-1,4-dicarboxylate (f-1) as an orange oil (532.8 g, 97% total yield).

¹H NMR (300 MHz, DMSO-d₆) δ 5.16 (d, J=6.9 Hz, 1H), 4.13-4.03 (m, 2H), 3.93-3.77 (m, 2H), 3.70 (t, J=5.9 Hz, 1H), 3.41 (t, J=6.4 Hz, 1H), 2.83 (s, 1H), 1.88-1.49 (m, 5H), 1.42-1.36 (m, 9H), 1.12 (d, J=5.9 Hz, 3H), 0.85 (d, J=1.3 Hz, 9H), 0.03 (d, J=4.3 Hz, 6H).

Step 5. Synthesis of (g-I).

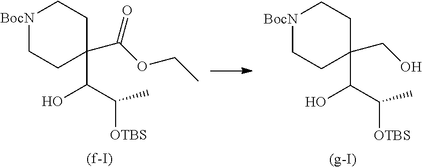 [SMILES: CCOC(=O)C1(C(O)[C@H](C)O[Si](C)(C)C(C)(C)C)CCN(C(=O)OC(C)(C)C)CC1 ; C[C@H](O[Si](C)(C)C(C)(C)C)C(O)C1(CO)CCN(C(=O)OC(C)(C)C)CC1]

To a 6-L flask at room temperature was charged 1-(tert-butyl) 4-ethyl 4-((2S)-2-((tert-butyldimethylsilyl)oxy)-1-hydroxypropyl)piperidine-1,4-dicarboxylate (f-I) (530 g, 1.2 mol, 1 equiv) and THE (4200 mL). To this was charged LiBH₄ (38.9 g, 1.8 mol, 1.5 equiv) in portions, temperature raising from 21° C. to 30° C. The reaction was maintained at room temperature overnight at which point TLC monitoring (6/4 hexane/EtOAc, ninhydrine as stain) showed reaction completion.

The reaction was cooled to 0° C. and to this was charged saturated aqueous NaHCO₃ and water (1:2; 1500 mL) and then EtOAc (500 mL). The layers were separated. The organic layer was filtered through a Schott funnel to remove precipitation and then washed with brine (3×2000 mL), dried over anhydrous Na₂SO₄, filtered, and concentrated to give tert-butyl 4-((2S)-2-((tert-butyldimethylsilyl)oxy)-1-hydroxypropyl)-4-(hydroxymethyl)piperidine-1-carboxylate (g-I) as an orange oil (480 g, 100% yield).

¹H NMR (300 MHz, DMSO-d₆) δ 4.55 (t, J=4.8 Hz, 1H), 4.44 (dd, J=5.8, 3.0 Hz, 1H), 4.04 (dd, J=6.5, 4.3 Hz, 1H), 3.50 (dd, J=11.1, 5.7 Hz, 4H), 3.27-3.20 (m, 1H), 3.03 (s, 2H), 1.67-1.46 (m, 2H), 1.39 (s, 9H), 1.18 (td, J=6.4, 5.7, 2.7 Hz, 1H), 1.12 (d, J=6.0 Hz, 3H), 0.85 (d, J=3.4 Hz, 9H), 0.05 (d, J=2.6 Hz, 6H).

Step 6. Synthesis of (h-I).

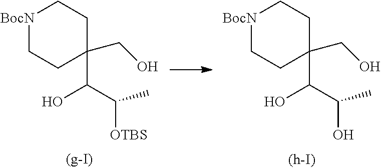 [SMILES: C[C@H](O[Si](C)(C)C(C)(C)C)C(O)C1(CO)CCN(C(=O)OC(C)(C)C)CC1 ; C[C@H](O)C(O)C1(CO)CCN(C(=O)OC(C)(C)C)CC1]

To a 6-L flask was charged tert-butyl 4-((2S)-2-((tert-butyldimethylsilyl)oxy)-1-hydroxypropyl)-4-(hydroxymethyl)piperidine-1-carboxylate (g-I) (245 g, 0.61 mol, 1 equiv) and THE (1960 mL). To this solution was charged a solution of TBAF 3H₂O (287 g, 0.91 mol, 1.5 equiv) in THE (500 mL). The resulting mixture was maintained at room temperature for 45 minutes at which point TLC monitoring (7/3 hexane/EtOAc, ninhydrin as stain) showed reaction completion.

To the reaction was charged saturated aqueous NaHCO₃ and water (1:2, 1000 mL) and EtOAc (500 mL). The layers were separated. The aqueous phase was extracted with EtOAc (300 mL). The combined organic layers were washed with brine (2×500 mL), dried over anhydrous Na₂SO₄, filtered, and concentrated (combining with the filtrate of a duplicate parallel batch) to afford crude tert-butyl 4-((2S)-1,2-dihydroxypropyl)-4-(hydroxymethyl)piperidine-1-carboxylate (h-I) as a dense orange oil (810 g).

The crude was purified by silica gel column chromatography (crude was absorbed on SiO₂ (1 g SiO₂/1 g crude), column was prepared: 10 g SiO₂/1 g crude, eluent: hexane→hexane/EtOAc (9/1)→EtOAc). Isolation afforded tert-butyl 4-((2S)-1,2-dihydroxypropyl)-4-(hydroxymethyl)piperidine-1-carboxylate (h-I) as a dense yellow oil (195.6 g, 56% yield).

¹H NMR (300 MHz, DMSO-d₆) δ 4.73 (t, J=5.2 Hz, 1H), 4.63 (d, J=5.2 Hz, 1H), 4.51 (d, J=6.8 Hz, 1H), 3.71 (h, J=6.1 Hz, 1H), 3.50 (dq, J=11.3, 5.5 Hz, 4H), 3.19-2.95 (m, 4H), 1.61 (dddd, J=24.0, 14.3, 10.0, 4.4 Hz, 2H), 1.39 (s, 9H), 1.11 (d, J=6.1 Hz, 3H).

Step 7. Synthesis of (i-I).

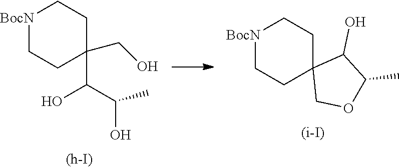 [SMILES: C[C@H](O)C(O)C1(CO)CCN(C(=O)OC(C)(C)C)CC1 ; C[C@@H]1OCC2(CCN(C(=O)OC(C)(C)C)CC2)C1O]

To a 6-L flask was charged NaH (60% w/w suspension in mineral oil, 46.8 g gross, 28.8 g net, 1.17 mol, 3.5 equiv) and THE (1200 mL). This was cooled to −15° C. To this was charged a solution of tert-butyl 4-((2S)-1,2-dihydroxypropyl)-4-(hydroxymethyl)piperidine-1-carboxylate (h-I) (96.7 g, 0.33 mol, 1 equiv) in THE (440 mL) followed by a solution of pTsCl (64 g, 0.33 mol, 1 equiv) in THE (540 mL). The reaction was maintained at −10° C. for 35 minutes at which point TLC monitoring (7/3 EtOAc/hexane, ninhydrin as stain) showed reaction completion.

The reaction mixture was cooled to −25° C. To this was charged saturated aqueous of NH₄Cl (550 mL) and EtOAc (500 mL). The layers were separated. The aqueous layers were extracted with EtOAc (2×500 mL). The combined organic layers were dried over anhydrous Na₂SO₄, filtered, concentrated (combing with the filtrate of a duplicate parallel batch) to afford crude tert-butyl (3S)-4-hydroxy-3-methyl-2-oxa-8-azaspiro[4.5]decane-8-carboxylate (i-I) (243.7 g).

The crude was suspended in Et20 (300 mL) and the mixture was stirred for 10 minutes. The resulting solid was filtered, washing the cake with Et20 (50 mL). The combined filtrates were concentrated to afford tert-butyl (3S)-4-hydroxy-3-methyl-2-oxa-8-azaspiro[4.5]decane-8-carboxylate (i-I) as a yellowish oil (210.2 g, >100% yield).

¹H NMR (300 MHz, DMSO-d₆) δ 5.04 (d, J=5.4 Hz, 1H), 3.63 (d, J=8.9 Hz, 1H), 3.57-3.51 (m, 1H), 3.26-3.20 (m, 1H), 3.03-2.86 (m, 2H), 1.63-1.44 (m, 1H), 1.39 (d, J=2.0 Hz, 9H), 1.27 (d, J=18.4 Hz, 2H), 1.21-1.15 (m, 5H).

Step 8. Synthesis of Compound (I).

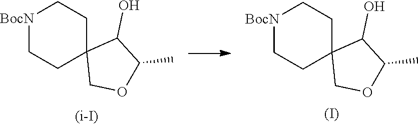

To a 6-L flask was charged tert-butyl (3S)-4-hydroxy-3-methyl-2-oxa-8-azaspiro[4.5]decane-8-carboxylate (i-I) (177 g, 0.65 mol, 1 equiv) and DCM (1700 mL). This was cooled to −12° C. To this was charged Dess-Martin periodinane (553 g, 1.3 mol, 2 equiv). The reaction was maintained at −12° C. for 4 hours at which point TLC monitoring (7/3 EtOAc/hexane, ninhydrin as stain) showed incomplete reaction. To the reaction was charged additional Dess-Martin periodinane (55.4 g, 0.13 mol, 0.2 equiv). The reaction was maintained at −12° C. overnight at which point TLC monitoring showed incomplete reaction. To the reaction was charged additional Dess-Martin periodinane (83 g, 0.19 mol, 0.3 equiv) followed by additional Dess-Martin periodinane (138.4 g, 0.33 mol, 0.5 equiv) after 30 minutes. The reaction was maintained for 3 hours.

To the reaction was charged saturated aqueous NaHCO₃ (2000 mL) and DCM (500 mL). The layers were separated. The aqueous layer was extracted with DCM (2×800 mL). The combined organic layers were washed with water (3×1000 mL), dried over anhydrous Na₂SO₄, filtered, and concentrated to afford crude tert-butyl (S)-3-methyl-4-oxo-2-oxa-8-azaspiro[4.5]decane-8-carboxylate (I) as an oil (249.7 g).

The crude was purified by silica gel column chromatography (crude was absorbed on SiO₂ (1 g SiO₂/1 g crude), column was prepared with 10 g SiO₂/1 g crude, eluent: hexane→95/5 hexane/EtOAc). Isolation afforded tert-butyl (S)-3-methyl-4-oxo-2-oxa-8-azaspiro[4.5]decane-8-carboxylate (I) as an oil (90.8 g, 52% yield).

¹H NMR (300 MHz, DMSO-d₆) δ 4.22 (d, J=9.5 Hz, 1H), 4.03-3.92 (m, 1H), 3.83 (dd, J=9.6, 1.1 Hz, 1H), 3.72 (tt, J=13.7, 4.6 Hz, 2H), 3.08 (t, J=12.0 Hz, 1H), 2.94 (d, J=22.4 Hz, 2H), 1.57-1.46 (m, 3H), 1.40 (d, J=3.5 Hz, 9H), 1.19 (d, J=6.9 Hz, 3H).

Route C. Compound (I) can be prepared according to the reaction scheme shown below. This synthetic route is similar to Route B above but requires fewer intermediates.

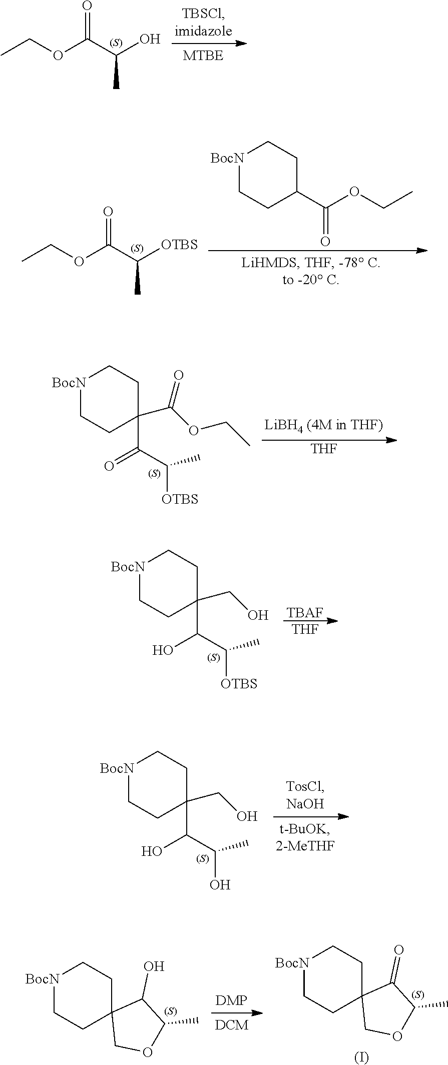

### Synthesis of Compound (II).

Compound (II) was prepared by reacting Compound (I) with HCl.

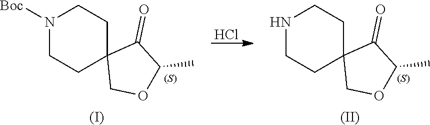

Synthesis A. Compound (I) (10 g, 1 equiv.) was treated with HCl (9.28 mL, 36%, 3 equiv.) in acetone (50 mL) at 20-30° C. for 4 hours. Additional acetone (90 mL) was added and the reaction mixture was stirred overnight. The solid precipitate was collected by filtration, washed with acetone (20 mL), and dried at 30-40° C. under vacuum. Compound (II) was isolated in good yield (5.2 g, 83%) and in high purity (HPLC purity: >99%; chiral purity: >99%).

Synthesis B. Compound (I) (500 mg) was treated with HCl (4.5 equiv.) in dioxane solution (4 M) at 0° C. to rt. The solid precipitate was collected by filtration and washed with dioxane. Compound (II) was isolated in 75% yield, in high purity (HPLC purity >99%; chiral purity: >99%).

### Synthesis of Compound (III).

Route A. Compound (III) can be prepared according to the procedures described in U.S. Pat. No. 10,590,090 as shown in the scheme below.

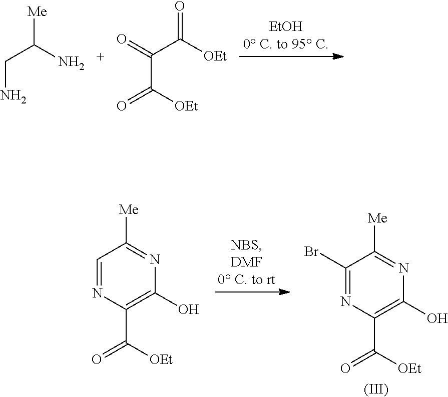

Route B. Compound (III) was prepared according to the scheme below. In this reaction, hydrated ketomalonate replaces the ketomalonate of Route A above.

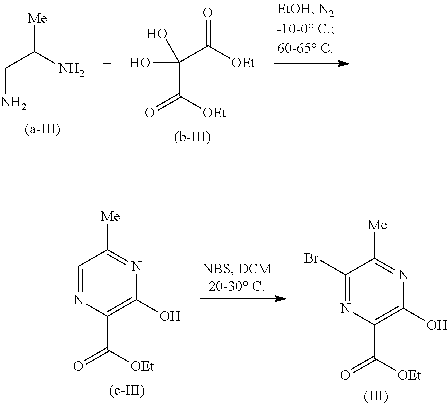

Step 1. Synthesis of (c-III).

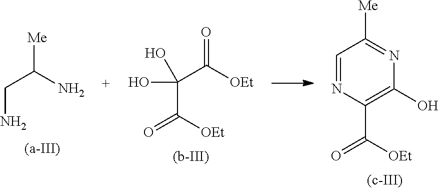 [SMILES: CC(N)CN ; CCOC(=O)C(O)(O)C(=O)OCC ; CCOC(=O)c1ncc(C)nc1O]

To a 1500-L reactor was charged EtOH (810 kg, 5 vol). The reactor was evacuated and backfilled with nitrogen atmosphere two times. To the reactor was charged propane-1,2-diamine (a-III) (68.4 kg, 922.8 mol, 1.05 equiv). The resulting mixture was cooled to −8 to −15° C. To the reactor was then charged diethyl 2,2-dihydroxymalonate (b-III) (168.9 kg, 878.9 mol, 1.0 equiv) in fifteen equal portions over 4 hours, maintaining a temperature of −10 to 0° C. The reaction mixture was maintained at −10 to 0° C. for 2 hours during which time a white solid precipitated and at which point GC monitoring showed first reaction completion. The reaction mixture was warmed to 60-65° C. during which time a clear solution formed. The reaction mixture was maintained at 60-65° C. for 15 hours at which point HPLC monitoring showed second reaction completion.

The reaction was cooled to 25-35° C. and then distilled to ˜1.2 vol, maintaining temperature below 45° C. The concentrate was cooled to 0-5° C. over 1 hour and then maintained at 0-5° C. for 1 hour. To this mixture was charged methyl tert-butyl ether (MTBE) (63.0 kg, 0.5 vol) over 1 hour, maintaining a temperature of 0-5° C. The resulting mixture was maintained at 0-5° C. for 1 hour and then filtered, washing the cake with EtOH/MTBE (2×1:1 (v/v), 68.0 kg).

The cake was dried under reduced pressure at 40-45° C. for 13 hours to afford ethyl 3-hydroxy-5-methylpyrazine-2-carboxylate (c-III) as a brick-red solid (32.9 kg, 99.1% a/a HPLC purity, 96.9% w/w qNMR assay, 21% assay-corrected yield).

¹H NMR (400 MHz, DMSO-d₆) δ 12.74 (br s, 1H), 7.36 (s, 1H), 4.26 (q, J=7 Hz, 2H), 2.24 (s, 3H), 1.26 (t, J=6 Hz, 3H).

Step 2. Synthesis of Compound (III).

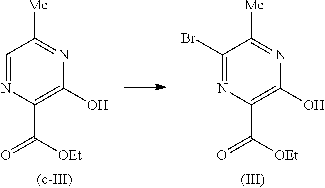

To a 1000-L reactor was charged ethyl 3-hydroxy-5-methylpyrazine-2-carboxylate (c-III) (32.9 kg, 96.9% w/w assay, 175.0 mol, 1.0 equiv) and DCM (450 kg, 10 vol). The reactor was evacuated and backfilled with nitrogen atmosphere two times. To the reactor was charged NBS (32.1 kg, 180.4 mol, 1.03 equiv) in five equal portions over 2.5 hours, maintaining a temperature of 20-30° C. The reaction mixture was maintained at 20-30° C. for 1 hour at which point was charged more NBS (400 g, 2.2 mol, 0.01 equiv), maintaining a temperature of 20-30° C. The reaction mixture was maintained at 20-30° C. for 0.5 hours as which point HPLC monitoring showed reaction completion.

To the reaction was charged 5% w/w aqueous NaHSO₃ (160 kg, 5 vol). The layers were separated. The organic layer was washed with water (160 kg, 5 vol), brine (160 kg, 5 vol), and then filtered. The filtrate was concentrated to ˜2 vol (˜70 L). To the concentrate was added MTBE (45 kg, 2.0 vol). This was concentrated to ˜2 vol (˜70 L). To the concentrate was added n-heptane (112 kg, 5 vol) over 1.5 hours, maintaining a temperature of 10-20° C. The resulting suspension was maintained at 10-20° C. for 1 hour and then filtered. The cake was dried at 40-45° C. for 5 hours to afford ethyl 6-bromo-3-hydroxy-5-methylpyrazine-2-carboxylate (III) as a light-orange solid (37.8 kg, 99.6% a/a HPLC purity, 81% assay-corrected yield).

¹H NMR (400 MHz, CDCl₃) δ 11.24 (br s, 1H), 4.51 (q, J=7 Hz, 2H), 2.66 (s, 3H), 1.44 (t, J=6 Hz, 3H).

MS (ESI+): calculated, 260.00; found, 260.7.

### Synthesis of Compound (IV).

Compound (IV) was prepared according to the scheme below.

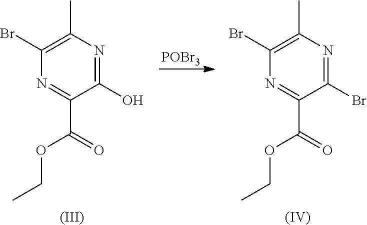

Synthesis A. Compound (III) (60 g, 1 equiv.) was treated with POBr₃ (85.7 g, 1.3 equiv.) and DMF (2.7 g, 0.16 equiv.) in DCM (450 mL) at 20-30° C. for 90 h. To the reaction mixture was added K₂CO₃ (1320 g, 10% solution) and the organic layer was washed with water. The solvent was removed under reduced pressure to yield Compound (IV) (75.6 g, 94% yield).

Synthesis B. To a reactor was charged DCM (458 kg), POBr₃ (58.0 kg, 1.3 equiv), and DMF (2.2. kg, 0.2 equiv). The mixture was maintained at 20-30° C. for 1 hour. To the reactor was then charged ethyl 6-bromo-3-hydroxy-5-methylpyrazine-2-carboxylate (III) (40.0 kg, 1 equiv) and additional DCM (22 kg). The reaction was heated to 30-40° C. and maintained at that temperature for 60 hours at which point UPLC monitoring showed reaction completion.

The reaction was then cooled to 20-30° C. To the reactor was charged 10% w/w aq. K₂CO₃ (851 kg), maintaining a temperature below 30° C. The layers were separated and the aqueous layer extracted with DCM (280 kg). The combined organic layers were washed with water (2×220 kg) and then concentrated to ˜4 vol under reduced pressure at ≤35° C. to afford ethyl 3,6-dibromo-5-methylpyrazine-2-carboxylate (IV) as a DCM solution (160.4 kg, 98.3% a/a UPLC purity, 30.0% w/w assay, 97% assay-corrected yield).

### Synthesis of Compound (1).

Compound (1) was prepared according to the scheme below.

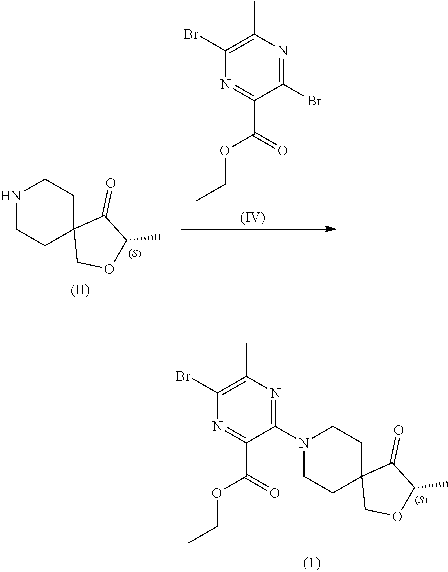

Compound (II) (200 g, 1 equiv.) was treated with Compound (IV) (135 g, 1.05 equiv.) and triethylamine (156.2 g, 2.5 equiv.) in acetone (1400 mL). After stirring for 20 h. at 20-30° C., acetic acid (18.5 g, 0.5 equiv.) was added at 15-25° C. Next, water (1400 mL) was slowly added and the reaction mixture was stirred for 1.5 h. at 20-30° C. The resulting solid was isolated by filtration and washed with water/acetone (1:1 v/v) to afford Compound (1) (236.5 g, 91.3% yield) with chiral purity >99%.

### Example 2. Preparation of Ethyl 6-bromo-3-[(3S)-4-[(S)-tert-butylsulfinyl]imino-3-methyl-2-oxa-8-azaspiro[4.5]decan-8-yl]-5-methyl-pyrazine-2-carboxylate (Sulfinyl Imine Compound (2b))

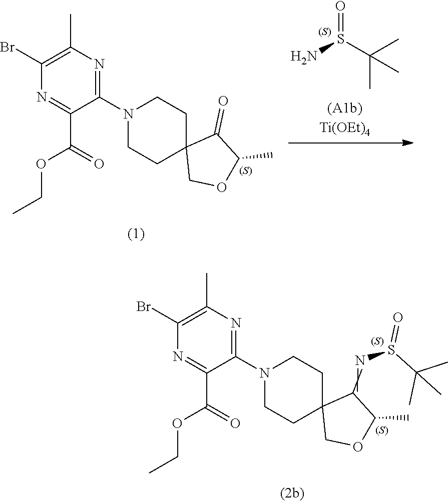

As described in detail below and in Example 3, the sulfinyl imine Compounds (2b) and (2a) were prepared from Compound (1) and (S)-sulfinamide (A1b) or (R)-sulfinamide (A1a), respectively. In each of the methods studied, the expected product (“EP” or “Compound (2a)” or “Compound (2b)”) was formed in addition to the corresponding isopropyl ester. As shown in Example 6, the presence of the isopropyl ester did not affect the synthesis of subsequent molecules as both esters (ethyl and isopropyl) were ultimately reduced to the corresponding alcohol. As such, it is not necessary to purify the product containing Compound (2a) or Compound (2b) to remove the corresponding isopropyl ester.

Methods 1-3 (small scale tests). Three small scale syntheses were conducted to determine the optimal amount of titanium reagent to be used. As summarized below in Table 1, no major drawbacks appeared in terms of conversion or impurity profile, and 2 equivalents of Ti(OEt)₄ was selected for scale-up.

TABLE 1

| Method No. | Scale | Conditions | Results |
|---|---|---|---|
| 1 | 500 mg | Compound (1): 1 eq. | 437.4 mg isolated (mixture |
| | | MeTHF: 10 vol. | of EP and isopropyl ester) |
| | | Ti(OEt)₄: 4 eq. | |
| | | S-sulfinamide (A1b): 1.1 eq | LCMS: |
| | | reflux | EP - 2 peaks 81.4% + 2.75% |
| | | 2 hr: 59% conversion | Isopropyl ester - 12.81% |
| | | 21 hr: 90.6% conversion | |
| | | 24 hr: 91.6% conversion | |
| 2 | 500 mg | Compound (1): 1 eq. | 464 mg isolated (mixture of |
| | | MeTHF: 10 vol. | EP and isopropyl ester) |
| | | Ti(OEt)₄: 3 eq. | |
| | | S-sulfinamide (A1b): 1.1 eq | LCMS: |
| | | reflux | EP - 2 peaks 83.2% + 2.53% |
| | | | Isopropyl ester - 12.25% |
| | | 2 hr: 56.3% conversion | |
| | | 21 hr: 90.2% conversion | |
| | | 24 hr: 93.6% conversion | |
| 3 | 500 mg | Compound (1): 1 eq. | 472 mg isolated (mixture of |
| | | MeTHF: 10 vol. | EP and isopropyl ester) |
| | | Ti(OEt)₄: 2 eq. | |
| | | S-sulfinamide (A1b): 1.1 eq | LCMS: |
| | | reflux | EP - 2 peaks 84.12% + 2.53% |
| | | | Isopropyl ester -11.215% |
| | | 2 hr: 52.5% conversion | |
| | | 21 hr: 86.8% conversion | |
| | | 24 hr: 89.4% conversion | |

Method 4 (large scale). In a 10 mL vial at room temperature was introduced, under N₂, ethyl (S)-6-bromo-5-methyl-3-(3-methyl-4-oxo-2-oxa-8-azaspiro[4.5]decan-8-yl)pyrazine-2-carboxylate (Compound (1)) (500 mg, 1.21 mmol) and anhydrous 2-methyltetrahydrofuran (2 mL). To the yellow solution was added dropwise, over 2 min, a solution of Ti(OEt)₄ (96%, 530 L, 2.43 mmol) in anhydrous 2-methyltetrahydrofuran (1.5 mL). To this solution was then added dropwise, over 2 min, a solution of 2-methylpropane-2-sulfinamide (S) (A1b) (162 mg, 1.33 mmol) in anhydrous 2-methyltetrahydrofuran (1.5 mL). The resulting yellow solution was then heated up to 80° C. for 24 h until conversion reached 89%. The reaction mixture was then cooled down to 0-5° C. using an ice bath.

To the cooled reaction mixture was added an aqueous citric acid solution (2.5 mL, 1 M, 5 vol.). To the thick suspension was added 2-methyltetrahydrofuran (2.5 mL, 5 vol.). The reaction mixture turned to a thin suspension that was filtered. The solid was washed 4 times with 2-methyltetrahydrofuran (2.5 mL, 4×5 vol.) to recover all EP. The layers were separated, and the organic layer was washed twice with an aqueous citric acid solution (1 M, 5 mL, 2×10 vol.) and four times with water (5 mL, 4×10 vol.). Alternatively, the work-up procedure was performed using water in place of the aqueous citric acid solution as described in Example 3.

The organic layer was concentrated to dryness to afford crude material (655 mg), which was purified by flash chromatography to obtain the expected product (“EP” or Compound (2b)) as a yellow oil in a mixture of ethyl and isopropyl ester in a ratio 85/15 (472 mg, 63% yield (ethyl ester)).

The product was analyzed using LCMS (UPLC Column Acquity UPLC CSH C18, 2.1×50 mm, 1.7 μm; eluant A=H₂O+0.02% HCOOH; eluent B=CH₃CN+0.02% HCOOH; oven temp=55° C.; gradient: t0 2% B, t4.5 min 98% B, t5 min 2% B; flow rate=1 mL/min; electrospray ionization mode—capillary, 3 kV sample cone 15/30V). LCMS analysis showed peaks at 514 (ethyl ester) and 528 (isopropyl ester).

### Example 3. Preparation of Ethyl 6-bromo-3-[(3S)-4-[(R)-tert-butylsulfinyl]imino-3-methyl-2-oxa-8-azaspiro[4.5]decan-8-yl]-5-methyl-pyrazine-2-carboxylate (Sulfinyl Imine Compound (2a))

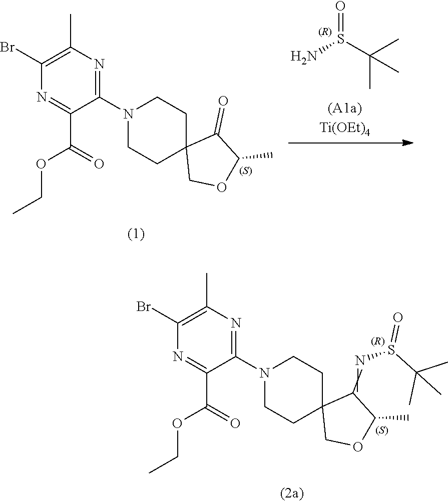

Small scale synthesis. The sulfinyl imine Compound (2a) was prepared from the (R)-sulfinamide (A1a) following the same protocol as used for the (S)-sulfinamide in Example 2. The retention times of the (R) (Compound (2a)) and (S) (Compound (2b)) diastereoisomers were similar, with the (R)-sulfinamide analog (Compound (2a)) being slightly less polar.

Large scale synthesis. In a 250 mL round-bottom flask at room temperature was introduced, under N₂, ethyl (S)-6-bromo-5-methyl-3-(3-methyl-4-oxo-2-oxa-8-azaspiro[4.5]decan-8-yl)pyrazine-2-carboxylate (Compound (1)) (5 g, 12.1 mmol) and anhydrous 2-methyltetrahydrofuran (25 mL). To this yellow solution was added dropwise, over 15 min, a solution of 2-methylpropane-2-sulfinamide (R) (A1a) (2.94 g, 24.3 mmol, 2 eq.) in 2-anhydrous methyltetrahydrofuran (12.5 mL). To the yellow solution was added dropwise, over 20 min, a solution of Ti(OEt)₄ (99.99%, 4.6 mL, 21.8 mmol, 1.8 eq.) in anhydrous 2-methyltetrahydrofuran (12.5 mL). The resulting clear yellow solution was then heated at 80° C. for 15 h. and complete conversion was observed. The reaction mixture was then cooled down to room temperature. To the reaction mixture was added an aqueous citric acid solution (30 mL, 1 M). The reaction mixture turned to a thin suspension that was filtered on cardboard. The solid was washed 4 times with 2-methyltetrahydrofuran (20 mL, 4×4 vol.). The layers were separated, and the organic layer was washed twice with an aqueous citric acid solution (30 mL, 1 M, 2×6 vol.) and four times with water (30 mL, 4×6 vol.). The organic layer was concentrated to dryness to afford crude product containing Compound (2a) (6.24 g) and about 10-20% of the corresponding isopropyl ester. Alternatively, the work-up procedure was performed using water in place of the aqueous citric acid solution. Briefly, once the reaction was complete, the mixture was cooled to 20-25° C. and water (8 equiv. relative to Ti(OEt)₄) was added to the orange solution. The suspension was filtered over a pad of Celite® and the cake was washed with MeTHF (4*5 vol). The yellow filtrate was kept as a solution of MeTHF containing Compound (2a) and the corresponding isopropyl ester.

The crude product was analyzed using the same LCMS conditions described in Example 2 for Compound (2b). LCMS analysis showed a peak at 514 (ethyl ester) and an impurity peak at 528 (isopropyl ester). ¹H NMR (DMSO-d₆, 500 MHz): δ (ppm) 4.88 (q, J=6.4 Hz, 1H), 4.31 (q, J=7.1 Hz, 2H), 3.98 (q, J=9.5 Hz, 2H), 3.68-3.90 (m, 2H), 3.02-3.21 (m, 2H), 2.48 (s, 3H), 1.56-1.87 (m, 4H), 1.42 (d, J=6.8 Hz, 3H), 1.27-1.33 (m, 3H), 1.17 (s, 9H).

### Example 4. Preparation of (R/S)—N-[(3S,4S)-8-[5-bromo-3-(hydroxymethyl)-6-methyl-pyrazin-2-yl]-3-methyl-2-oxa-8-azaspiro[4.5]decan-4-yl]-2-methyl-propane-2-sulfinamide (Compound (3a) and Compound (3b′))

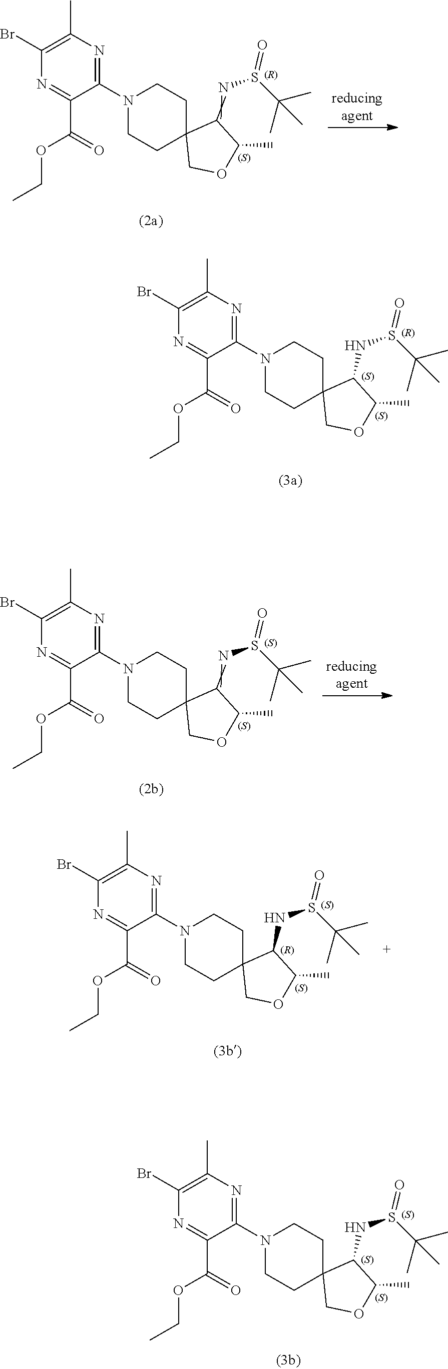

Methods 1-8—reducing the (R)-sulfinyl imine (Compound (2a)). The reduction of the (R)-sulfinyl imine (Compound (2a)) was examined using four different reducing agents under varying conditions (Table 2). Methods 1-4 varied the reducing agent, and methods 5-8 examined the effect of temperature on reductions with DIBAL-H.

TABLE 2

| Method No. | Scale | Conditions | Results |
|---|---|---|---|
| 1 | 100 mg | 1 eq. R-sulfinyl imine | Expected MW observed |
| | | MeTHF (10 vol.) | but no EP (reduction |
| | | L-selectride (1M, | occurred but product does |
| | | 1.5 eq) −78° C. | not have desired |
| | | 2 hr: 75% conversion | stereochemistry; obtained |
| | | | 3S,4R isomer) |
| 2 | 100 mg | 1 eq. R-sulfinyl imine | Degradation after |
| | | MeTHF (10 vol.) | additional 1.5 eq. LiBHEt₃ |
| | | LiBHEt₃ (1M, | |
| | | 2*1.5 eq) −78° C. | |
| | | 2 hr: MW = 517 and | |
| | | degradation products | |
| 3 | 100 mg | 1 eq. R-sulfinyl imine | MW observed after |
| | | MeTHF (10 vol.) | warming at 20-25° C., no |
| | | NaBH₄ | complete conversion |
| | | (2*4 eq) −78° C. | (84%), no degradation after |
| | | 2 hr: no reaction at −78° C. | treatment |
| 4 | 172 mg | 1 eq. R-sulfinyl imine | 141 mg isolated (88% |
| | | MeTHF (10 vol.) | LCMS) after FCC; mixture |
| | | DIBAL-H (1M, | between EP (Compound |
| | | 1.5 eq) −78° C. | (3a)) and isopropyl ester, |
| | | 2 hr: complete conversion | yield 80% |
| 5 | 100 mg | 1 eq. R-sulfinyl imine | Clean profile |
| | | MeTHF (10 vol.) | |
| | | DIBAL-H (1M, | |
| | | 1.5 eq) −50° C. | |
| | | 1.5 hr: complete conversion | |
| 6 | 100 mg | 1 eq. R-sulfinyl imine | Clean profile |
| | | MeTHF (10 vol.) | |
| | | DIBAL-H (1M, | |
| | | 1.5 eq) −20° C. | |
| | | 1.5 hr: complete conversion | |
| 7 | 100 mg | 1 eq. R-sulfinyl imine | Clean profile |
| | | MeTHF (10 vol.) | |
| | | DIBAL-H (1M, | |
| | | 1.5 eq) 0° C. | |
| | | 1.5 hr: complete conversion | |
| 8 | 100 mg | 1 eq. R-sulfinyl imine | Complete conversion but |
| | | MeTHF (10 vol) | some impurities observed |
| | | DIBAL-H (1M, 1.5 eq) | |
| | | Room temperature | |
| | | 1.5 hr: complete conversion | |

Methods 9-13—reducing the (S)-sulfinyl imine (Compound (2b)). The reduction of the (S)-sulfinyl imine (Compound (2b)) was examined using four different reducing agents (Table 3).

TABLE 3

| Method No. | Scale | Conditions | Results |
|---|---|---|---|
| 9 | 200 mg | 1 eq. S-sulfinyl imine | MW observed but not same |
| | | MeTHF (10 vol.) | retention time; reduction of |
| | | L-selectride (1M, | pyrimidine suspected |
| | | 2*1.5 eq), −78° C. | because of different rt; after |
| | | 2 hr: 50% conversion | purification, complete |
| | | | degradation to MW = 353 |
| | | | without Br, unidentified |
| | | | compound |
| 10 | 200 mg | 1 eq. S-sulfinyl imine | Several peaks with expected |
| | | MeTHF (10 vol.) | MW. Degradation also |
| | | LiBHEt₃ (1M, | observed. Attempt to purify |
| | | 2*1.5 eq), −78° C., 2 hr | by FCC not successful |
| 11 | 200 mg | 1 eq. S-sulfinyl imine | No reaction at −78° C. |
| | | MeTHF (10 vol.) | Reduction seems to occur at |
| | | NaBH₄ | higher temp. After 5 days at |
| | | (2*4 eq) −78° C., 2 hr | rt, reduction of ester moiety |
| 13 | 200 mg | 1 eq. S-sulfinyl imine | 112.7 mg isolated after FCC; |
| | | MeTHF (10 vol.) | mixture between EP |
| | | DIBAL-H (1M, | (Compound (3b′)) and EP- |
| | | 2*1.5 eq) −78° C. | isopropyl; corrected yield = |
| | | 2 hr: 32.9% conversion | 58% |

Compound (3b), EP (ethyl ester), 3S(Me), 4S(NH): Rt1=3.31 min (9.1%); Compound (3b′), EP (ethyl ester), 3S(Me), 4R(NH): Rt2=3.40 min (66.2%); Compound (3b′), isopropyl ester, 3S(Me), 4R(NH): Rt3=3.59 min (9.1%); Compound (3b) isopropyl ester, 3S(Me), 4S(NH): Rt6=3.5 min (1.1%).

Method 14—reducing the (R)-sulfinyl imine with DIBAL-H (scale-up). In a 3-neck round bottom flask at room temperature was introduced, under N₂ atmosphere, ethyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (Compound (2a)) (1.0091 g, 1.4 mmol) and MeTHF (10 mL). The reaction mixture was cooled down to −78° C. and then DIBAL-H (2.1 mL, 1 M in THF) was added. The reaction mixture was stirred at −78° C. for 1 h and quenched with Rochelle salt. The organic layer was separated and concentrated to dryness before flash chromatography purification (CH₂Cl₂/acetone). Compound (3a) was isolated as a yellow oil (571 mg, 79%). LCMS analysis showed a peak at 516. ¹H NMR (500 MHz, DMSO-d₆, 300K) δ ppm 5.10 (d, J=10.8 Hz, 1H), 4.32 (q, J=7.1 Hz, 2H), 4.13 (t, J=6.2 Hz, 1H), 3.82 (d, J=8.8 Hz, 1H), 3.73 (br d, J=13.7 Hz, 1H), 3.60 (br d, J=13.7 Hz, 1H), 3.50 (d, J=8.8 Hz, 1H), 3.41 (dd, J=10.8, 6.1 Hz, 1H), 3.03-3.21 (m, 2H), 2.48 (s, 3H), 1.69-1.85 (m, 2H), 1.49-1.64 (m, 2H), 1.31 (t, J=7.2 Hz, 3H), 1.15 (s, 9H), 1.09 (d, J=6.4 Hz, 3H).

### Example 5. Preparation of Amino Compound (4)

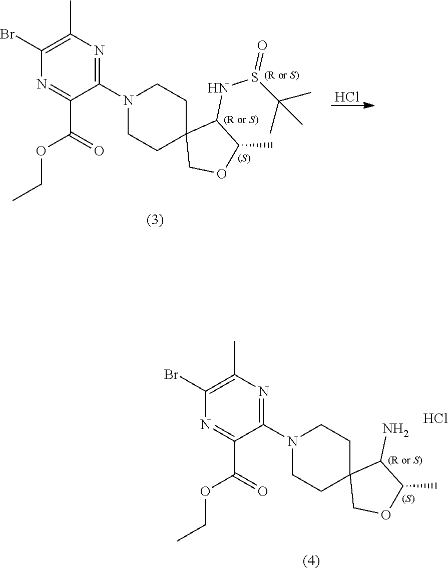

In a 3-neck round-bottom flask at room temperature was introduced ethyl 6-bromo-3-[(3S,4S)-4-[[(R)-tert-butylsulfinyl]amino]-3-methyl-2-oxa-8-azaspiro[4.5]decan-8-yl]-5-methyl-pyrazine-2-carboxylate (Compound (3a)) (92%, 430 mg, 0.76 mmol, 1 eq.) and EtOH (3.7 mL).

The reaction mixture was cooled down to 0-5° C., and then 0.87 mL HCl (1.25 M EtOH, 1.09 mmol, 1.5 eq.) was added. The reaction mixture was brought back to room temperature and stirred for 1 hr before being directly concentrated to dryness to afford the amine Compound (4) (S form) (381 mg, 94% yield (crude product)). LCMS analysis showed a peak at 412. ¹H NMR (600 MHz, DMSO-d₆) δ ppm 7.60-8.24 (m, 3H) 4.29-4.36 (m, 2H) 4.15-4.23 (m, 1H) 3.83-3.87 (m, 1H) 3.58-3.80 (m, 3H) 3.39-3.43 (m, 1H) 2.97-3.16 (m, 2H) 2.49 (s, 3H) 1.52-1.83 (m, 4H) 1.29-1.34 (m, 3H) 1.18-1.22 (m, 3H).

### Example 6. Preparation of Compound (5a)

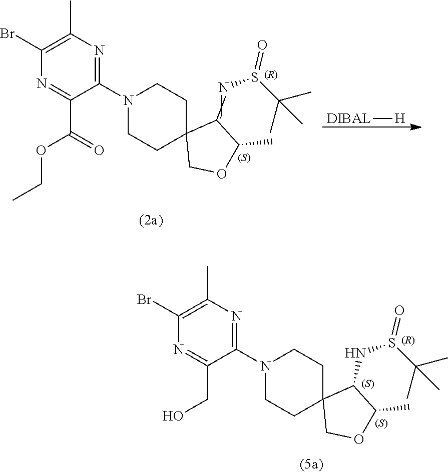

As discussed above in Example 2, the sulfinyl imine Compounds (2b) and (2a) were prepared from ethyl (S)-6-bromo-5-methyl-3-(3-methyl-4-oxo-2-oxa-8-azaspiro[4.5]decan-8-yl)pyrazine-2-carboxylate (Compound (1)) and (S)-sulfinamide (A1b) or (R)-sulfinamide (A1a), respectively. In each of the methods studied, the expected product (“EP” or “Compound (2a)” or “Compound (2b)”) was formed in addition to the corresponding isopropyl ester. In order to demonstrate that both esters (ethyl and isopropyl) could be reduced together in a mixture to the corresponding alcohol, a small amount of the mixed product of Compound (2a) was purified into the ethyl ester and isopropyl ester fractions by chromatography. Next, the reduction reaction was run on all three products: (1) the isolated isopropyl ester; (2) the isolated ethyl ester; and (3) the mixture of esters (ethyl and isopropyl). As shown below, the presence of the isopropyl ester did not affect the reduction to the corresponding alcohol.

Method 1—Reduction of isopropyl ester. In a 20 mL vial under N₂ atmosphere was introduced isopropyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (465 mg, 0.879 mmol) and anhydrous 2-methyltetrahydrofuran (5 mL). To the yellow solution, cooled down to −20° C., was added DIBAL-H (1.5 mL, 1 M THF, 1.5 mmol). The reaction mixture was stirred at −20° C. for 1.5 hr. LCMS monitoring showed full conversion of the sulfinyl-imine moiety. Three additional amounts of DIBAL-H (3×1.5 mmol) were introduced at −20° C. every 1 h 20 min to provide complete conversion of the isopropyl ester into the corresponding alcohol.

Once the conversion was complete, to the yellow reaction mixture was added Rochelle salt (10 mL, 20 vol.). The reaction mixture was allowed to reach room temperature. The thick suspension was stirred overnight at room temperature. The layers were separated, and the organic layer was washed twice with Rochelle salt solution (10 mL, 20 vol.). The clear yellow organic layer was dried and concentrated to dryness to afford crude Compound (5a) (478 mg, 97% yield). LCMS analysis confirmed the expected product.

Method 2—Reduction of Compound (2a)/isopropyl ester mixture. In a 20 mL vial under N₂ atmosphere was introduced an ethyl ester and isopropyl ester mixture (1:1) (280 mg) and anhydrous 2-methyltetrahydrofuran (2.8 mL). To the yellow solution, cooled down to −20° C., was added DIBAL-H (0.81 mL, 1 M THF, 0.815 mmol). The reaction mixture was stirred at −20° C. for 1 h. LCMS monitoring showed full reduction of the sulfinyl imine moiety. Two additional amounts of DIBAL-H (2×0.815 mmol) were introduced at −20° C. every 1 h to provide complete conversion of the esters (ethyl ester and isopropyl ester) into the corresponding alcohol (Compound (5a)).

Once the conversion was complete, to the yellow reaction mixture was added Rochelle salt (2.8 mL, 10 vol.) and the reaction mixture was allowed to reach room temperature. The suspension was stirred overnight at room temperature. The layers were separated, and the organic layer was washed twice with Rochelle salt solution (2.8 mL, 2×10 vol.). The clear yellow organic layer was dried and concentrated to dryness to afford crude Compound (5a) (263 mg, 92% yield). LCMS analysis confirmed the expected product.

Method 3—Reduction of ethyl ester (Compound (2a)). In a 100 mL 4-neck round-bottom flask under N₂ atmosphere was introduced ethyl 6-bromo-3-[(3S,4Z)-4-[(R)-tert-butylsulfinyl]imino-3-methyl-2-oxa-8-azaspiro[4.5]decan-8-yl]-5-methyl-pyrazine-2-carboxylate (95%, 1.99 g, 3.66 mmol) and anhydrous 2-methyltetrahydrofuran (20 mL). To the yellow solution, cooled down to −20° C., was added DIBAL-H (5.8 mL, 1 M THF, 5.8 mmol) over 7 min. The reaction mixture was stirred at −20° C. for 1 h. LCMS monitoring showed complete reduction of the sulfinyl imine moiety. Five additional amounts of DIBAL-H (2×1.5 eq. and 3×1 eq.) were introduced at −20° C. every 1 h to provide complete conversion of the ethyl ester intermediate (Compound (3a)) into the corresponding alcohol (Compound (5a)).

Once the conversion was complete, the reaction mixture was heated up to 0-5° C., and to the yellow reaction mixture was added Rochelle salt solution (20 mL). The reaction mixture was allowed to reach room temperature. The thick suspension was stirred for 45 min. at room temperature. The layers were separated, and the aqueous layer was extracted twice with MeTHF (10 mL). The combined organic layers were washed twice with Rochelle salt solution (20 mL). The clear yellow organic layer was dried over Na₂SO₄, filtered, and concentrated to dryness to afford the crude expected alcohol (Compound (5a)) (1.6 g, 67% yield, LCMS purity 73%). LCMS analysis showed a peak at expected mass of 474. ¹H NMR (500 MHz, DMSO-d₆, 300K) 6 ppm 5.45 (t, J=5.9 Hz, 1H), 5.09 (d, J=11.0 Hz, 1H), 4.42 (d, J=5.9 Hz, 2H), 4.12 (quin, J=6.4 Hz, 1H), 3.81 (d, J=8.6 Hz, 1H), 3.49 (d, J=8.8 Hz, 2H), 3.46-3.64 (m, 1H), 3.41 (dd, J=10.8, 6.1 Hz, 1H), 2.85-3.02 (m, 2H), 2.45 (s, 3H), 1.74-1.99 (m, 2H), 1.49-1.69 (m, 2H), 1.16 (s, 9H), 1.09 (d, J=6.6 Hz, 3H).

### Example 7. Preparation of Compound (6)

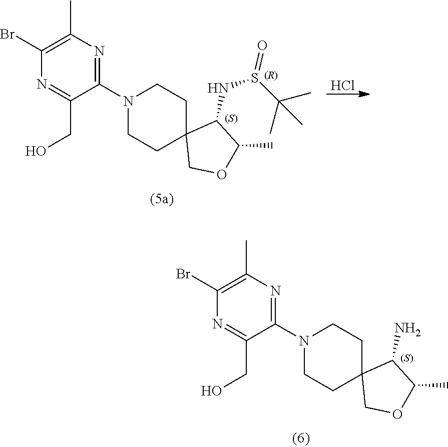

To a 1-neck round bottom flask was added Compound (5a) (113 mg, 0.23 mmol, 1 equiv.) and dry EtOH (1.5 mL). Next, HCl in EtOH (1.25 M, 0.27 mL, 0.34 mmol, 1.5 equiv.) was added at 0° C. The reaction mixture was placed under a nitrogen atmosphere and stirred at RT for 1 h., and then concentrated under vacuum to afford crude Compound (6) as an HCl salt (85.9 mg, LC-MS purity of 75%). ¹H NMR (600 MHz, DMSO-d) δ ppm 7.48-8.72 (m, 3H), 4.43 (s, 2H), 4.16-4.23 (m, 1H), 3.97-4.12 (m, 1H), 3.83-3.86 (m, 1H), 3.63-3.64 (m, 1H), 3.54-3.61 (m, 2H), 3.36-3.41 (m, 6H), 2.83-3.01 (m, 2H), 2.46 (s, 3H), 1.58-1.90 (m, 4H), 1.20-1.24 (m, 3H).

### Example 8. Preparation of Compound (6) (Scale-Up)

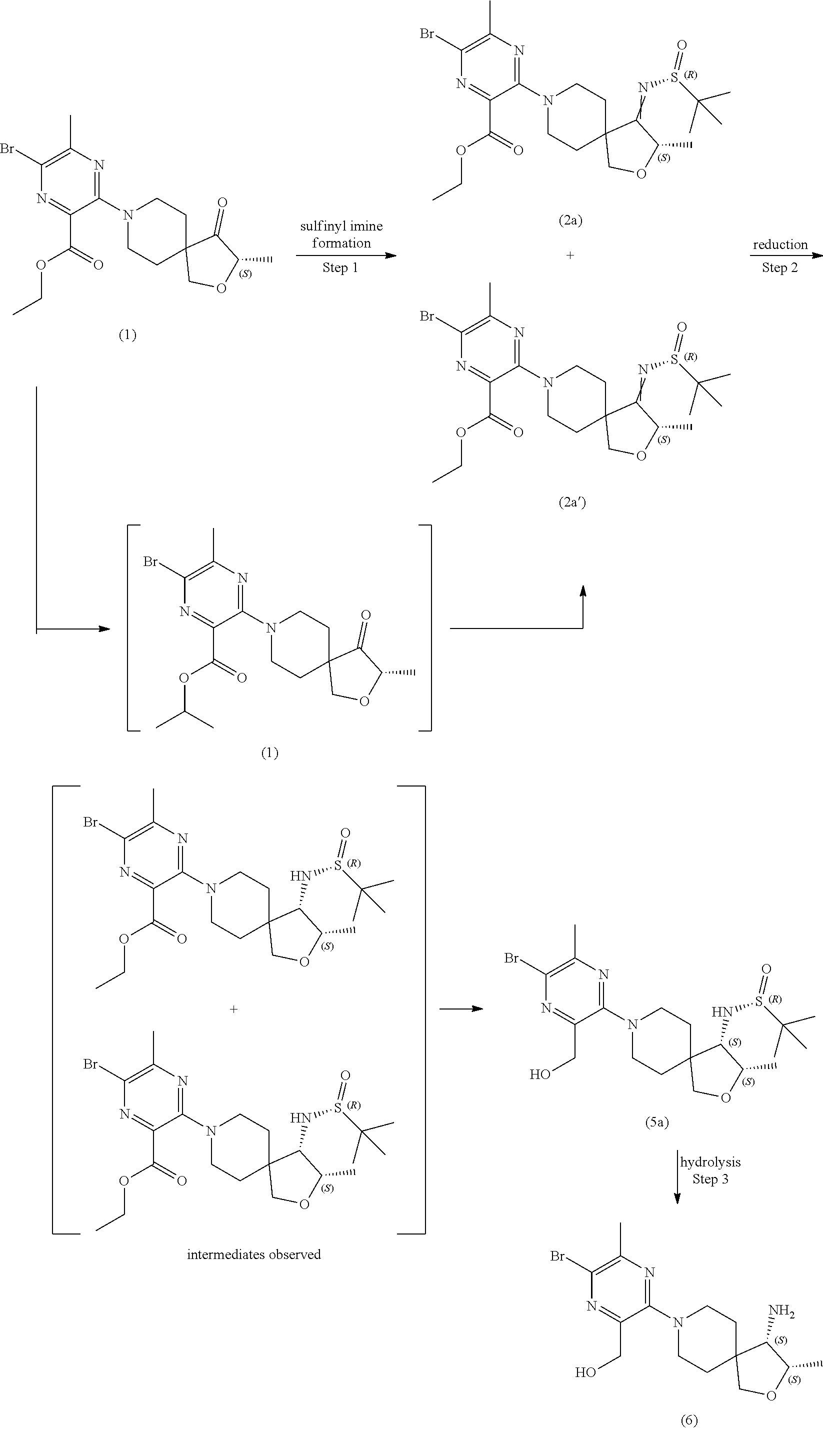

Step 1. Preparation of Compound (2a). To a 1 L, 4-neck, round-bottom flask equipped with thermometer, bleach trap, distillation system, and dropping funnel was added Compound (1) (25.00 g, purity 97%, 59.1 mmol) and (R)-2-methylpropane-2-sulfinamide (Compound (A1a) (9.49 g, 78.3 mmol, 1.3 eq.) in MeTHF (200 mL, 8 vol.) under Nitrogen and the resulting yellow cloudy solution was heated at 100° C. Tetraethoxytitanium (100%, 27.50 g, 0.121 mol, 2 eq.) in MeTHF (25 mL, 1 vol.) was added dropwise to the yellow solution via the dropping funnel, and the dropping funnel was rinsed with MeTHF (25 mL, 1 vol.). The reaction mixture was heated under reflux for 4-6 hr by distilling MeTHF to residual volume and refilling the reactor at 10 volumes with fresh MeTHF until conversion reached ˜96.4%-97%. Once the reaction was complete, the reaction mixture was cooled to 20-25° C. and water (17.5 mL, 8 eq. relative to Ti(OEt)₄) was added to the orange solution. The obtained suspension was filtered over a pad of Celite© and the cake was washed with MeTHF (4*5 vol). The yellow filtrate was kept as a MeTHF solution to afford Compound (2a) and the corresponding isopropyl ester (Compound (2a′)) in 93.4% yield as determined by HPLC analysis. LCMS analysis showed peaks at 514 (Compound (2a)) and 528 (Compound (2a′)). Certain parameters are summarized in Table 4.

TABLE 4

| Quantity of | HPLC | LCMS | | |
|---|---|---|---|---|
| MeTHE solution | Assay | Purity | KF | Chiral Purity |
| 567.4 g | 4.45% 2a | 77.57% 2a | 1.60% | 91.3% 2a |
| | 0.58% 2a′ | 7.07% 2a′ | | 7.7% 2a′ |
| | | | | 0.2% of 3S, S-sulfinimine*, |
| | | | | 0.8% of 3R, R-sulfinimine* |

*presence of diastereomer is related to the chiral purity of Compound (1) and Compound (A1a)

Compound (2a): ¹H NMR (DMSO-d₆, 500 MHz): δ (ppm) 4.88 (q, J=6.4 Hz, 1H), 4.31 (q, J=7.1 Hz, 2H), 3.98 (q, J=9.5 Hz, 2H), 3.68-3.90 (m, 2H), 3.02-3.21 (m, 2H), 2.48 (s, 3H), 1.56-1.87 (m, 4H), 1.42 (d, J=6.8 Hz, 3H), 1.27-1.33 (m, 3H), 1.17 (s, 9H).

Compound (2a′): ¹H NMR (500 MHz, DMSO-d₆) δ 5.18-5.11 (m, 1H), 5.11-5.03 (m, 1H), 4.05-3.87 (m, 2H), 3.85-3.70 (m, 2H), 3.23-3.11 (m, 2H), 2.49 (br s, 3H), 1.95-1.58 (m, 4H), 1.40-1.36 (m, 3H), 1.36-1.31 (m, 6H), 1.21 (s, 9H).

Mixture of Compound (2a) +˜10% Compound (2a′): ¹H NMR (DMSO-d₆, 500 MHz): δ (ppm) 5.05-5.14 (m, 1H), 4.31 (q, J=7.3 Hz, 2H), 3.87-4.00 (m, 2H), 3.71-3.83 (m, 2H), 3.07-3.19 (m, 2H), 2.48 (s, 3H), 1.72-1.82 (m, 2H), 1.60-1.70 (m, 2H), 1.35 (d, J=6.8 Hz, 3H), 1.28-1.32 (m, 3H), 1.18 (s, 9H).

Step 2. Preparation of Compound (5a). To a 1 L, 4-neck, round-bottom flask equipped with thermometer, bleach trap, distillation system, and peristaltic pump was added a MeTHF solution of 567.4 g containing Compound (2a) (4.5% w/w, 49.0 mmol) and Compound (2a′) (0.58% w/w, 6.18 mmol) under N₂. The solution was azeotroped twice and concentrated at 100° C. to reach an equivalent of 10% w/w MeTHF solution a water content around 0.30%. The reaction mixture was then cooled down to −20° C. and 1 M diisobutylaluminum hydride (331 mL, 0.331 mol, 6 eq., calculated relative to both Compounds (2a) and (2a′)) was added dropwise to the yellow solution. Upon completion of the reaction (˜2 h after the end of addition), the reaction mixture was quenched at −20° C. with the controlled addition of 331 mL of MeOH; then 13.8 mL of water; 13.8 mL of sodium hydroxide (15% w/w); and 33.1 mL of water. The reaction mixture was warmed to 20-25° C. and stirred over 3-4 h. The suspension obtained was filtered and the cake was washed with MeTHF (3*5 vol). The resulting yellow filtrate was kept as a MeTHF solution. Compound (5a) was obtained in 80.5% yield, as determined by HPLC assay analysis of MeTHF solution. LCMS analysis showed a peak at 474 (Compound (5a)). Certain parameters are summarized in Table 5.

TABLE 5

| | HPLC | LCMS | |
|---|---|---|---|
| Quantity | Assay | Purity | Chiral Purity |
| 773.4 g | 2.73% 5a | 67.75% 5a | 97.3% 5a (3S,4S and R- |
| | | | terbutanesulfinamide) |
| | | | 0.8% of 3S,4R, S- |
| | | | terbutanesulfinamide, |
| | | | 1.2% of 3S,4S, S- |
| | | | terbutanesulfinamide, |
| | | | 0.4% of 3R,4R, R- |
| | | | terbutanesulfinamide |

Compound (5a): ¹H NMR (500 MHz, DMSO-d₆, 300 K) δ ppm 5.45 (t, J=5.9 Hz, 1H), 5.09 (d, J=11.0 Hz, 1H), 4.42 (d, J=5.9 Hz, 2H), 4.12 (quin, J=6.4 Hz, 1H), 3.81 (d, J=8.6 Hz, 1H), 3.49 (d, J=8.8 Hz, 2H), 3.46-3.64 (m, 1H), 3.41 (dd, J=10.8, 6.1 Hz, 1H), 2.85-3.02 (m, 2H), 2.45 (s, 3H), 1.74-1.99 (m, 2H), 1.49-1.69 (m, 2H), 1.16 (s, 9H), 1.09 (d, J=6.6 Hz, 3H).

Step 3. Preparation of Compound (6). To a 500 mL, 4-neck, round-bottom flask equipped with thermometer, bleach trap, distillation system, and dropping funnel was added a 773.4 g of a MeTHF solution containing 2.7% w/w of Compound (5a) (44.4 mmol) under N₂. The solution was distilled at 100° C. to obtain a residual 210 mL of MeTHF solution. Water (53 mL, 2.5 vol) was added and the reaction mixture was cooled down to 0-5° C. Aqueous concentrated hydrogen chloride (36%, 40 mL, 0.444 mol, 10 eq.) was added dropwise. At the end of the addition, the temperature was increased to 20-25° C. After 2 h of stirring, the reaction was complete.

The acidic aqueous layer was separated from the MeTHF layer, cooled down to 0-5° C. and residual MeTHF was removed under a strong bubbling of N₂. Then sodium hydroxide (30%) was added to reach a pH of 12. The reaction mixture was warmed to 20-25° C. and precipitation occurs. The solid was filtered and rinsed with water (3*5 vol) to afford Compound (6) with 82.4% yield. LCMS analysis showed a peak at 370 (Compound (6)). Certain parameters are summarized in Table 6.

TABLE 6

| | HPLC | LCMS | |
|---|---|---|---|
| Quantity | Assay | Purity | Chiral Purity |
| 15.51 g | 87.68% 6 | 89.82% 6 | 98.4% of 6 |
| | | | 0.6% (3S,4R dia) |
| | | | 1.0% (3R,4S dia) |

Compound (6): ¹H NMR (DMSO-d₆, 600 MHz) δ 5.42 (br t, 1H, J=5.7 Hz), 4.42 (d, 2H, J=5.3 Hz), 3.9-4.1 (m, 1H), 3.63 (d, 1H, J=8.5 Hz), 3.46 (d, 3H, J=8.4 Hz), 3.0-3.2 (m, 2H), 2.87 (d, 1H, J=5.1 Hz), 2.44 (s, 3H), 1.80 (ddd, 1H, J=3.5, 9.6, 13.3 Hz), 1.69 (ddd, 1H, J=3.7, 9.5, 13.3 Hz), 1.5-1.6 (m, 2H), 1.29 (br d, 2H, J=1.2 Hz), 1.07 (d, 3H, J=6.5 Hz).

### Example 9. Preparation of Compound (7)

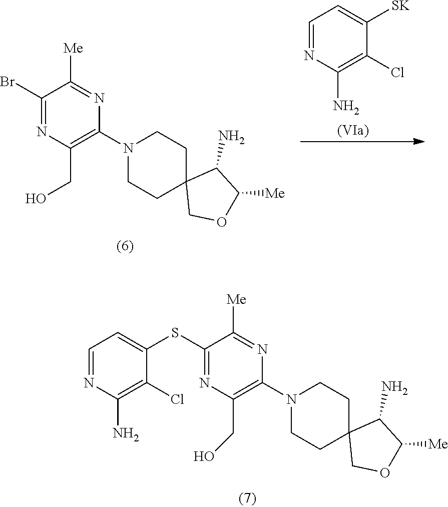

Compound (7), or a salt thereof, was prepared by coupling Compound (6) with Compound (VIa).

A 5 L flask was charged with Compound (6) (100 g, 1.0 equiv.), Compound (VIa) (64.22 g, 1.2 equiv.), CuI (10 g, 0.2 equiv.), and pyridine (980 g). The mixture was de-gassed with N₂ and heated to 110-120° C. for 20-30 h. The reaction mixture was cooled to 20-30° C. and filtered through diatomite. The cake was washed with pyridine (400 g) and the filtrate was reduced under vacuum. Next, MeOH (237 g) and DCM (3990 g) were added to the filtrate and stirred. To the resulting solution was added NH₃·H₂O (10 wt. %, 1000 g) and the reaction mixture was stirred at 20-30° C. for 1-2 h. The aqueous phase was washed with DCM. To the combined organic phases was added MeOH (158 g) and the organic phase was filtered via 0.45 μm filter. The 0.45 μm filter was washed with DCM/MeOH (4:1 v/v) and the organic phase was concentrated under vacuum. DCM/MeOH (4:1 v/v) was added, followed by dropwise addition of MTBE (111 g) over 1 h. Additional MTBE (703 g) was added over about 6 h, and the reaction mixture was stirred for 8-24 h at 20-30° C. The mixture was filtered and the cake was dried to afford Compound (7) in 55-85% yield.

### Example 10. Preparation of 3-Chloro-2-(Methylamine)Pyridine-4-Thiol Potassium Salt (Compound (VIa))

Route A. Compound (VIa) was prepared according to the reactions shown below:

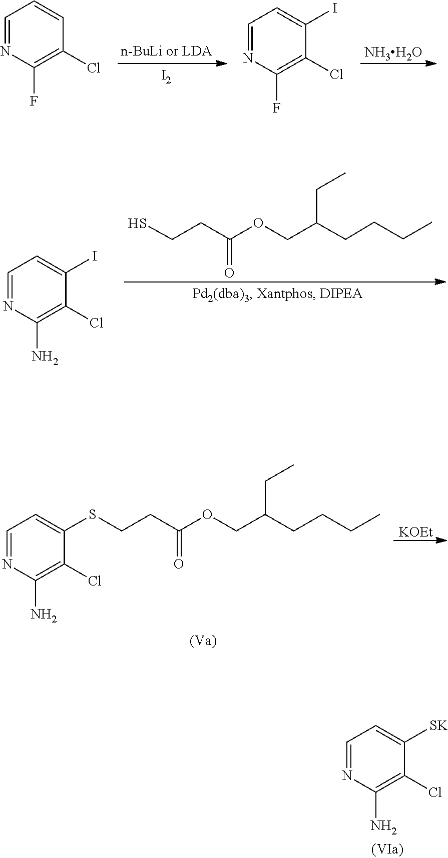

Step 1.

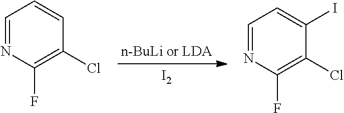 [SMILES: Fc1ncccc1Cl ; "I2" ; Fc1nccc(I)c1Cl]

To a 500 L reactor was charged 2-MeTHF (50 mL) and degassed with nitrogen. The mixture was charged LDA (2.0 M, 102.6 L, 1.2 equiv), then cooled to −70 to −60° C. To the mixture was added a solution of 3-Chloro-2-fluoropyridine (22.5 kg, 171 mol) in 2-MeTHF (40 kg) at −70 to −60° C. The mixture was stirred at −70 to −60° C. for 30 minutes. To the mixture was added a solution of 12 (47.7 kg, 1.1 equiv) in 2-MeTHF (80 mL) at −70 to −60° C. over 30 minutes. The mixture was stirred at −70 to −60° C. for 60 minutes. TLC (PE:EA=10:1, Rf=0.5) showed full conversion. The reaction mixture was added to another reactor contained 225 kg 1 N HCl solution at 0-20° C. The phases were separated, and the aqueous phase was extracted with EA (79 kg). The combined organic phase was washed with 112.5 kg 30% Na₂S₂O₃ solution. The organic phase was dried over anhydrous MgSO₄ (45 kg), filtered. The cake was rinsed with EA (11.25 kg). The combined filtrate was concentrated under reduced to pressure to about 2 vol. To the mixture was added 45 kg MTBE then distilled under reduced pressure at 45±5° C. to about 2 vol, which was repeated for 3 times. The mixture was cooled to 20±5° C. and filtered after 1 hour. The cake was rinsed with 5.6 kg MTBE. The solid was dried under reduced pressure at 45±5° C. ° C. to give 3-chloro-2-fluoro-4-iodopyridine (32.0 kg, 99.6% purity, 72.7% yield).

Step 2.

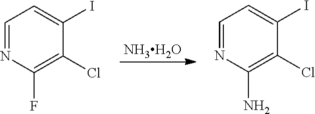

To a 100 L pressure reaction was added 3-chloro-2-fluoro-4-iodopyridine (11.0 kg, 42.7 mol) and ammonium hydroxide solution (33 kg). DMSO (36.3 kg) was slowly added to the mixture. The mixture was warmed to 80±5° C. and stirred for 5 hours. TLC (PE:EA=2:1, Rf=0.3) showed full conversion. After being cooled to 25±5° C., the mixture was added to water (110 kg) in a 500 L reactor, and stirred at 25±5° C. for 1 hour. The mixture was filtered. The cake was reslurried two times water (55 kg×2) at 20±5° C. The cake was dried under reduced pressure at 45±5° C. ° C. to give 3-Chloro-4-iodo-2-pyridinamine (30.8 kg, 99.3% purity, 94.4% yield) as a solid.

Step 3. Synthesis of (Va).

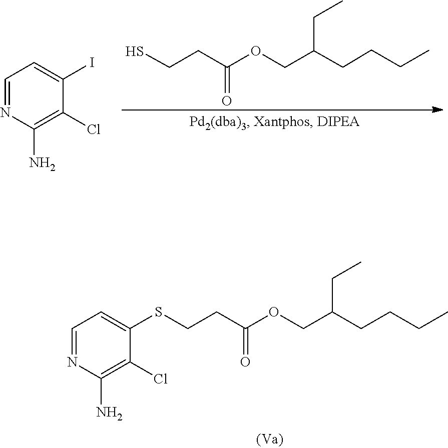 [SMILES: Nc1nccc(I)c1Cl ; CCCCC(CC)COC(=O)CCS ; CCCCC(CC)COC(=O)CCSc1ccnc(N)c1Cl]

To a 1000 L reactor was added dioxane (121 kg), 3-chloro-4-iodo-2-pyridinamine (23.4 kg, 92.0 mol), 2-Ethylhexyl 3-mercaptopropionate (21.1 kg, 96.6 mol, 1.05 equiv), DIPEA (23.8 kg, 2 equiv), Xantphos (0.267 kg, 0.005 equiv), and Pd(OAc)₂ (0.103 kg, 0.005 equiv). After being purged with nitrogen for three times, the mixture was warmed to 95±5° C. and stirred for 5 hours. TLC (PE:EA=2:1, Rf=0.3) showed full conversion. After cooled to 20±5° C., the mixture was added 2-MeTHF (187 kg) and water (117 kg), and stirred for 30 minutes. After phase separation, the organic phase was washed with water (117 kg). The combined aqueous phase was extracted with 2-MeTHF (47 kg). The combined organic phase was dried over anhydrous MgSO₄ (37 kg) and filtered through a pad of silica gel (100-200 mesh, 9.5 kg). The silica gel pad was rinsed with MeTHF (70 kg×2). The combined filtrate was distilled under reduced pressure to about 2 vol. The mixture was added EA (47 kg) and distilled under reduced pressure at 45±5° C. to about 2 vol. The mixture was added EA (47 kg) and distilled under reduced pressure at 45±5° C. to about 2 vol. The mixture was added EA (84 kg) and distilled under reduced pressure at 45±5° C. to about 2.5 vol. The mixture was cooled to 15±5° C. After stirred for 1 hour, the mixture was filtered and rinsed with 9 kg EA. The cake was dried at 45±5° C. to give 2-ethylhexyl 3-((2-amino-3-chloropyridin-4-yl)thio)propanoate (Va) (28.6 kg, 97.9% purity, 90.2% yield).

Step 4.

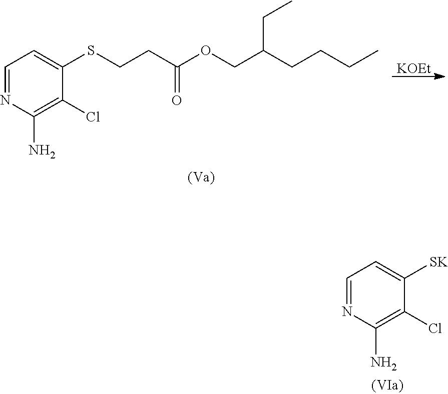

To a reactor was charged 2-ethylhexyl 3-((2-amino-3-chloropyridin-4-yl)thio)propanoate (Va) (38.1 kg, 1 equiv) and 2-MeTHF (312 kg). The resulting mixture was maintained at 15-25° C. for 2 hour at which point a clear solution formed. To this was charged KOEt (˜24% w/w in EtOH) (41.8 kg, 1.1 equiv) over two hours, maintaining a temperature of 15-25° C., followed by additional 2-MeTHF (4 kg). The reaction was maintained at 15-25° C. for 4 hours at which point UPLC monitoring showed reaction completion.

To the reactor was charged MTBE (147 kg). The resulting suspension was maintained at 15-25° C. for 4 hours and then filtered, washing the cake with more MTBE (84 kg). The cake was dried for 24 hours at ≤30° C. to afford potassium 2-amino-3-chloropyridine-4-thiolate (VIa) (21.65 kg, 96.9% a/a UPLC purity, 95.8% w/w assay, 95% assay-corrected yield).

Route B. Compound (VIa) was prepared according to the scheme shown below:

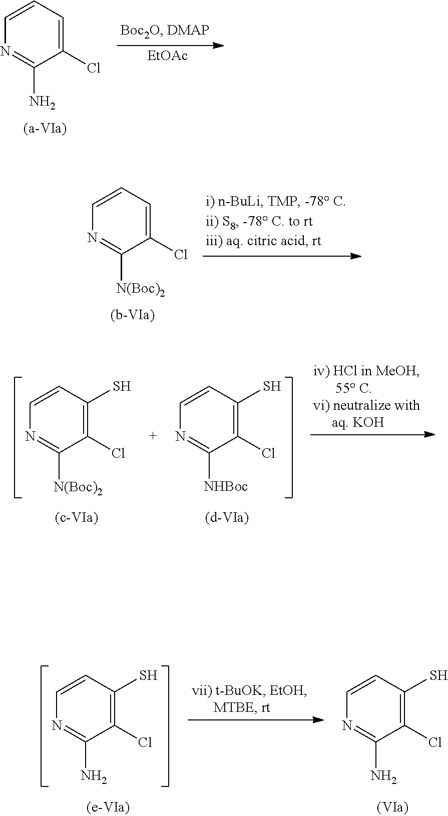

Step 1. Synthesis of (b-VIa).

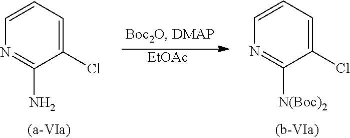

To a flask was charged 3-chloropyridin-2-amine (a-VIa) (100 g, 1.0 equiv), DMAP (5.6 g, 0.1 equiv), and EtOAc (700 mL, 7.0 vol). The resulting mixture was maintained at 25-30° C. for 10 minutes. To this was charged Boc₂O (424 g, 2.5 equiv) over −1 hour, maintaining a temperature of 25-30° C., during which time a lot of gas was released. The resulting mixture was maintained at 25-30° C. for 7 hours at which point HPLC monitoring showed reaction completion.

To the reaction was charged 10% w/w aq. citric acid (200 mL, 2.0 vol) and the resulting mixture was maintained at 25-30° C. for 10 minutes. The layers were separated and the aqueous layer was extracted with EtOAc (300 mL, 3.0 vol). The combined organic layers were washed with 10% w/w aq. NaCl (200 mL, 2.0 vol) and then concentrated to ˜2 vol under reduced pressure at 45° C. To the concentrate was added n-heptane (300 mL, 3.0 vol) during which time a lot of solid precipitated. The suspension was concentrated to ˜2 vol under reduced pressure at 45° C. To the concentrate was added n-heptane (200 mL, 2.0 vol) and the resulting mixture was maintained at 25-30° C. for 30 minutes and then filtered, washing the cake with n-heptane (100 mL, 1.0 vol). The cake was dried under reduced pressure at 45° C. for 4 h to afford tert-butyl (tert-butoxycarbonyl)(3-chloropyridin-2-yl)carbamate (b-VIa) (225 g, 98.3% a/a HPLC purity, 86% corrected yield).

¹H NMR (400 MHz, CDCl₃) δ 8.43 (dd, J=4, 8 Hz, 1H), 7.80 (dd, J=4, 8 Hz, 1H), 7.26 (dd, J=4, 8 Hz, 1H), 1.41 (s, 18H).

MS (ESI+): calculated, 329.13; found, 329.1.

Step 2. Synthesis of (VIa).

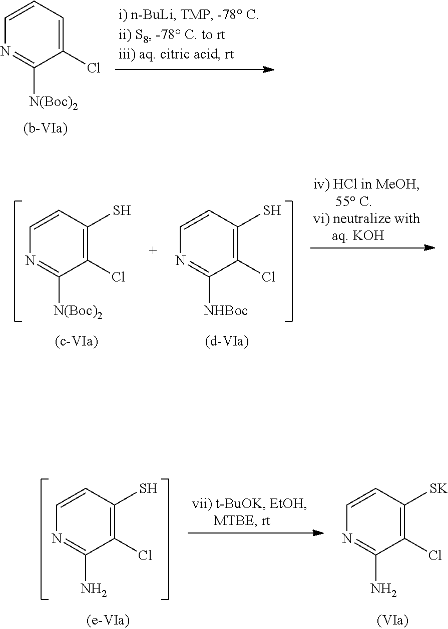

To a flask was charged 2,2,6,6-tetramethylpiperidine (TMP) (103 g, 1.5 equiv) and THE (1.6 L, 10 vol). The flask was evacuated and backfilled with nitrogen 3 times. The solution was cooled to −80 to −70° C. To this was charged n-BuLi (311 mL, 1.5 equiv, 2.5 mol/L), maintaining a temperature of ˜80 to −70° C. The reaction was maintained at −80 to −70° C. for 30 minutes. To this was charged a solution of tert-butyl (tert-butoxycarbonyl)(3-chloropyridin-2-yl)carbamate (b-VIa) (160 g, 1.0 equiv) in THE (800 mL, 5 vol) over 60 minutes, maintaining a temperature of −80 to −70° C. The resulting mixture was maintained at −80 to −70° C. for 2 hours. To this was charged Ss (23.4 g, 1.5 equiv), maintaining a temperature of −80 to −70° C. The resulting mixture was warmed to 20-30° C. and maintained at 20-25° C. for 2 hours at which point HPLC monitoring showed reaction completion.

The reaction was quenched with water (800 mL, 5 vol), maintaining a temperature of 20-30° C. The mixture was extracted with MTBE (1.6 L, 10 vol) and the organic layer was washed with water (800 mL, 5 vol). To the combined aqueous layers was added 25% w/w aq. Na₂SO₃ (1.6 L, 10 vol), maintaining a temperature of 20-30° C. The solution was maintained at 20-30° C. for 3 hours. The pH was then adjusted to 5-6 with 30% w/w aq. citric acid (1.2 L, 7.5 vol). This was then extracted with 2-MeTHF (800 mL×3). The combined organic layers were washed with 20% w/w aq. NaCl (800 mL, 5 vol).

To the reaction mixture was charged 36% w/w HCl in MeOH (395 g, 8.0 equiv). The resulting mixture was maintained at 50-60° C. for 4 hours during which time white solid precipitated and HPLC monitoring showed reaction completion.

The MeOH and 2-MeTHF were removed by distillation under reduced pressure at ca. 45° C. To the resulting residue was added water (320 mL, 2.0 vol). This was concentrated to ˜2 vol under reduced pressure at 50-55° C. The pH was adjusted to 7 with 40% w/w aq. KOH (ca. 280 mL). The resulting suspension was filtered, washing the cake with water (160 mL). The cake was dried under reduced pressure at 55° C. for 4 hours to afford 2-amino-3-chloropyridine-4-thiol (e-VIa) (64.6 g, 94.6% HPLC purity) that was used directly in next step.

The crude 2-amino-3-chloropyridine-4-thiol (e-VIa) was suspended in 2-MeTHF (260 mL, 4 vol). To the suspension was added t-BuOK (47.6 g, 1.05 equiv) in EtOH (142 g), maintaining a temperature of 20-30° C., during which time a clear solution formed. This was maintained at 20-30° C. for 30 minutes. To this was charged MTBE (260 mL, 4.0 vol) and the resulting mixture was maintained for 30 minutes until precipitation had stopped. To this was then charged additional MTBE (710 mL, 11 vol) and the resulting suspension was maintained for 60 minutes. The suspension was then filtered, washing the cake with MTBE (320 mL). The cake was dried under reduced pressure at 45° C. for 4 hours to afford potassium 2-amino-3-chloropyridine-4-thiolate (VIa) (72 g, 98.8% a/a HPLC purity, 74% yield).

¹H NMR for potassium 2-amino-3-chloropyridine-4-thiolate (VIa) (400 MHz, DMSO-d₆) δ 7.04 (d, J=8 Hz, 1H), 6.47 (d, J=8 Hz, 1H), 5.04 (br s, 2H).

¹³C NMR for potassium 2-amino-3-chloropyridine-4-thiolate (VIa) (100 MHz, DMSO-d₆) δ 169.3, 154.6, 140.8, 122.3, 116.5.

Potassium (K) content for potassium 2-amino-3-chloropyridine-4-thiolate (VIa) (ICP-MS): calculated, 196.76 g/kg; found, 194.33 g/kg.

MS for potassium 2-amino-3-chloropyridine-4-thiolate (VIa) (ESI+): calculated, 160.99; found, 161.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. The disclosures of all patent and scientific literature cited herein are expressly incorporated herein in their entirety by reference. To the extent that any incorporated material is inconsistent with the express content of this disclosure, the express content controls.

1. A method of preparing a compound of Formula (6): 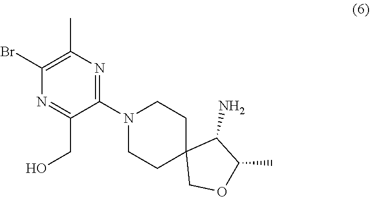 [SMILES: Cc1nc(N2CCC3(CC2)CO[C@@H](C)[C@H]3N)c(CO)nc1Br]
or a salt thereof, comprising:
reacting a compound of Formula (5): 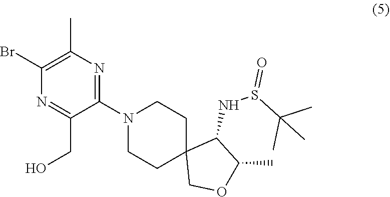 [SMILES: Cc1nc(N2CCC3(CC2)CO[C@@H](C)[C@H]3NS(=O)C(C)(C)C)c(CO)nc1Br]
with an acid to form the compound of Formula (6) or a salt thereof.

2. (canceled)

3. The method of claim 1, wherein the compound of Formula (5) is the compound of Formula (5a):

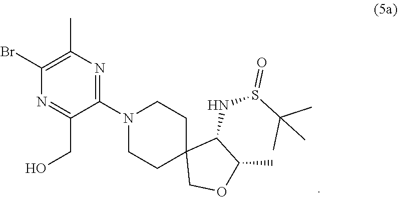 [SMILES: Cc1nc(N2CCC3(CC2)CO[C@@H](C)[C@H]3N[S@](=O)C(C)(C)C)c(CO)nc1Br]

4. The method of claim 1 or 3, wherein the compound of Formula (5) is prepared by reacting a compound of Formula (2): 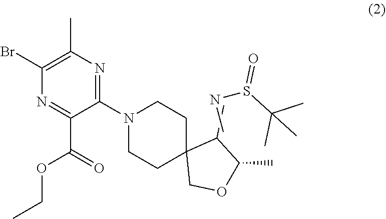
with a reducing agent to form the compound of Formula (5).

5. (canceled)

6. (canceled)

7. The method of claim 4, wherein the compound of Formula (2) is the compound of Formula (2a): 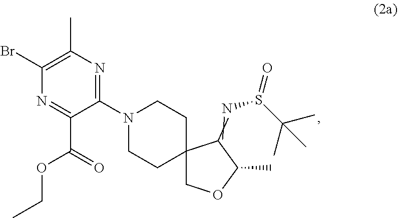 [SMILES: CCOC(=O)c1nc(Br)c(C)nc1N1CCC2(CC1)CO[C@@H](C)C2=N[S@](=O)C(C)(C)C]
and the compound of Formula (5) is the compound of Formula (5a):

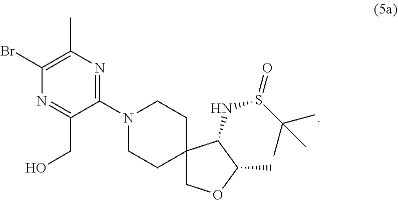 [SMILES: Cc1nc(N2CCC3(CC2)CO[C@@H](C)[C@H]3N[S@](=O)C(C)(C)C)c(CO)nc1Br]

8. The method of claim 4 or 7, wherein the compound of Formula (2) is prepared by reacting the compound of Formula (1): 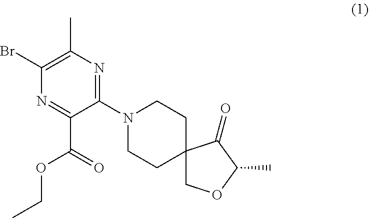 [SMILES: CCOC(=O)c1nc(Br)c(C)nc1N1CCC2(CC1)CO[C@@H](C)C2=O]
with the compound of Formula (A1): 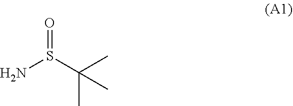
and a titanium alkoxide reagent to form the compound of Formula (2).

9. (canceled)

10. The method of claim 8, wherein the compound of Formula (1) is prepared by reacting a compound of Formula (II): 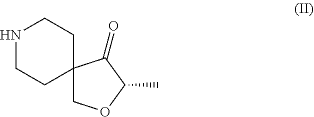
or a salt thereof,
with the compound of Formula (IV): 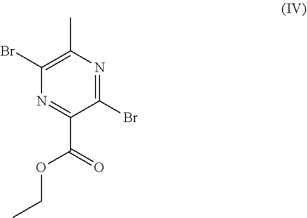
to form the compound of Formula (1).

11. The method of claim 10, wherein the reaction of the compound of Formula (II), or a salt thereof, with the compound of Formula (IV) further comprises a base.

12. (canceled)

13. The method of claim 10 or 11, wherein the compound of Formula (II), or a salt thereof, is prepared by reacting a compound of Formula (I): 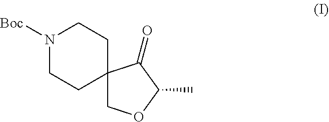 [SMILES: C[C@@H]1OCC2(CCN(C(=O)OC(C)(C)C)CC2)C1=O]
with an acid to form the compound of Formula (II) or a salt thereof.

14. (canceled)

15. The method of any one of claims 10, 11, and 13, wherein the compound of Formula (IV) is prepared by reacting a compound of Formula (III): 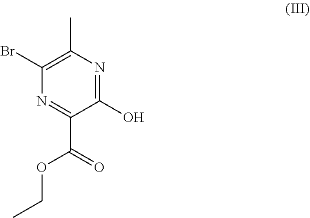
with POBr₃ to form the compound of Formula (IV).

16. A method of preparing a compound of Formula (7): 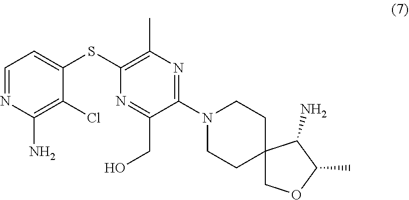 [SMILES: Cc1nc(N2CCC3(CC2)CO[C@@H](C)[C@H]3N)c(CO)nc1Sc1ccnc(N)c1Cl]
or a salt thereof, comprising reacting the compound of Formula (6), or a salt thereof, prepared according to any one of claims 1-15 with a compound of Formula (VI): 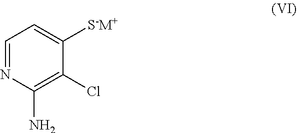
wherein M⁺ is Li⁺, Na⁺, or K⁺,
to form the compound of Formula (7) or a salt thereof.

17. The method of claim 16, wherein the reaction of the compound of Formula (6), or a salt thereof, with the compound of Formula (VI) further comprises a copper salt.

18. The method of claim 16 or 17, wherein the compound of Formula (VI) is prepared by reacting a compound of Formula (V): 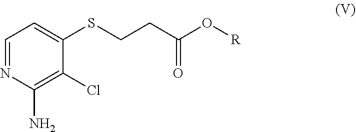
wherein R is C₁-C₁₂ alkyl,
with a base to form the compound of Formula (VI).

19. The method of claim 18, wherein the compound of Formula (V) is the compound of Formula (Va):

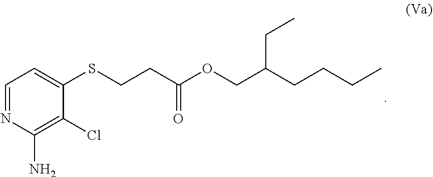 [SMILES: CCCCC(CC)COC(=O)CCSc1ccnc(N)c1Cl]

20. (canceled)

21. A method of preparing a compound of Formula (7), or a salt thereof, comprising the following steps:

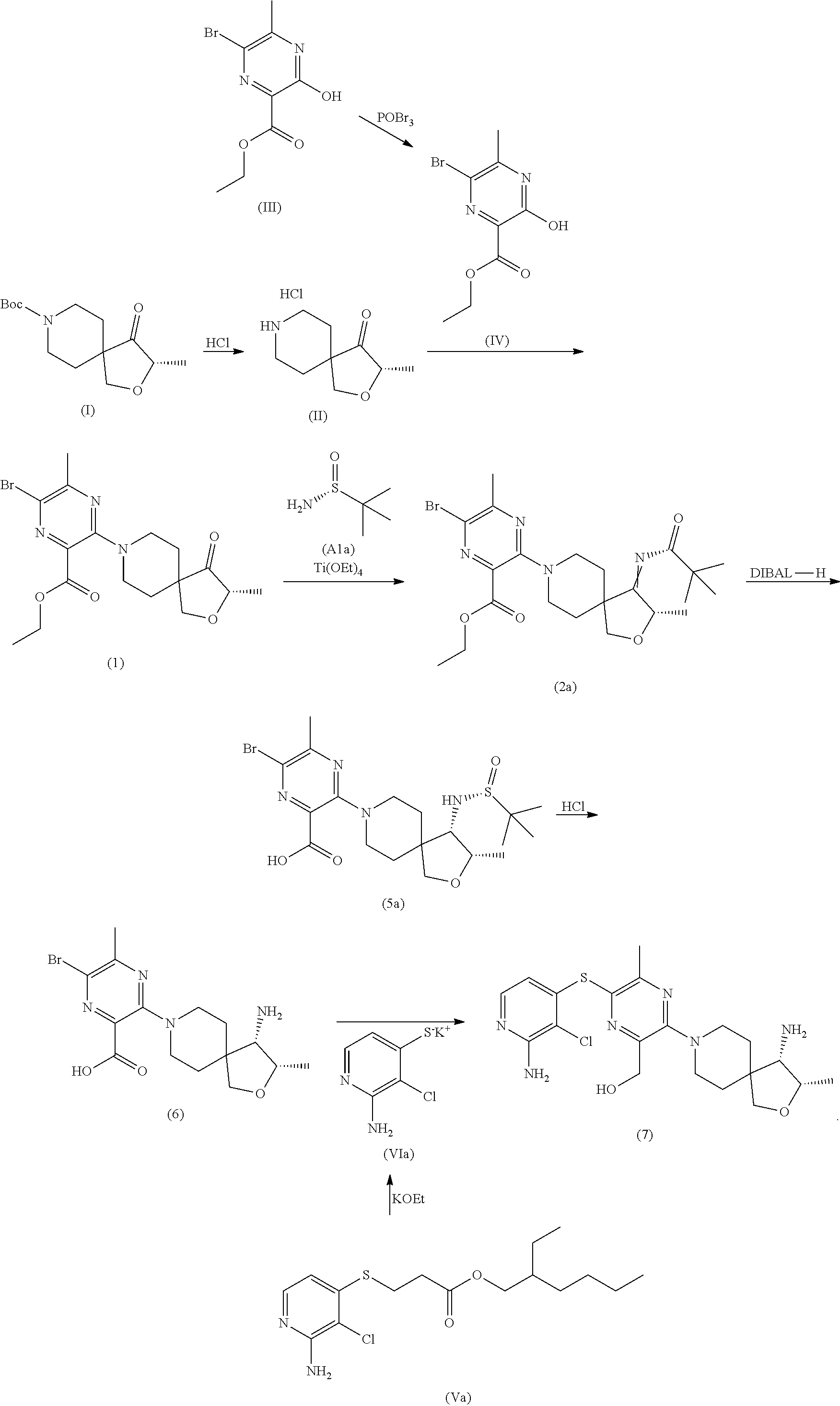

22. A method of preparing a compound of Formula (4): 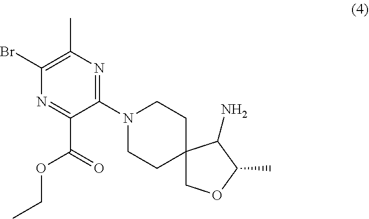 [SMILES: CCOC(=O)c1nc(Br)c(C)nc1N1CCC2(CC1)CO[C@@H](C)C2N]
or a salt thereof, comprising:
reacting a compound of Formula (3): 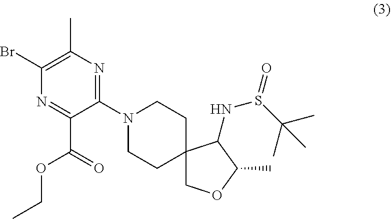 [SMILES: CCOC(=O)c1nc(Br)c(C)nc1N1CCC2(CC1)CO[C@@H](C)C2NS(=O)C(C)(C)C]
with an acid to form the compound of Formula (4) or a salt thereof.

23. The method of claim 22, wherein the compound of Formula (3) is prepared by reacting a compound of Formula (2): 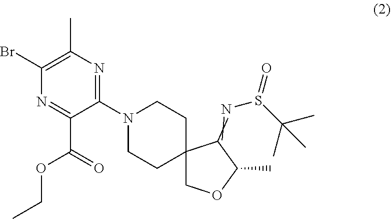 [SMILES: CCOC(=O)c1nc(Br)c(C)nc1N1CCC2(CC1)CO[C@@H](C)C2=NS(=O)C(C)(C)C]
with a reducing agent to form the compound of Formula (3).

24. A compound selected from the group consisting of Formula (1), Formula (2), and Formula (3):

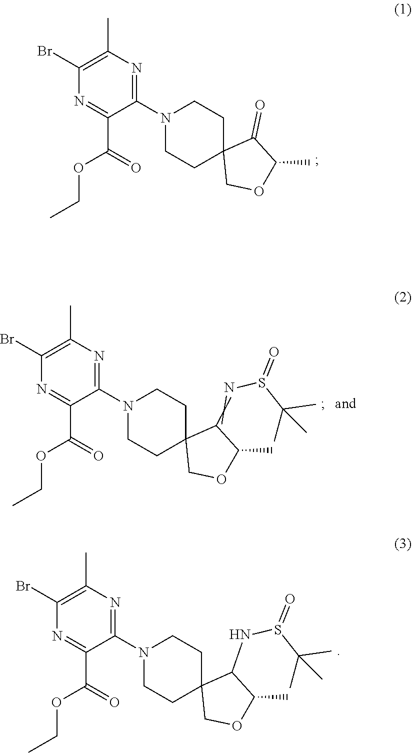 [SMILES: CCOC(=O)c1nc(Br)c(C)nc1N1CCC2(CC1)CO[C@@H](C)C2=O ; CCOC(=O)c1nc(Br)c(C)nc1N1CCC2(CC1)CO[C@@H](C)C2=NS(=O)C(C)(C)C ; CCOC(=O)c1nc(Br)c(C)nc1N1CCC2(CC1)CO[C@@H](C)C2NS(=O)C(C)(C)C]

25. (canceled)

26. (canceled)
